(19) United States
(12) Patent Application Publication
(10) Pub. No.: US 2023/0090094 A1
(43) Pub. Date: Mar. 23, 2023

(54) HEMP EXTRACT FOR TREATMENT OF PAIN, CANCER AND EPILEPSY IN ANIMALS

(71) Applicant: ElleVet Sciences, South Portland, ME (US)

(72) Inventors: Christian KJAER, South Portland, ME (US); Amanda HOWLAND, South Portland, ME (US); Joseph WAKSHLAG, South Portland, ME (US)

(21) Appl. No.: 17/597,259

(22) PCT Filed: Jul. 2, 2020
(86) PCT No.: PCT/US20/40614

§ 371 Date: Dec. 30, 2021

Related U.S. Application Data
(60) Provisional application No. 62/870,043, filed on Jul. 2, 2019.
(60) Provisional application No. 62/962,114, filed on Jan. 16, 2020.

(51) Int. Cl. A61K 31/352; A61K 45/06; A61K 47/44; A61K 9/00; A61K 31/015; A61K 31/336; A61K 31/685; A61K 31/055; A61P 29/00; A61K 36/73

(52) U.S. Cl. CPC A61K 31/352; A61K 45/06; A61K 47/44; A61K 9/0056; A61K 31/015; A61K 31/336; A61K 31/685; A61K 31/055; A61P 29/00; A61K 36/73

(57) ABSTRACT

The present disclosure relates to methods of treating epilepsy, cancer, and post-operative pain in veterinary subjects using pharmaceutical compositions and dosage forms comprising hemp extract.

### CROSS-REFERENCE TO RELATED APPLICATIONS

This application is a § 371 National Stage filing of PCT International Application No. PCT/US2020/040614, filed Jul. 2, 2020 which claims the benefit of U.S. Provisional Patent Application Ser. Nos. 62/870,043, filed Jul. 2, 2019, and 62/962,114, filed Jan. 16, 2020, the entire disclosure of each of which is hereby incorporated herein by reference.

### BACKGROUND

A recent survey by the American Holistic Veterinary Medical Association revealed that almost 60% of people who buy hemp products online use these products for their dogs. Industrial hemp products that are low in THC (0.3%) and higher in other cannabinoids are reported to have health benefits including analgesic, anti-inflammatory, anti-anxiolytic, and anti-epileptic; and are legal according to the industrial hemp act. There are numerous on-line companies selling hemp products including CBD oil claiming they are safe and effective for various medical conditions in both pets and people. There is very little published data to support these claims and no data to indicate the safety of using CBD oil concurrently with chemotherapy in veterinary patients. In the absence of an optimal treatment for these dogs for pain, epilepsy, or cancer, other potentially efficacious pharmacological agents, including cannabinoids, are often sought.

### SUMMARY

The present disclosure is directed toward compositions comprising cannabidiol and their use for the treatment of pain, epilepsy, or cancer in animals. In an aspect, provided herein is a pharmaceutical composition comprising hemp extract and a carrier, wherein the hemp extract comprises:

cannabidiol; and

cannabidiolic acid;

wherein the ratio of cannabidiol to cannabidiolic acid is about 0.6:1 to about 1:0.6, and

wherein the carrier comprises biopolymer NF-971P and/or lecithin.

In another embodiment, the hemp extract comprises:

cannabidiol;

cannabidiolic acid;

cannabigerolic acid;

Δ9-tetrahydrocannabinol; and

cannabichromene;

wherein the ratio of cannabidiol to cannabidiolic acid is about 0.6:1 to about 1:0.6, and wherein the carrier comprises biopolymer NF-971P and/or lecithin.

In an embodiment, the hemp extract comprises:

α-pinene;

β-myrcene;

β-pinene;

δ-limonene;

linalool;

β-caryophyllene;

α-humulene;

nerolidol 2;

guaiol;

caryophyllene oxide; and

α-bisabolol.

In an embodiment the concentration of Δ9-tetrahydrocannabinol is insufficient to produce a psychotropic effect. In an embodiment, the ratio of Δ9-tetrahydrocannabinol to the other cannabinoids is about 1:25. In an embodiment, the concentration of Δ9-tetrahydrocannabinol is less than about 1 mg/mL. In another embodiment, the concentration of Δ9-tetrahydrocannabinol is less than about 0.5 mg/mL. In another embodiment, the concentration of Δ9-tetrahydrocannabinol is less than about 0.3 mg/mL. In another embodiment, the concentration of Δ9-tetrahydrocannabinol is less than about 0.2 mg/mL. In another embodiment, the concentration of Δ9-tetrahydrocannabinol is less than about 0.1 mg/mL. In another embodiment, the concentration of Δ9-tetrahydrocannabinol is about 0 mg/mL.

In an embodiment, the hemp extract comprises:

about 1-10 mg/mL of cannabidiol;

about 1-10 mg/mL of cannabidiolic acid;

about 0.05-0.2 mg/mL cannabigerolic acid;

about 0.1-0.3 mg/mL Δ9-tetrahydrocannabinol; and

about 0.1-0.4 mg/mL cannabichromene.

In another embodiment, the hemp extract comprises:

about 5 mg/mL of cannabidiol;

about 5 mg/mL of cannabidiolic acid;

about 0.11 mg/mL cannabigerolic acid;

about 0.25 mg/mL Δ9-tetrahydrocannabinol; and

about 0.27 mg/mL cannabichromene.

In an embodiment, the hemp extract comprises:

about 0.09-0.13% α-pinene;

about 0.23-0.44% β-myrcee;

about 0.04-0.09% β-pinene;

about 0.05-0.09% δ-limonene;

about 0.03-0.06% linalool;

about 0.04-0.07% β-caryophyllene;

about 0.02-0.04% α-humulene;

about 0.04-0.07% nerolidol 2;

about 0.02-0.04% guaiol;

about 0.04-0.08% caryophyllene oxide; and

about 0.01-0.04% α-bisabolol.

In an embodiment, the hemp extract further comprises:

camphene;

β-ocimene;

eucalyptol;

isopulegol; and/or

nerolidol 1.

In another embodiment, the hemp extract comprises:

about 0.02% camphene;

about 0.02-0.03% β-ocimene;

about 0.02-0.05% eucalyptol;

about 0.02% isopulegol; and/or

about 0.02-0.04% nerolidol 1.

In an embodiment, the composition is formulated in a carrier. In an embodiment, the carrier is selected from the group consisting of linseed oil, olive oil, fish oil, salmon oil, coconut oil, catnip oil, sesame oil, MCT oil, and grapeseed oil. In an embodiment, the carrier is grapeseed oil. In another embodiment, the carrier is catnip oil.

In an embodiment, the composition comprises lecithin. In an embodiment, the lecithin is sunflower lecithin. In an embodiment, the sunflower lecithin is up to 40%.

In an embodiment, the composition further comprises NF-971P. In an embodiment, the NF-971P is up to 2% weight/volume ratio.

In an embodiment, the hemp extract comprises:

α-pinene;

β-myrcene;

β-pinene;

δ-limonene;

linalool;

β-caryophyllene;

α-humulene;

nerolidol 2;

guaiol;

caryophyllene oxide; and

α-bisabolol and

one of biopolymer NF-971P and/or lecithin.

In an embodiment, the hemp extract comprises:

cannabidiol;

cannabidiolic acid;

cannabigerolic acid;

Δ9-tetrahydrocannabinol; and

cannabichromene;

wherein the carrier comprises grapeseed oil and one of biopolymer NF-971P and lecithin.

In an embodiment, the ratio of cannabidiol to cannabidiolic acid is selected from the group consisting of about 1:100, about 1:50, about 1:10, and about 1:1. In another embodiment, the ratio of cannabidiol to cannabidiolic acid is about 1:1.

In an embodiment, the hemp extract comprises cannabidiol and cannabidolic acid and includes no other components typically found in hemp oil. In some embodiments, this hemp extract does not include cannabigerolic acid, Δ9-tetrahydrocannabinol or cannabichromene. In some embodiments, this hemp extract does not include terpenes. In some embodiments, this hemp extract consists of cannabidiol and cannabidolic acid. In some embodiments, the ratio of cannabidiol to cannabidiolic acid is selected from the group consisting of about 1:100, about 1:50, about 1:10, and about 1:1. In another embodiment, the ratio of cannabidiol to cannabidiolic acid is about 1:1.

In some embodiments, the disclosure provides a purified pharmaceutical composition comprises cannabidiol and cannabidolic acid and includes no other components typically found in hemp oil. In some embodiments, the purified pharmaceutical composition does not include cannabigerolic acid, Δ9-tetrahydrocannabinol or cannabichromene. In some embodiments, the purified pharmaceutical composition does not include terpenes. In some embodiments, the purified pharmaceutical composition consists of cannabidiol and cannabidolic acid. In some embodiments, the ratio of cannabidiol to cannabidiolic acid is selected from the group consisting of about 1:100, about 1:50, about 1:10, and about 1:1. In another embodiment, the ratio of cannabidiol to cannabidiolic acid is about 1:1.

In an aspect, provided herein is a dosage form comprising a therapeutically effective amount of the hemp extract described above. In an embodiment, the dosage form comprises any of the pharmaceutical compositions described above and one or more pharmaceutically acceptable additives, flavoring agents, surfactants, and adjuvants. In an embodiment, the flavoring agent is selected from the group consisting of peppermint oil, mango extract, beef, poultry, and seafood.

In an embodiment, the dosage form is formulated as a sublingual spray. In another embodiment, the dosage form is formulated as a water or alcohol soluble solution, or a cream for transdermal application. In another embodiment, the dosage form is formulated as a gel for buccal or mucosal administration. In another embodiment, the dosage form is formulated as a powder. In another embodiment, the dosage form is formulated as a solution for subcutaneous injection. In another embodiment, the dosage form is formulated as a tablet. In another embodiment, the dosage form is formulated as a capsule. In another embodiment, the dosage form is formulated as a hard chewable. In another embodiment, the dosage form is formulated as a soft chewable. In another embodiment, the dosage form is formulated for administration using a nebulizer. In another embodiment, the dosage form is formulated for administration using a pet collar. In another embodiment, the dosage form is formulated for inhalation.

In an embodiment, the dosage form is formulated as a chew for oral administration. In an embodiment, the chew is produced using cold extrusion. In an embodiment, the weight of the chew is about 0.5-10 g. In another embodiment, the weight of the chew is about 4 g, about 6 g, about 9 g, or about 10 g. In an embodiment, the weight of the chew is about 4 g.

In an embodiment, the chew comprises:

about 7 mg of cannabidiol;

about 6 mg of cannabidiolic acid;

about 0.12 mg cannabigerolic acid;

about 0.32 mg Δ9-tetrahydrocannabinol; and

about 0.36 mg cannabichromene.

In an embodiment, the dosage form is formulated in a carrier for oral administration. In an embodiment, the carrier is selected from the group consisting of linseed oil, olive oil, fish oil, salmon oil, coconut oil, catnip oil, sesame oil, MCT oil, and grapeseed oil. In an embodiment, the carrier is grapeseed oil. In another embodiment, the carrier is catnip oil.

In an embodiment, the dosage form comprises:

glucosamine HCl;

chondroitin sulfate (76%);

brewer's yeast;

arabic gum;

guar gum;

a flavoring agent;

Verdilox;

Previon;

hemp extract;

lycerin;

sunflower lecithin; and

water.

In another embodiment, the dosage form comprises:

about 12-17% glucosamine HCl;

about 1-4% chondroitin sulfate (76%);

about 29-33% brewer's yeast;

about 3-6% arabic gum;

about 0.5-2% guar gum;

about 12-16% of a flavoring agent;

about 0.01-0.1% Verdilox;

about 0.5-1.5% Previon;

about 3-6% hemp extract;

about 13-17% glycerin;

about 3-7% sunflower lecithin; and

about 3-7% water.

In another embodiment, the dosage form comprises:

about 15.6% glucosamine HCl;

about 2.6% chondroitin sulfate (76%);

about 30% brewer's yeast;

about 4.7% arabic gum;

about 0.9% guar gum;

about 14.2% of a flavoring agent;

about 0.05% Verdilox;

about 0.9% Previon;

about 4.7% hemp extract;

about 15.1% glycerin;

about 5.7% sunflower lecithin; and

about 5.7% water.

In another embodiment, the dosage form comprises:

glucosamine HCl;

hyaluronic acid;

brewer's yeast;

arabic gum;

guar gum;

a flavoring agent;

Verdilox;

Previon;

hemp extract;

glycerin;

sunflower lecithin; and

water.

In another embodiment, the dosage form comprises:

about 12-17% glucosamine HCl;

about 0.01-1% hyaluronic acid;

about 29-33% brewer's yeast;

about 3-6% arabic gum;

about 0.5-2% guar gum;

about 12-16% of a flavoring agent;

about 0.01-0.1% Verdilox;

about 0.5-1.5% Previon;

about 3-6% hemp extract;

about 13-17% glycerin;

about 3-7% sunflower lecithin; and

about 3-7% water.

In an embodiment, the dosage form comprises:

about 16% glucosamine HCl;

about 0.1% hyaluronic acid;

about 30.6% brewer's yeast;

about 4.8% arabic gum;

about 0.97% guar gum;

about 14.5% of a flavoring agent;

about 0.05% Verdilox;

about 0.97% Previon;

about 4.8% hemp extract;

about 15.5% glycerin;

about 5.8% sunflower lecithin; and

about 5.8% water.

In another embodiment, the dosage form comprises:

hemp extract;

peanut butter;

rice bran;

glucosamine;

sweet potato;

molasses;

brewer's yeast;

sugar;

water;

glycerin; and

rice starch.

In an embodiment, the flavoring agent is selected from the group consisting of chicken liver powder, poultry extract, maltodextrin, butter, and bacon. In an embodiment the flavoring agent is chicken liver powder.

In an aspect, provided herein is a method for treating or reducing pain in a veterinary subject in need thereof, comprising administering to the subject a therapeutically effective amount of any of the compositions or dosage forms described above. In an embodiment, the pain is associated with arthritis, post-operative pain, acute pain, joint pain, or multi-joint pain.

In an aspect, provided herein is a method for treating epilepsy in a veterinary subject in need thereof, comprising administering to the subject a therapeutically effective amount of any of the compositions or dosage forms described above. In an embodiment, the method comprises treating epilepsy in a veterinary subject receiving phenobarbital, zonisamide, potassium bromide, and/or leviteracetam as anti-epileptic treatment, comprising administering to the subject a pharmaceutical composition or dosage form comprising about 2mg/kg per day of a hemp extract. In an embodiment, the veterinary subject is not receiving other anti-epileptic treatment. In an embodiment, the hemp extract is administered about every 12 hours. In another embodiment, the veterinary subject has previously experienced generalized motor seizures or focal seizure episodes. In another embodiment, the veterinary subject has a decrease in the frequency and/or duration of seizures.

In an embodiment, the hemp extract is present in a subject is a therapeutically effective amount of any of the compositions or dosage forms described above.

In an aspect, provided herein is a method for treating cancer in a veterinary subject in need thereof, comprising administering to the subject about 5 mg/kg of a hemp extract.

In an aspect, provided herein is a method for improving quality of life in a veterinary subject with cancer, comprising administering to the subject a therapeutically effective amount of any of the compositions or dosage forms described above.

In an embodiment, the veterinary subject is receiving L-CHOP or CHOP chemotherapy. In an embodiment, the hemp extract, composition or dosage form is administered about every 12 hours starting at week 4 or 5 of doxorubicin treatment.

In an embodiment, the cancer is lymphoma. In another embodiment, the lymphoma is intermediate to high-grade multicentric lymphoma. In another embodiment, following treatment the veterinary subject experiences an absence of lymphoma-associated abnormalities or a decrease in lymph node diameter.

In an embodiment, the veterinary subject has a body weight>15 kg.

In an embodiment, the veterinary subject is entering the end of the first cycle of L-CHOP chemotherapy.

In an aspect, provided herein is a method for treating post-operative pain in a veterinary subject in need thereof, comprising administering to the subject a therapeutically effective amount of any of the compositions or dosage forms described above.

In an aspect, provided herein is a method for treating post-operative pain in a veterinary subject in need thereof, comprising administering a hemp extract to the subject the night prior to surgery and then about every 12 hours for 12 days post-surgery along with a 5 day course of rimadyl.

In an embodiment, the veterinary subject has undergone tibial plateau leveling osteotomy surgery. In another embodiment, the veterinary subject has been treated with fentanyl and/or a nerve block.

In an embodiment, the pharmaceutical composition or dosage form is administered at a dosage of about 0.1-8.0 mg/kg.

In an embodiment, the pharmaceutical composition or dosage form is administered at twice the therapeutically effective dosage for one week, and then subsequently administered at a therapeutically effective dosage.

In an embodiment, the therapeutically effective dosage is about 0.1-0.5 mg/kg. In another embodiment, the therapeutically effective dosage is about 2 mg/kg. In another embodiment, the therapeutically effective dosage is about 8 mg/kg.

In an embodiment, the pharmaceutical composition or dosage form is administered at a dosage of about 1 mg/kg for one week, and then subsequently administered at a dosage of about 0.1-0.5 mg/kg.

In an embodiment, the pharmaceutical composition or dosage form is administered at a dosage of about 4 mg/kg for one week, and then subsequently administered at a dosage of about 2 mg/kg.

In an embodiment, the method results in a therapeutically effective median maximal serum concentration of cannabidiol. In an embodiment, the median maximal serum concentration of cannabidiol is about 102 ng/mL. In an embodiment, the median maximal serum concentration of cannabidiol is about 590 ng/mL.

In an embodiment, the veterinary subject is canine, feline, bovine, porcine, or equine. In an embodiment, the veterinary subject is canine. In another embodiment, the veterinary subject is feline.

In an aspect, provided herein is a method of achieving an area under the curve from 0 time to 24 hours of between 42.4 and 3048 ng hr/ml for cannabidiol in a veterinary subject comprising administering to the subject an effective amount of hemp extract.

In an aspect, provided herein is a method of treating or reducing pain in a veterinary subject in need thereof, comprising administering a therapeutically effective amount of a pharmaceutical composition comprising hemp extract and a carrier, wherein the hemp extract comprises:

cannabidiol; and

cannabidiolic acid;

wherein the ratio of cannabidiol to cannabidiolic acid is about 0.6:1 to about 1:0.6.

In an aspect, provided herein is a method of treating epilepsy in a veterinary subject in need thereof, comprising administering a therapeutically effective amount of a pharmaceutical composition comprising hemp extract and a carrier, wherein the hemp extract comprises:

cannabidiol; and

cannabidiolic acid;

wherein the ratio of cannabidiol to cannabidiolic acid is about 0.6:1 to about 1:0.6.

In an aspect, provided herein is a method of treating cancer in a veterinary subject in need thereof, comprising administering a therapeutically effective amount of a pharmaceutical composition comprising hemp extract and a carrier, wherein the hemp extract comprises:

cannabidiol; and

cannabidiolic acid;

wherein the ratio of cannabidiol to cannabidiolic acid is about 0.6:1 to about 1:0.6.

In an aspect, provided herein is a method of improving quality of life in a veterinary subject with cancer, comprising administering a therapeutically effective amount of a pharmaceutical composition comprising hemp extract and a carrier, wherein the hemp extract comprises:

cannabidiol; and

cannabidiolic acid;

wherein the ratio of cannabidiol to cannabidiolic acid is about 0.6:1 to about 1:0.6.

In an embodiment, the hemp extract further comprises:

cannabigerolic acid;

Δ9-tetrahydrocannabinol; and

cannabichromene;

In another embodiment, the hemp extract further comprises four or more of the following:

α-pinene;

β-myrcene;

β-pinene;

δ-limonene;

linalool;

β-caryophyllene;

α-humulene;

nerolidol 2;

guaiol;

caryophyllene oxide; and

α-bisabolol.

In another embodiment, the concentration of Δ9-tetrahydrocannabinol is insufficient to produce a psychotropic effect. In another embodiment, the ratio of Δ9-tetrahydrocannabinol to the other cannabinoids is about 1:25. In another embodiment, the concentration of Δ9-tetrahydrocannabinol is less than about 1 mg/mL. In another embodiment, the concentration of Δ9-tetrahydrocannabinol is less than about 0.5 mg/mL. In another embodiment, the concentration of Δ9-tetrahydrocannabinol is less than about 0.3 mg/mL. In another embodiment, the concentration of Δ9-tetrahydrocannabinol is less than about 0.2 mg/mL. In another embodiment, the concentration of Δ9-tetrahydrocannabinol is less than about 0.1 mg/mL. In another embodiment, the concentration of Δ9-tetrahydrocannabinol is about 0 mg/mL.

In an embodiment, the hemp extract comprises:

about 1-10 mg/mL of cannabidiol;

about 1-10 mg/mL of cannabidiolic acid;

about 0.05-0.2 mg/mL cannabigerolic acid;

about 0.1-0.3 mg/mL Δ9-tetrahydrocannabinol; and

about 0.1-0.4 mg/mL cannabichromene.

In another embodiment, the hemp extract comprises:

about 5 mg/mL of cannabidiol;

about 5 mg/mL of cannabidiolic acid;

about 0.11 mg/mL cannabigerolic acid;

about 0.25 mg/mL Δ9-tetrahydrocannabinol; and

about 0.27 mg/mL cannabichromene.

In another embodiment, the hemp extract comprises:

about 0.09-0.13% α-pinene;

about 0.23-0.44% β-myrcene;

about 0.04-0.09% β-pinene;

about 0.05-0.09% δ-limonene;

about 0.03-0.06% linalool;

about 0.04-0.07% β-caryophyllene;

about 0.02-0.04% α-humulene;

about 0.04-0.07% nerolidol 2;

about 0.02-0.04% guaiol;

about 0.04-0.08% caryophyllene oxide; and

about 0.01-0.04% α-bisabolol.

In an embodiment, the hemp extract further comprises:

camphene;

β-ocimene;

eucalyptol;

isopulegol; and/or

nerolidol 1.

In another embodiment, the hemp extract comprises:

about 0.02% camphene;

about 0.02-0.03% β-ocimene;

about 0.02-0.05% eucalyptol;

about 0.02% isopulegol; and/or

about 0.02-0.04% nerolidol 1.

In an embodiment, wherein the hemp extract comprises 5, 6, 7, 8, 9, 10, 11, 12, 13, 14, 15 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the composition is formulated in a carrier. In another embodiment, the carrier is selected from the group consisting of linseed oil, olive oil, fish oil, salmon oil, coconut oil, catnip oil, sesame oil, MCT oil, and grapeseed oil. In another embodiment, the carrier is grapeseed oil. In another embodiment, the carrier is catnip oil.

In an embodiment, the composition comprises nepetalactone. In another embodiment, the composition comprises taurine.

In an embodiment, the composition is formulated for administration using a nebulizer. In another embodiment, the composition is formulated for administration using a diffuser. In another embodiment, the composition is formulated for administration using a pet collar. In another embodiment, the composition is formulated as a pet food for oral administration.

In an embodiment, the composition is formulated as a chew for oral administration. In another embodiment, the weight of the chew is about 0.5-10 g. In another embodiment, the weight of the chew is about 4 g, about 6 g, about 9 g, or about 10 g. In another embodiment, the weight of the chew is about 4 g.

In another embodiment, the chew comprises:

about 7 mg of cannabidiol;

about 6 mg of cannabidiolic acid;

about 0.12 mg cannabigerolic acid;

about 0.32 mg Δ9-tetrahydrocannabinol; and

about 0.36 mg cannabichromene.

In an aspect, provided herein is a method of treating or reducing pain in a veterinary subject in need thereof, comprising administering a therapeutically effective amount of a pharmaceutical composition comprising hemp extract and a carrier, wherein the hemp extract comprises four or more of:

α-pinene;

β-myrcene;

β-pinene;

δ-limonene;

linalool;

β-caryophyllene;

α-humulene;

nerolidol 2;

guaiol;

caryophyllene oxide; and

α-bisabolol.

In an aspect, provided herein is method of treating epilepsy in a veterinary subject in need thereof, comprising administering a therapeutically effective amount of a pharmaceutical composition comprising hemp extract and a carrier, wherein the hemp extract comprises four or more of:

α-pinene;

β-myrcene;

β-pinene;

δ-limonene;

linalool;

β-caryophyllene;

α-humulene;

nerolidol 2;

guaiol;

caryophyllene oxide; and

α-bisabolol.

In an aspect, provided herein is method of treating cancer in a veterinary subject in need thereof, comprising administering a therapeutically effective amount of a pharmaceutical composition comprising hemp extract and a carrier, wherein the hemp extract comprises four or more of:

α-pinene;

β-myrcene;

β-pinene;

δ-limonene;

linalool;

β-caryophyllene;

α-humulene;

nerolidol 2;

guaiol;

caryophyllene oxide; and

α-bisabolol.

In an aspect, provided herein is method of improving quality of life in a veterinary subject with cancer, comprising administering a therapeutically effective amount of a pharmaceutical composition comprising hemp extract and a carrier, wherein the hemp extract comprises four or more of:

α-pinene;

β-myrcene;

β-pinene;

δ-limonene;

linalool;

β-caryophyllene;

α-humulene;

nerolidol 2;

guaiol;

caryophyllene oxide; and

α-bisabolol.

In an embodiment the hemp extract comprises:

about 0.09-0.13% α-pinene;

about 0.23-0.44% β-myrcene;

about 0.04-0.09% β-pinene;

about 0.05-0.09% δ-limonene;

about 0.03-0.06% linalool;

about 0.04-0.07% β-caryophyllene;

about 0.02-0.04% α-humulene;

about 0.04-0.07% nerolidol 2;

about 0.02-0.04% guaiol;

about 0.04-0.08% caryophyllene oxide; and

about 0.01-0.04% α-bisabolol.

In another embodiment, the hemp extract further comprises:

camphene;

β-ocimene;

eucalyptol;

isopulegol; and/or

nerolidol 1.

In another embodiment, the hemp extract comprises:

about 0.02% camphene;

about 0.02-0.03% β-ocimene;

about 0.02-0.05% eucalyptol;

about 0.02% isopulegol; and/or

about 0.02-0.04% nerolidol 1.

In an embodiment, the hemp extract comprises 5, 6, 7, 8, 9, 10, 11, 12, 13, 14, 15 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the composition is formulated in a carrier. In another embodiment, the carrier is selected from the group consisting of linseed oil, olive oil, fish oil, salmon oil, coconut oil, catnip oil, sesame oil, MCT oil, and grapeseed oil. In another embodiment, the carrier is grapeseed oil. In another embodiment, the carrier is catnip oil.

In an embodiment, the composition comprises nepetalactone. In another embodiment, the composition comprises taurine.

In an embodiment, the composition is formulated for administration using a nebulizer. In another embodiment, the composition is formulated for administration using a diffuser. In another embodiment, the composition is formulated for administration using a pet collar.

In an embodiment, the composition is formulated as a chew for oral administration. In another embodiment, the weight of the chew is about 0.5-10 g. In another embodiment, the weight of the chew is about 4 g, about 6 g, about 9 g, or about 10 g. In another embodiment, the weight of the chew is about 4 g.

In an aspect, provided herein is a method of treating or reducing pain in a veterinary subject in need thereof, comprising administering a dosage form comprising:

cannabidiol;

cannabidiolic acid;

cannabigerolic acid;

Δ9-tetrahydrocannabinol;

cannabichromene; and

one or more pharmaceutically acceptable additives, flavoring agents, surfactants, and adjuvants.

In an aspect, provided herein is a method of treating epilepsy in a veterinary subject in need thereof, comprising administering a dosage form comprising:

cannabidiol;

cannabidiolic acid;

cannabigerolic acid;

Δ9-tetrahydrocannabinol;

cannabichromene; and

one or more pharmaceutically acceptable additives, flavoring agents, surfactants, and adjuvants.

In an aspect, provided herein is a method of treating cancer in a veterinary subject in need thereof, comprising administering a dosage form comprising:

cannabidiol;

cannabidiolic acid;

cannabigerolic acid;

Δ9-tetrahydrocannabinol;

cannabichromene; and

one or more pharmaceutically acceptable additives, flavoring agents, surfactants, and adjuvants.

In an aspect, provided herein is a method of improving quality of life in a veterinary subject with cancer, comprising administering a dosage form comprising:

cannabidiol;

cannabidiolic acid;

cannabigerolic acid;

Δ9-tetrahydrocannabinol;

cannabichromene; and

one or more pharmaceutically acceptable additives, flavoring agents, surfactants, and adjuvants.

In an embodiment, the ratio of cannabidiol to cannabidiolic acid is selected from the group consisting of about 1:100, about 1:50, about 1:10, and about 1:1. In another embodiment, the ratio of cannabidiol to cannabidiolic acid is about 1:1. In another embodiment, the concentration of Δ9-tetrahydrocannabinol is insufficient to produce a psychotropic effect. In another embodiment, the ratio of Δ9-tetrahydrocannabinol to the other cannabinoids is about 1:25. In another embodiment, the concentration of Δ9-tetrahydrocannabinol is less than about 1 mg/mL. In another embodiment, the concentration of Δ9-tetrahydrocannabinol is less than about 0.5 mg/mL. In another embodiment, the concentration of Δ9-tetrahydrocannabinol is less than about 0.3 mg/mL. In another embodiment, the concentration of Δ9-tetrahydrocannabinol is less than about 0.2 mg/mL. In another embodiment, the concentration of Δ9-tetrahydrocannabinol is less than about 0.1 mg/mL. In another embodiment, the concentration of Δ9-tetrahydrocannabinol is about 0 mg/mL.

In an embodiment, the hemp extract comprises:

about 1-10 mg/mL of cannabidiol;

about 1-10 mg/mL of cannabidiolic acid;

about 0.05-0.2 mg/mL cannabigerolic acid;

about 0.1-0.3 mg/mL Δ9-tetrahydrocannabinol; and

about 0.1-0.4 mg/mL cannabichromene.

In another embodiment, the hemp extract comprises:

about 5 mg/mL of cannabidiol;

about 5 mg/mL of cannabidiolic acid;

about 0.11 mg/mL cannabigerolic acid;

about 0.25 mg/mL Δ9-tetrahydrocannabinol; and

about 0.27 mg/mL cannabichromene.

In an embodiment, the hemp extract comprises four or more of the following:

α-pinene;

β-myrcene;

β-pinene;

δ-limonene;

linalool;

β-caryophyllene;

α-humulene;

nerolidol 2;

guaiol;

caryophyllene oxide; and

α-bisabolol.

In another embodiment, the hemp extract comprises:

about 0.09-0.13% α-pinene;

about 0.23-0.44% β-myrcene;

about 0.04-0.09% β-pinene;

about 0.05-0.09% δ-limonene;

about 0.03-0.06% linalool;

about 0.04-0.07% β-caryophyllene;

about 0.02-0.04% α-humulene;

about 0.04-0.07% nerolidol 2;

about 0.02-0.04% guaiol;

about 0.04-0.08% caryophyllene oxide; and

about 0.01-0.04% α-bisabolol.

In another embodiment, the hemp extract comprises:

camphene;

β-ocimene;

eucalyptol;

isopulegol; and/or

nerolidol 1.

In another embodiment, the hemp extract comprises:

about 0.02% camphene;

about 0.02-0.03% β-ocimene;

about 0.02-0.05% eucalyptol;

about 0.02% isopulegol; and/or

about 0.02-0.04% nerolidol 1.

In an embodiment, the hemp extract comprises 5, 6, 7, 8, 9, 10, 11, 12, 13, 14, 15 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the flavoring agent is selected from the group consisting of catnip oil, peppermint oil, mango extract, beef, poultry, and seafood. In an embodiment, the flavoring agent is catnip oil.

In an embodiment, the dosage form comprises nepetalactone. In another embodiment, the dosage form comprises taurine.

In an embodiment, the dosage form is formulated as a chew for oral administration. In another embodiment, the chew is produced using cold extrusion. In another embodiment, the dosage form is formulated as a sublingual spray. In another embodiment, the dosage form is formulated as a water or alcohol soluble solution, a gel, or a cream for transdermal application. In another embodiment, the dosage form is formulated as a gel for buccal or mucosal administration. In another embodiment, the dosage form is formulated as a powder. In another embodiment, the dosage form is formulated as a solution for subcutaneous injection. In another embodiment, the dosage form is formulated as a tablet. In another embodiment, the dosage form is formulated as a capsule. In another embodiment, the dosage form is formulated as a hard chewable. In another embodiment, the dosage form is formulated as a soft chewable.

In an embodiment, the dosage form is formulated in a carrier for oral administration. In another embodiment, the carrier is selected from the group consisting of linseed oil, olive oil, fish oil, salmon oil, coconut oil, catnip oil, sesame oil, MCT oil, and grapeseed oil. In another embodiment, the dosage form is the carrier is grapeseed oil. In another embodiment, the dosage form is the carrier is catnip oil.

In another embodiment, the dosage form is formulated for inhalation. In another embodiment, the dosage form is formulated for administration using a nebulizer. In another embodiment, the dosage form is formulated for administration using a diffuser. In another embodiment, the dosage form is formulated for administration using a pet collar.

In an aspect, provided herein is a method of treating or reducing pain in a veterinary subject in need thereof, comprising administering a dosage form comprising:

glucosamine HCl;

chondroitin sulfate (76%);

brewer's yeast;

arabic gum;

guar gum;

a flavoring agent;

Verdilox;

Previon;

hemp extract;

glycerin;

sunflower lecithin; and

water.

In an aspect, provided herein is a method of treating epilepsy in a veterinary subject in need thereof, comprising administering a dosage form comprising:

glucosamine HCl;

chondroitin sulfate (76%);

brewer's yeast;

arabic gum;

guar gum;

a flavoring agent;

Verdilox;

Previon;

hemp extract;

glycerin;

sunflower lecithin; and

water.

In an aspect, provided herein is a method of treating cancer in a veterinary subject in need thereof, comprising administering a dosage form comprising:

glucosamine HCl;

chondroitin sulfate (76%);

brewer's yeast;

arabic gum;

guar gum;

a flavoring agent;

Verdilox;

Previon;

hemp extract;

glycerin;

sunflower lecithin; and

water.

In an aspect, provided herein is a method of improving quality of life in a veterinary subject with cancer, comprising administering a dosage form comprising:

glucosamine HCl;

chondroitin sulfate (76%);

brewer's yeast;

arabic gum;

guar gum;

a flavoring agent;

Verdilox;

Previon;

hemp extract;

glycerin;

sunflower lecithin; and

water.

In an embodiment, the dosage form comprises:

about 12-17% glucosamine HCl;

about 1-4% chondroitin sulfate (76%);

about 29-33% brewer's yeast;

about 3-6% arabic gum;

about 0.5-2% guar gum;

about 12-16% of a flavoring agent;

about 0.01-0.1% Verdilox;

about 0.5-1.5% Previon;

about 3-6% hemp extract;

about 13-17% glycerin;

about 3-7% sunflower lecithin; and

about 3-7% water.

In another embodiment, the dosage form comprises:

about 15.6% glucosamine HCl;

about 2.6% chondroitin sulfate (76%);

about 30% brewer's yeast;

about 4.7% arabic gum;

about 0.9% guar gum;

about 14.2% of a flavoring agent;

about 0.05% Verdilox;

about 0.9% Previon;

about 4.7% hemp extract;

about 15.1% glycerin;

about 5.7% sunflower lecithin; and

about 5.7% water.

In an aspect, provided herein is a method of treating or reducing pain in a veterinary subject in need thereof, comprising administering a dosage form comprising:

glucosamine HCl;

hyaluronic acid;

brewer's yeast;

arabic gum;

guar gum;

a flavoring agent;

Verdilox;

Previon;

hemp extract;

glycerin;

sunflower lecithin; and

water.

In an aspect, provided herein is a method of treating epilepsy in a veterinary subject in need thereof, comprising administering a dosage form comprising:

glucosamine HCl;

hyaluronic acid;

brewer's yeast;

arabic gum;

guar gum;

a flavoring agent;

Verdilox;

Previon;

hemp extract;

glycerin;

sunflower lecithin; and

water.

In an aspect, provided herein is a method of treating cancer in a veterinary subject in need thereof, comprising administering a dosage form comprising:

glucosamine HCl;

hyaluronic acid;

brewer's yeast;

arabic gum;

guar gum;

a flavoring agent;

Verdilox;

Previon;

hemp extract;

glycerin;

sunflower lecithin; and

water.

In an aspect, provided herein is a method of improving quality of life in a veterinary subject in need thereof, comprising administering a dosage form comprising:

glucosamine HCl;

hyaluronic acid;

brewer's yeast;

arabic gum;

guar gum;

a flavoring agent;

Verdilox;

Previon;

hemp extract;

glycerin;

sunflower lecithin; and

water.

In an embodiment, the dosage form comprises:

about 12-17% glucosamine HCl;

about 0.01-1% hyaluronic acid;

about 29-33% brewer's yeast;

about 3-6% arabic gum;

about 0.5-2% guar gum;

about 12-16% of a flavoring agent;

about 0.01-0.1% Verdilox;

about 0.5-1.5% Previon;

about 3-6% hemp extract;

about 13-17% glycerin;

about 3-7% sunflower lecithin; and

about 3-7% water.

In another embodiment, the dosage form comprises:

about 16% glucosamine HCl;

about 0.1% hyaluronic acid;

about 30.6% brewer's yeast;

about 4.8% arabic gum;

about 0.97% guar gum;

about 14.5% of a flavoring agent;

about 0.05% Verdilox;

about 0.97% Previon;

about 4.8% hemp extract;

about 15.5% glycerin;

about 5.8% sunflower lecithin; and

about 5.8% water.

In an aspect, provided herein is a method of treating or reducing pain in a veterinary subject in need thereof, comprising administering a dosage form comprising:

hemp extract;

peanut butter;

rice bran;

glucosamine HCL;

sweet potato;

dry molasses;

sorbic acid

brewer's yeast;

sugar;

water;

glycerin;

potato starch;

dehydrated peanut butter;

rice starch; and

guar gum.

In an aspect, provided herein is a method of treating epilepsy in a veterinary subject in need thereof, comprising administering a dosage form comprising:

hemp extract;

peanut butter;

rice bran;

glucosamine HCL;

sweet potato;

dry molasses;

sorbic acid

brewer's yeast;

sugar;

water;

glycerin;

potato starch;

dehydrated peanut butter;

rice starch; and

guar gum.

In an aspect, provided herein is a method of treating cancer in a veterinary subject in need thereof, comprising administering a dosage form comprising:

hemp extract;

peanut butter;

rice bran;

glucosamine HCL;

sweet potato;

dry molasses;

sorbic acid

brewer's yeast;

sugar;

water;

glycerin;

potato starch;

dehydrated peanut butter;

rice starch; and

guar gum.

In an aspect, provided herein is a method of improving quality of life in a veterinary subject in need thereof, comprising administering a dosage form comprising:

hemp extract;

peanut butter;

rice bran;

glucosamine HCL;

sweet potato;

dry molasses;

sorbic acid

brewer's yeast;

sugar;

water;

glycerin;

potato starch;

dehydrated peanut butter;

rice starch; and

guar gum.

In an embodiment, the dosage from comprises:

about 5.0% hemp extract;

about 15.0% peanut butter;

about 12.5% rice bran;

about 12.75% glucosamine HCL;

about 5.5% sweet potato;

about 8.0% dry molasses;

about 1% sorbic acid;

about 5.0% brewer's yeast;

about 6.0% sugar;

about 9.25% water;

about 13.0 glycerin;

about 2.0% potato starch;

about 1.0% dehydrated peanut butter;

about 2.0% rice starch; and

about 2.0% guar gum.

In an embodiment, the dosage from comprises:

about 5.0% hemp extract;

about 15.0% peanut butter;

about 13.0% rice bran;

about 8.5% glucosamine HCL;

about 6.0% sweet potato;

about 9.0% dry molasses;

about 1% sorbic acid;

about 5.0% brewer's yeast;

about 6.0% sugar;

about 9.5% water;

about 13.0 glycerin;

about 4.0% potato starch;

about 1.0% dehydrated peanut butter;

about 2.0% rice starch; and

about 2.0% guar gum.

In an embodiment, the dosage from comprises:

about 3.0-10.0% hemp extract;

about 10.0-20.0% peanut butter;

about 10.0-15.0% rice bran;

about 5.0-15.0% glucosamine HCL;

about 4.0-10.0% sweet potato;

about 6.0-13.0% dry molasses;

about 0.5-5.0% sorbic acid;

about 2.0-8.0% brewer's yeast;

about 3.0-8.0% sugar;

about 5.0-15.0% water;

about 8.0-18.0% glycerin;

about 1.0-8.0% potato starch;

about 0.5-5.0% dehydrated peanut butter;

about 1.0-5.0% rice starch; and

about 1.0-5.0% guar gum.

In an embodiment, the dosage form comprises about 5.0% hemp extract.

In an embodiment, the flavoring agent is selected from the group consisting of catnip oil, chicken liver powder, poultry extract, maltodextrin, butter, and bacon. In another embodiment the flavoring agent is chicken liver powder. In another embodiment, the flavoring agent is catnip oil.

In an embodiment, the dosage form comprises nepetalactone. In another embodiment, the dosage form comprises taurine.

In an embodiment, the dosage form is formulated as a chew for oral administration. In an embodiment, the chew is produced using cold extrusion.

In an embodiment, the veterinary subject is treated for pain using a method described above in combination with gabapentin. In an embodiment, the pain is post-operative. In another embodiment, the veterinary subject has undergone spinal surgery. In another embodiment, the spinal surgery is a hemilaminectomy. In an embodiment, the veterinary subject is administered 8-12 mg/kg of gabapentin and 6-10 mg/kg of hemp extract every 6-10 hours. In another embodiment, the veterinary subject is administered about 10 mg/kg gabapentin and about 8 mg/kg hemp extract every 8 hours.

### BRIEF DESCRIPTION OF THE FIGURES

FIG. 1: Box-and-whisker plot of serum alkaline phosphatase (ALP) activity at each time for treatment and placebo oils. Box represents the mean and 25th and 75th percentile and the whiskers represent the 99th and 1st percentiles.

FIG. 2: Serum concentration (ng/mL) of 2 mg/kg and 8 mg/kg oral dosage of CBD oil in time (min)

FIG. 3A: Box-and-whisker plot of total CBPI score at each time for treatment and placebo oils. Box represents the mean and 25th and 75th percentile and the whiskers represent the 99th and 1st percentiles.

FIG. 3B: Box-and-whisker plot of total Hudson score at each time for treatment and placebo oils. Box represents the mean and 25th and 75th percentile and the whiskers represent the 99th and 1st percentiles.

FIG. 4: Box-and-whisker plot of total vet pain assessment at each time for treatment and placebo oils.

FIGS. 5A-5F: Graphs showing trot stance % gait cycle symmetry (FIG. 5A), trot stance % gait cycle (FIG. 5B), trot step/stride ratio (FIG. 5C), walk stance % gait cycle symmetry (FIG. 5D), walk stance % gait cycle (FIG. 5E), and walk step stride ratio (FIG. 5F) for five dogs treated with CBD oil.

FIG. 6: Graph showing % viability of cancer cell lines treated with CBD oil.

FIGS. 7A and 7B: Graph and blot showing caspase activity as a marker of apoptosis in cancer cell lines treated with CBD oil. Caspase activity was measured by fold increase in luminescence using Apo-glo (FIG. 7A) and by western blot of caspase (FIG. 7B).

FIG. 8: Plot of combination index of CBD oil and doxorubicin on D17 cells.

FIG. 9: Graph showing % viability of cancer cell lines treated with doxorubicin.

FIGS. 10A-10D: Graphs showing serum concentrations of CBD (FIG. 10A), CBDA (FIG. 10B), THC (FIG. 10C), and THCA (FIG. 10D) in dogs treated with Oil A, Oil B, and chewable CBD formulations. Bars marked with an asterisk represent a significant difference at that time point.

### DETAILED DESCRIPTION

The endocannabinoid receptor system is known to play a role in pain modulation and attenuation of inflammation. Cannabinoid receptors (CB1 and CB2) are widely distributed throughout the central and peripheral nervous system and are also present in the synovium. However, the psychotropic effects of certain cannabinoids prevent extensive research into their use as single agents for pain relief. The cannabinoids are a group of as many as 60 different compounds that may or may not act at CB receptors. One class of cannabinoids, cannabidiol (CBD), may actually be an antagonist of the CB receptors. In lower vertebrates, CBD can also have immunomodulatory, anti-hyperalgesic, antinociceptive, and anti-inflammatory actions, making it an attractive therapeutic option in dogs with OA cancer, epilepsy, or that have post-operative pain.

### Cancer

Lymphoma is one of the most commonly diagnosed hematopoietic cancers in dogs. The most common chemotherapy protocol used for this disease is a doxorubicin-based multidrug protocol (L-asparaginase, cyclophosphamide, doxorubicin, vincristine, and prednisone). Remission rates for such protocols range from 80-90%. Median survival time in dogs diagnosed with lymphoma treated with a doxorubicin based chemotherapy protocol ranges from 6 to 12 months. Approximately 20% to up to 50% of dogs undergoing CHOP or L-CHOP chemotherapy may experience variable degree of GI toxicity (Tomiyasu et al. (2010) J Vet Med Sci. 72(11):1391-7.; Mason et al. (2014) J Small Anim Pract. 55(8):391-8.). The availability of intensive therapeutic modalities and increased client willingness to pursue treatment has led to improved survival times in veterinary cancer patients. However, with more intensive therapy, the risk of associated morbidity has also increased and balancing quality of life with quantity of life has become an important role for the veterinary oncologist. Owners of pets with terminal illness tend to value quality of life (QoL) over longevity and are willing to trade survival time to preserve QoL (Iliopoulou et al. (2013) J Am Vet Med Assoc. 242(12):1679-87.; Giuffrida et al. (2018) J Amer Vet Med Assoc. 252:1073-1083.).

The use of hemp products, including marijuana and cannabidiol (CBD), for medical conditions are becoming much more common for both people and pets. Cannabidiol (CBD) is chemically and phytogenetically related to phenolic terpenes derived from hemp. CBD oil is proposed to have many beneficial health effects that could translate into an improved QoL.

A recent survey by the American Holistic Veterinary Medical Association revealed that almost 60% of people who buy hemp products online use these products for their dogs. Industrial hemp products that are low in THC (0.3%) and higher in other cannabinoids are reported to have health benefits including analgesic, anti-inflammatory, anti-anxiolytic, and anti-epileptic; and are legal according to the industrial hemp act. There are numerous on-line companies selling hemp products including CBD oil claiming they are safe and effective for various medical conditions in both pets and people. There is very little published data to support these claims and no data to indicate the safety of using CBD oil concurrently with chemotherapy in veterinary patients.

No clinical study has specifically studied the risk of interaction between cannabinoids and anticancer agents. As any drug metabolized through the liver, CBD may exhibit inhibition of metabolism of other drugs (Bouque et al. (2018) Fund Clin Pharm. 32:462-484). In addition, it may alter the distribution of drugs through transmembrane pump interactions. Therefore, there is a need to assess the safety and tolerability of CBD oil use during chemotherapy for lymphoma and its effect on health related quality of life (HRQL).

### Epilepsy

The use of cannabinoid derivatives in the treatment of a variety of neurological disorders in humans has recently been explored, particularly in the treatment of chronic pain and epilepsy. Full spectrum cannabinoid rich industrial hemp products below 0.3% THC have been shown to have no psychotropic effects and modest activity through non-cannabinoid receptor routes affecting the serotonergic, glycinergic and GABAergic neurotransmission pathways. Recent research using full spectrum cannabinoid rich HBNs has revealed efficacy of these products in dogs with chronic pain (Wakshlag et al., Front Vet Sci, 2018). Additionally, investigation in epilepsy in humans and the release of Epidiolex as viable treatment shows merits to cannabidiol in the treatment of epilepsy, but there have been no published investigations evaluating efficacy of HBNs in a canine epilepsy model. There is a need to evaluate whether treatment of refractory canine epilepsy with a HBN will decrease seizure numbers or duration and whether it will alter metabolism of other commonly used drugs for seizure control in dogs.

### Post-Operative Pain Relief

The use of cannabinoid derivatives in the treatment of a variety of neurological disorders in humans has recently been explored, particularly in the treatment of chronic pain and epilepsy. Full spectrum cannabinoid rich Industrial hemp based nutraceuticals (HBN) below 0.3% THC have been shown to have no psychotropic effects and modest activity through non-cannabinoid receptor routes affecting the serotonergic, glycinergic and GABA neurotransmission pathways that may be able to diminish pain, as well as inflammation.

Recent research into chronic pain has revealed efficacy of these. HBN in diminishing chronic osteoarthritic pain based on objective client-based validated surveys, however post-surgical pain relief is still unexamined. The use of a HBN should help alleviate post-operative pain and improve outcomes in dogs undergoing tibial plateau leveling osteotomy surgery for cranial cruciate rupture.

In addition, cannabinoids may be useful in alleviating post-operative pain following spinal cord injury and hemilaminectomy. Post-operative pain following spinal cord injury and hemilaminectomy is complex and involves inflammatory nociceptive and neuropathic mechanisms of pain. The complexity of post-operative hemilaminectomy pain necessitates a multimodal analgesia protocol. Current standard of care in post-operative hemilaminectomy patients is parenteral opioids with protocols for adjunctive analgesics varying by institution. While effective in controlling post-operative pain, opioid use can also be associated with undesirable adverse effects such as: vomiting, inappetence, dysphoria, central nervous system and respiratory depression, constipation and sedation. The goal of adjunctive analgesia in post-operative hemilaminectomy patients is two-fold, to decrease the amount of opioids needed for pain control and to target multiple mechanisms of pain. Typical adjunctive analgesic medications may include gabapentin, diazepam, and non-steroidal anti-inflammatory or steroid medications. Gabapentin, a gold-standard for neuropathic pain, is generally well tolerated by patients and is commonly used in post-operative neurosurgery patients. An alternative therapeutic treatment that shows potential is cannabidiol. Gabapentin and cannabidiol combination treatment provides relief from post-operative pain following spinal cord injury and hemilaminectomy.

The present disclosure is directed toward compositions comprising hemp extract and their use for the treatment of pain in animals. Also provided herein are methods for treatment of pain in veterinary subjects. The efficacy of these compositions and treatment methods has not previously been demonstrated. Clinical trial and pharmacokinetic data regarding dosing is also provided herein.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±5%, from the specified value, as such variations are appropriate to perform the disclosed methods.

As used in the specification and in the claims, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “treatment” or “treating,” is defined as the application or administration of a therapeutic agent, i.e., a compound provided herein (alone or in combination with another pharmaceutical agent), to a patient, or application or administration of a therapeutic agent to an isolated tissue or cell line from a patient (e.g., for diagnosis or ex vivo applications), with the purpose to cure, heal, alleviate, relieve, alter, remedy, ameliorate, improve or affect the symptoms of a disease, disorder, syndrome, or condition. Such treatments can be specifically tailored or modified, based on knowledge obtained from the field of pharmacogenomics.

In certain embodiments, the compositions described herein reduce pain in a subject. Pain can be measured using any metric known in the art. For example, pain can be measured using the canine brief pain inventory (CBPI), the Hudson activity scale, flexion and tension measurements and gait analysis. A reduction in any of these metrics shows a treatment of or reduction in pain.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “use” includes any one or more of the following embodiments of the invention, respectively: the use in the treatment of pain the use for the manufacture of pharmaceutical compositions for use in the treatment of these diseases, e.g., in the manufacture of a medicament; methods of use of compounds of the invention in the treatment of these diseases; pharmaceutical preparations having compounds of the invention for the treatment of these diseases; and compounds of the invention for use in the treatment of these diseases; as appropriate and expedient, if not stated otherwise.

As used herein, the term “patient,” “individual,” or “subject” is intended to include organisms, e.g., prokaryotes and eukaryotes, which are capable of suffering from or afflicted with a disease, disorder or condition associated with the activity of a protein kinase. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from, schizophrenia. In another embodiment, the subject is a cell.

When used with respect to methods of treatment/prevention and the use of the compounds and pharmaceutical compositions thereof described herein, an individual “in need thereof” may be an individual who has been diagnosed with or previously treated for the condition to be treated. With respect to prevention, the individual in need thereof may also be an individual who is at risk for a condition (e.g., a family history of the condition, lifestyle factors indicative of risk for the condition, etc.). Typically, when a step of administering a compound of the invention is disclosed herein, the invention further contemplates a step of identifying an individual or subject in need of the particular treatment to be administered or having the particular condition to be treated.

In some embodiments, the individual is a mammal, including, but not limited to, bovine, equine, feline, rabbit, canine, rodent, or primate. In some embodiments, the mammal is a primate. In some embodiments, the primate is a human. In some embodiments, the individual is human, including adults, children and premature infants. In some embodiments, the individual is a non-mammal. In some variations, the primate is a non-human primate such as chimpanzees and other apes and monkey species. The term “individual” does not denote a particular age or sex.

As used herein, the term “pharmaceutically acceptable” refers to a material, such as a carrier or diluent, which does not abrogate the biological activity or properties of the compound, and is relatively non-toxic, i.e., the material can be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” or “carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it can perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds can also be incorporated into the compositions. The “pharmaceutically acceptable carrier” or “carrier” can further include a pharmaceutically acceptable salt of the compound useful within the invention. Other additional ingredients that can be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

The term “stabilizer,” as used herein, refers to polymers capable of chemically inhibiting or preventing degradation. Stabilizers are added to formulations of compounds to improve chemical and physical stability of the compound.

As used herein, the term “adjuvant” may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as, for example, citric acid, sorbitan monolaurate, triethanolamine oleate, butylated hydroxytoluene, and the like.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “weight percent” is meant to refer to the quantity by weight of a compound and/or component in a composition as the quantity by weight of a constituent component of the composition as a percentage of the weight of the total composition. The weight percent can also be calculated by multiplying the mass fraction by 100. The “mass fraction” is the ratio of one substance of a mass m₁ to the mass of the total composition m_{T} such that weight percent=(m₁/m_{T})*100.

“Aqueous buffer” refers to a water solution which resists change in hydronium ion and the hydroxide ion concentration (and consequent pH) upon addition of small amounts of acid or base, or upon dilution. Buffer solutions consist of a weak acid and its conjugate base (more common) or a weak base and its conjugate acid (less common). The buffer can be prepared by methods well known in the art with the appropriate buffering agents to give the desired pH value. Examples of the suitable buffering agents include hydrochloric acid, lactic acid, acetic acid, citric acid, malic acid, maleic acid, pyruvic acid, succinic acid, tris-hydroxymethylaminomethane, sodium hydroxide, sodium bicarbonate, phosphoric acid, sodium phosphate, and other biologically acceptable buffering agents. Aqueous buffers are readily available commercially and they can be used in preparation of the compositions of this invention without further treatment.

As used herein, the term “hemp extract” refers to a composition of cannabinoids and terpenes that are isolated from a hemp plant. The terms “hemp extract” and “CBD oil” have the same meaning and are used interchangeably herein. The hemp extract can be obtained by any method known in the art. For example, the hemp extract can be obtained by supercritical (or subcritical) CO₂ extraction, which uses carbon dioxide under high pressure and low temperatures to isolate, preserve and maintain the purity of hemp extract. In an embodiment, the hemp extract is obtained from a supercritical CO₂ extraction. For example, supercritical CO₂ extraction may be performed as described in U.S. Pat. No. 8,895,078, which is incorporated herein by reference in its entirety. Alternatively, a solvent such as petroleum ether, ethanol, methanol, butanol, acetone, dry ice, or olive oil can be used, at room temperature (ambient temperature) with stirring, by passive extraction, heated to a temperature above room temperature, or under reflux, as known in the art to provide the hemp extract. In another embodiment, hemp extract from a butanol extraction is employed as starting material for methods disclosed herein.

Suitable methods for measuring the cannabinoid and terpene content in the hemp extract are known in the art. In an embodiment, cannabinoid content is determined using liquid chromatography with mass spectrometry detection (LC-MS). In another embodiment, terpene content is determined using gas chromatography with flame ionization detection (GC-FID) analysis of headspace.

As used herein, the term “flavoring agent” refers to an ingredient that is added to a composition to impart a particular flavor, smell, or other organoleptic property.

As used herein, the term “oil” refers to a nonpolar viscous liquid that is both hydrophobic and lipophilic. Oils may be isolated from animal, vegetable, or petrochemical products.

As used herein, the term “chew” refers to a product or a portion thereof that has rheological and other texture and organoleptic properties which tend to promote chewing upon the article by a target animal. Generally speaking, a chewable matrix will exhibit sufficient ductility that it is at least slightly malleable when bitten by the target animal and sufficient palatability that the target animal is not deterred by its taste from biting it multiple times. By contrast, “chewable” does not mean merely that an article can be chewed by an animal (i.e., it does not mean merely that some portion of the article will fit within an animal's mouth sufficiently to permit engagement of the animal's teeth against the portion).

The “maximal serum concentration level” of a substance, as used herein, refers to the maximal level of the substance found in a plasma sample following a single administration.

As used herein, the term “cold extrusion” refers to a process for producing edible food products comprising several unit operations including mixing, kneading, shearing, shaping, and forming, all of which are conducted at or near ambient temperature.

As used herein, the term “psychotropic effect” refers to a modification of brain function that results in an alteration of perception, mood, consciousness, or behavior.

As used herein, “chemotherapy” is any chemical compound used in the treatment of a proliferative disorder. Examples of chemotherapeutic agents include, without being limited to, the following classes of agents:

nitrogen mustards, e.g. cyclophosphamide, trofosfamide, ifosfamide and chlorambucil;

nitroso ureas, e.g. carmustine (BCNU), lomustine (CCNU), semustine (methyl

CCNU) and nimustine (ACNU);

ethylene imines and methyl-melamines, e.g. thiotepa;

folic acid analogs, e.g. methotrexate;

pyrimidine analogs, e.g. 5-fluorouracil and cytarabine;

purine analogs, e.g. mercaptopurine and azathioprine;

vinca alkaloids, e.g. vinblastine, vincristine and vindesine;

epipodophyllotoxins, e.g. etoposide and teniposide;

antibiotics, e.g. dactinomycin, daunorubicin, doxorubicin, epirubicin, bleomycin a2,

mitomycin c and mitoxantrone;

estrogens, e.g. eiethyl stilbestrol;

gonadotropin-releasing hormone analogs, e.g. leuprolide, buserelin and goserelin;

antiestrogens, e.g. tamoxifen and aminoglutethimide;

androgens, e.g. testolactone and drostanolonproprionate;

platinates, e.g. cisplatin and carboplatin; and

interferons, including interferon-alpha, beta and gamma.

As used herein, the term “quality of life”, or “QoL,” is generally considered a multidimensional concept that involves subjective evaluation of factors that contribute to overall well-being with a more recent publication suggesting a malaise, anxiety and digestive function. Likert scaling system appears to be a sound assessment of QoL (Giuffrida et al. (2018) J Amer Vet Med Assoc. 252:1073-1083.) and is used in this study.

### Pharmaceutical Compositions

In an aspect, provided herein is a pharmaceutical composition comprising hemp extract and a carrier, wherein the hemp extract comprises:

cannabidiol; and

cannabidiolic acid.

In another embodiment, the hemp extract comprises:

cannabidiol;

cannabidiolic acid;

cannabigerolic acid;

Δ9-tetrahydrocannabinol; and

cannabichromene. In another embodiment, the ratio of Δ9-tetrahydrocannabinol to the other cannabinoids is from about 1:50 to about 1:20. In an embodiment, the ratio of cannabidiol to cannabidiolic acid is about 0.1:1 to about 1:0.1. In another embodiment, the ratio of cannabidiol to cannabidiolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabidiol to cannabidiolic acid is about 0.6:1 to about 1:0.6. In still another embodiment, the ratio of cannabidiol to cannabidiolic acid is about 1:1.

In an embodiment, the concentration of Δ9-tetrahydrocannabinol is insufficient to produce a psychotropic effect. In another embodiment, the ratio of Δ9-tetrahydrocannabinol to the other cannabinoids is from about 1:50 to about 1:20. In yet another embodiment, the ratio of Δ9-tetrahydrocannabinol to the other cannabinoids is about 1:50. In still another embodiment, the ratio of Δ9-tetrahydrocannabinol to the other cannabinoids is about 1:45. In an embodiment, the ratio of Δ9-tetrahydrocannabinol to the other cannabinoids is about 1:40. In another embodiment, the ratio of Δ9-tetrahydrocannabinol to the other cannabinoids is about 1:35. In yet another embodiment, the ratio of Δ9-tetrahydrocannabinol to the other cannabinoids is about 1:30. In still another embodiment, the ratio of Δ9-tetrahydrocannabinol to the other cannabinoids is about 1:25. In an embodiment, the ratio of Δ9-tetrahydrocannabinol to the other cannabinoids is about 1:20.

In an embodiment, the concentration of Δ9-tetrahydrocannabinol is less than about 2 mg/mL. In another embodiment, the concentration of Δ9-tetrahydrocannabinol is less than about 1.5 mg/mL. In yet another embodiment, the concentration of Δ9-tetrahydrocannabinol is less than about 1 mg/mL. In still another embodiment, the concentration of Δ9-tetrahydrocannabinol is less than about 0.9 mg/mL. In yet another embodiment, the concentration of Δ9-tetrahydrocannabinol is less than about 0.8 mg/mL. In an embodiment, the concentration of Δ9-tetrahydrocannabinol is less than about 0.7 mg/mL. In another embodiment, the concentration of Δ9-tetrahydrocannabinol is less than about 0.6 mg/mL. In yet another embodiment, the concentration of Δ9-tetrahydrocannabinol is less than about 0.5 mg/mL. In still another embodiment, the concentration of Δ9-tetrahydrocannabinol is less than about 0.4 mg/mL. In an embodiment, the concentration of Δ9-tetrahydrocannabinol is less than about 0.3 mg/mL. In another embodiment, the concentration of Δ9-tetrahydrocannabinol is less than about 0.2 mg/mL. In yet another embodiment, the concentration of Δ9-tetrahydrocannabinol is less than about 0.1 mg/mL. In another embodiment, the concentration of Δ9-tetrahydrocannabinol is about 0 mg/mL.

In an embodiment, the hemp extract comprises:

about 0.1-20 mg/mL of cannabidiol;

about 0.1-20 mg/mL of cannabidiolic acid;

about 0.01-0.5 mg/mL cannabigerolic acid;

about 0.01-0.5 mg/mL Δ9-tetrahydrocannabinol; and

about 0.01-0.5 mg/mL cannabichromene.

In another embodiment, the hemp extract comprises:

about 1-10 mg/mL of cannabidiol;

about 1-10 mg/mL of cannabidiolic acid;

about 0.05-0.2 mg/mL cannabigerolic acid;

about 0.1-0.3 mg/mL Δ9-tetrahydrocannabinol; and

about 0.1-0.4 mg/mL cannabichromene.

In yet another embodiment, the hemp extract comprises:

about 5 mg/mL of cannabidiol;

about 5 mg/mL of cannabidiolic acid;

about 0.11 mg/mL cannabigerolic acid;

about 0.25 mg/mL Δ9-tetrahydrocannabinol; and

about 0.27 mg/mL cannabichromene.

In an embodiment, provided herein is a pharmaceutical composition comprising hemp extract and a carrier, wherein the hemp extract comprises:

α-pinene;

β-myrcene;

β-pinene;

δ-limonene;

linalool;

β-caryophyllene;

α-humulene;

nerolidol 2;

guaiol;

caryophyllene oxide; and

α-bisabolol.

In another embodiment, the hemp extract comprises:

about 0.09-0.13% α-pinene;

about 0.23-0.44% β-myrcene;

about 0.04-0.09% β-pinene;

about 0.05-0.09% δ-limonene;

about 0.03-0.06% linalool;

about 0.04-0.07% β-caryophyllene;

about 0.02-0.04% α-humulene;

about 0.04-0.07% nerolidol 2;

about 0.02-0.04% guaiol;

about 0.04-0.08% caryophyllene oxide; and

about 0.01-0.04% α-bisabolol.

In another embodiment, the hemp extract comprises:

about 0.07-0.30% α-pinene;

about 0.10-0.60% β-myrcene;

about 0.02-0.20% β-pinene;

about 0.03-0.20% δ-limonene;

about 0.01-0.08% linalool;

about 0.03-0.09% β-caryophyllene;

about 0.01-0.06% α-humulene;

about 0.02-0.09% nerolidol 2; and

about 0.01-0.06% guaiol;

In another embodiment, the hemp extract comprises:

about 0.01-0.50% α-pinene;

about 0.01-0.90% β-myrcene;

about 0.01-0.50% β-pinene;

about 0.01-0.50% δ-limonene;

about 0.01-0.50% linalool;

about 0.01-0.50% β-caryophyllene;

about 0.01-0.50% α-humulene;

about 0.01-0.50% nerolidol 2;

about 0.01-0.50% guaiol;

about 0.01-0.50% caryophyllene oxide; and

about 0.01-0.50% α-bisabolol.

In another embodiment, the hemp extract further comprises:

camphene;

β-ocimene;

eucalyptol;

isopulegol; and/or

nerolidol 1.

In another embodiment, the hemp extract comprises:

about 0.02% camphene;

about 0.02-0.03% β-ocimene;

about 0.02-0.05% eucalyptol;

about 0.02% isopulegol; and/or

about 0.02-0.04% nerolidol 1.

In another embodiment, the hemp extract comprises:

about 0.01-0.04% camphene;

about 0.01-0.05% β-ocimene;

about 0.01-0.07% eucalyptol;

about 0.01-0.04% isopulegol; and/or

about 0.01-0.05% nerolidol 1.

In another embodiment, the hemp extract comprises:

about 0.01-0.50% camphene;

about 0.01-0.50% β-ocimene;

about 0.01-0.50% eucalyptol;

about 0.01-0.50% isopulegol; and/or

about 0.01-0.50% nerolidol 1.

In an embodiment, the hemp extract does not comprise terpenes.

In an embodiment, the hemp extract comprises 1 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 2 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 3 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 4 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 5 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 6 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 7 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 8 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 9 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 10 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 11 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 12 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 13 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 14 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 15 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the composition is formulated as an oil. In another embodiment, the carrier is selected from the group consisting of linseed oil, olive oil, fish oil, salmon oil, coconut oil, catnip oil, sesame oil, MCT oil, and grapeseed oil. In yet another embodiment, the carrier is grapeseed oil.

In an embodiment, the dosage form comprises nepetalactone.

In an embodiment, the dosage form comprises taurine.

In an embodiment, the pharmaceutical composition comprises lecithin. In another embodiment, the lecithin is sunflower lecithin. In another embodiment, the lecithin is about 5%, about 10%, about 15%, about 20%, about 25%, about 30%, about 35%, about 40%, about 45%, or 50% w/v of the pharmaceutical composition. In other embodiments, the pharmaceutical composition comprises lecithin mixed with another oil. In some embodiments, the other oil is sesame oil. In some embodiments, the lecithin and other oil are mixed at a ratio of about 1:4, 1:2, 1:1, 2:1 or 4:1. In some embodiments, the pharmaceutical composition comprises hemp extract and a carrier oil. In some embodiments, the carrier oil is substantially all lecithin. In other embodiments, the carrier oil is 100% lecithin.

In an embodiment, the pharmaceutical composition comprises NF-971P. In an embodiment, the NF-971P is about 0.5%, about 1.0%, about 1.5%, about 2.0%, about 2.5%, or about 3.0% weight/volume ratio of the pharmaceutical composition.

In an embodiment, the pharmaceutical composition is formulated as a sublingual spray. In still another embodiment, the pharmaceutical composition is formulated as a water or alcohol soluble solution, a gel, or a cream for transdermal application. In an embodiment, the pharmaceutical composition is formulated as a gel for buccal or mucosal administration. In an embodiment, the pharmaceutical composition is formulated as a powder. In another embodiment, the pharmaceutical composition is formulated as a solution for subcutaneous injection. In yet another embodiment, the pharmaceutical composition is formulated as a tablet. In still another embodiment, the pharmaceutical composition is formulated as a capsule. In an embodiment, the pharmaceutical composition is formulated as a hard chewable. In an embodiment, the pharmaceutical composition is formulated as a soft chewable.

In an embodiment, the composition is formulated as a chew for oral administration. In another embodiment, the chew is produced using cold extrusion. In another embodiment, the weight of the chew is about 0.5-10 g. In yet another embodiment, the weight of the chew is about 4 g, about 6 g, about 9 g, or about 10 g. In still another embodiment, the weight of the chew is about 0.5 g. In an embodiment, the weight of the chew is about 1 g. In another embodiment, the weight of the chew is about 1.5 g. In yet another embodiment, the weight of the chew is about 2 g. In still another embodiment, the weight of the chew is about 3 g. In an embodiment, the weight of the chew is about 4 g. In another embodiment, the weight of the chew is about 5 g. In yet another embodiment, the weight of the chew is about 6 g. In still another embodiment, the weight of the chew is about 7 g. In an embodiment, the weight of the chew is about 8 g. In another embodiment, the weight of the chew is about 9 g. In yet another embodiment, the weight of the chew is about 10 g.

In an embodiment, the 4 g chew comprises:

about 7 mg of cannabidiol;

about 6 mg of cannabidiolic acid;

about 0.12 mg cannabigerolic acid;

about 0.32 mg Δ9-tetrahydrocannabinol; and

about 0.36 mg cannabichromene.

The pharmaceutical compositions of the present disclosure may be manufactured by processes well known in the art, e.g., by means of conventional mixing, dissolving, granulating, grinding, pulverizing, dragee-making, levigating, emulsifying, encapsulating, entrapping or by lyophilizing processes.

The compositions for use in accordance with the present disclosure thus may be formulated in conventional manner using one or more pharmaceutically acceptable carriers comprising excipients and auxiliaries, which facilitate processing of the active compounds into preparations which can be used pharmaceutically. Proper formulation is dependent upon the route of administration chosen.

### Dosage Forms

In an aspect, provided herein is a dosage form comprising:

cannabidiol; and

cannabidiolic acid; and

one or more pharmaceutically acceptable additives, flavoring agents, surfactants, and adjuvants.

In another embodiment, the dosage form comprises:

cannabidiol;

cannabidiolic acid;

cannabigerolic acid;

Δ9-tetrahydrocannabinol;

cannabichromene; and

one or more pharmaceutically acceptable additives, flavoring agents, surfactants, and adjuvants.

In an embodiment, the ratio of cannabidiol to cannabidiolic acid is selected from the group consisting of about 1:100, about 1:50, about 1:10, and about 1:1. In an embodiment, the ratio of cannabidiol to cannabidiolic acid is about 0.1:1 to about 1:0.1. In another embodiment, the ratio of cannabidiol to cannabidiolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabidiol to cannabidiolic acid is about 0.6:1 to about 1:0.6. In still another embodiment, the ratio of cannabidiol to cannabidiolic acid is about 1:1.

In an embodiment, the concentration of Δ9-tetrahydrocannabinol is insufficient to produce a psychotropic effect. In another embodiment, the ratio of Δ9-tetrahydrocannabinol to the other cannabinoids is from about 1:50 to about 1:20. In yet another embodiment, the ratio of Δ9-tetrahydrocannabinol to the other cannabinoids is about 1:50. In still another embodiment, the ratio of Δ9-tetrahydrocannabinol to the other cannabinoids is about 1:45. In an embodiment, the ratio of Δ9-tetrahydrocannabinol to the other cannabinoids is about 1:40. In another embodiment, the ratio of Δ9-tetrahydrocannabinol to the other cannabinoids is about 1:35. In yet another embodiment, the ratio of Δ9-tetrahydrocannabinol to the other cannabinoids is about 1:30. In still another embodiment, the ratio of Δ9-tetrahydrocannabinol to the other cannabinoids is about 1:25. In an embodiment, the ratio of Δ9-tetrahydrocannabinol to the other cannabinoids is about 1:20.

In an embodiment, the concentration of Δ9-tetrahydrocannabinol is less than about 2 mg/mL. In another embodiment, the concentration of Δ9-tetrahydrocannabinol is less than about 1.5 mg/mL. In yet another embodiment, the concentration of Δ9-tetrahydrocannabinol is less than about 1 mg/mL. In still another embodiment, the concentration of Δ9-tetrahydrocannabinol is less than about 0.9 mg/mL. In yet another embodiment, the concentration of Δ9-tetrahydrocannabinol is less than about 0.8 mg/mL. In an embodiment, the concentration of Δ9-tetrahydrocannabinol is less than about 0.7 mg/mL. In another embodiment, the concentration of Δ9-tetrahydrocannabinol is less than about 0.6 mg/mL. In yet another embodiment, the concentration of Δ9-tetrahydrocannabinol is less than about 0.5 mg/mL. In still another embodiment, the concentration of Δ9-tetrahydrocannabinol is less than about 0.4 mg/mL. In an embodiment, the concentration of Δ9-tetrahydrocannabinol is less than about 0.3 mg/mL. In another embodiment, the concentration of Δ9-tetrahydrocannabinol is less than about 0.2 mg/mL. In yet another embodiment, the concentration of Δ9-tetrahydrocannabinol is less than about 0.1 mg/mL. In another embodiment, the concentration of Δ9-tetrahydrocannabinol is about 0 mg/mL.

In an embodiment, the dosage form comprises:

about 0.1-20 mg/mL of cannabidiol;

about 0.1-20 mg/mL of cannabidiolic acid;

about 0.01-0.5 mg/mL cannabigerolic acid;

about 0.01-0.5 mg/mL Δ9-tetrahydrocannabinol; and

about 0.01-0.5 mg/mL cannabichromene.

In another embodiment, the dosage form comprises:

about 1-10 mg/mL of cannabidiol;

about 1-10 mg/mL of cannabidiolic acid;

about 0.05-0.2 mg/mL cannabigerolic acid;

about 0.1-0.3 mg/mL Δ9-tetrahydrocannabinol; and

about 0.1-0.4 mg/mL cannabichromene.

In yet another embodiment, the dosage form comprises:

about 5 mg/mL of cannabidiol;

about 5 mg/mL of cannabidiolic acid;

about 0.11 mg/mL cannabigerolic acid;

about 0.25 mg/mL Δ9-tetrahydrocannabinol; and

about 0.27 mg/mL cannabichromene.

In some embodiments, the dosage form comprises:

α-pinene;

β-myrcene;

β-pinene;

δ-limonene;

linalool;

β-caryophyllene;

α-humulene;

nerolidol 2;

guaiol;

caryophyllene oxide; and α-bisabolol.

In another embodiment, the dosage form comprises:

about 0.09-0.13% α-pinene;

about 0.23-0.44% β-myrcene;

about 0.04-0.09% β-pinene;

about 0.05-0.09% δ-limonene;

about 0.03-0.06% linalool;

about 0.04-0.07% β-caryophyllene;

about 0.02-0.04% α-humulene;

about 0.04-0.07% nerolidol 2;

about 0.02-0.04% guaiol;

about 0.04-0.08% caryophyllene oxide; and

about 0.01-0.04% α-bisabolol.

In another embodiment, the dosage form comprises:

about 0.07-0.30% α-pinene;

about 0.10-0.60% β-myrcene;

about 0.02-0.20% β-pinene;

about 0.03-0.20% δ-limonene;

about 0.01-0.08% linalool;

about 0.03-0.09% β-caryophyllene;

about 0.01-0.06% α-humulene;

about 0.02-0.09% nerolidol 2; and

about 0.01-0.06% guaiol;

In another embodiment, the dosage form comprises:

about 0.01-0.50% α-pinene;

about 0.01-0.90% β-myrcene;

about 0.01-0.50% β-pinene;

about 0.01-0.50% δ-limonene;

about 0.01-0.50% linalool;

about 0.01-0.50% β-caryophyllene;

about 0.01-0.50% α-humulene;

about 0.01-0.50% nerolidol 2;

about 0.01-0.50% guaiol;

about 0.01-0.50% caryophyllene oxide; and

about 0.01-0.50% α-bisabolol.

In another embodiment, the dosage form further comprises:

camphene;

β-ocimene;

eucalyptol;

isopulegol; and/or

nerolidol 1.

In another embodiment, the dosage form comprises:

about 0.02% camphene;

about 0.02-0.03% β-ocimene;

about 0.02-0.05% eucalyptol;

about 0.02% isopulegol; and/or

about 0.02-0.04% nerolidol 1.

In another embodiment, the dosage form comprises:

about 0.01-0.04% camphene;

about 0.01-0.05% β-ocimene;

about 0.01-0.07% eucalyptol;

about 0.01-0.04% isopulegol; and/or

about 0.01-0.05% nerolidol 1.

In another embodiment, the dosage form comprises:

about 0.01-0.50% camphene;

about 0.01-0.50% β-ocimene;

about 0.01-0.50% eucalyptol;

about 0.01-0.50% isopulegol; and/or

about 0.01-0.50% nerolidol 1.

In an embodiment, the hemp extract does not comprise terpenes.

In an embodiment, the hemp extract comprises 1 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 2 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 3 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 4 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the dosage form comprises 5 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the dosage form comprises 6 or more of the following: α-pinene, β-myrcene, β-pinene, δ-limonene, linalool, β-caryophyIlene, α-humulene, nerolidol 2, guaiol, caryophyllene oxide, α-bisabolol, camphene, β-ocimene, eucalyptol, isopulegol, and nerolidol 1.

In an embodiment, the dosage form comprises 7 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 8 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 9 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 10 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 11 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 12 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 13 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 14 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 15 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1

In an embodiment, the flavoring agent is selected from the group consisting of catnip oil, peppermint oil, mango extract, beef, poultry, and seafood.

In an embodiment, the dosage form is formulated as a sublingual spray. In still another embodiment, the dosage form is formulated as a water or alcohol soluble solution, a gel, or a cream for transdermal application. In an embodiment, the dosage form is formulated as a powder. In an embodiment, the dosage form is formulated as a gel for buccal or mucosal administration. In another embodiment, the dosage form is formulated as a solution for subcutaneous injection. In yet another embodiment, the dosage form is formulated as a tablet. In still another embodiment, the dosage form is formulated as a capsule. In an embodiment, the dosage form is formulated as a hard chewable. In an embodiment, the dosage form is formulated as a soft chewable.

In some embodiments, the invention includes infusing edible products with hemp extract. In another embodiment, the edible product is an extruded food product, baked food product, nut butter, spread, pelleted feed, or processed food. In another embodiment, the edible product is a pet food. In another embodiment the pet food is in a dry, shelf-stable form such as dried meals, dried fish, dried dairy products, fish meal, fish flour, cereals, flours, carbohydrates, dried fruits, etc. In another embodiment, the pet food is moist or semi-moist. In another embodiment, the pet food contains food additives or supplements such as vitamins, minerals, medicinals, etc., for example chemicals, enzymes, etc., capable of removing plaque or tartar from the animal's teeth, etc.

In an embodiment, the hemp extract is administered with catnip oil. In another embodiment, any of the dosage forms described can also include catnip.

In another embodiment, hemp extracts are administered using a nebulizer. In another embodiment, the nebulizer delivery device and system is capable of effectively and efficiently administering one or more nebulized drug to an animal. In another embodiment, the nebulizer system can easily be used on animals without removing them from their natural environment. In another embodiment, the nebulizer delivery device and system enables animals to be easily treated daily or multiple times a day without undue stress or the need for extensive resources. In another embodiment, the nebulizer delivery device and system can be used on animals having varying levels of training.

In one embodiment, hemp extract is administered using a diffuser. The diffuser can be any device which disperses hemp extract into the air. Hemp extract may be dispersed by any method, including by natural convection, by forced convection, by heating a wick or pad, for example, holding the hemp extract, by using pumps, or with fans.

In one embodiment, hemp extract is administered by a pet collar. The pet collar may comprise a belt with a buckle on one side, a free end on the other side and an attachment means, such as apertures disposed longitudinally within the central portion of the belt, or a quick release clasp mechanism, for securing the collar in a closed loop configuration. The pet collar may be made from a variety of materials including nylon, polyester leather or other suitable material. The belt material may be treated with a water-proofing compound. The nylon or polyester belt may be interwoven with reflective fibers to enhance the visibility of the pet collar during nighttime hours. In one embodiment, the collar is infused with hemp extract.

### Chews

In an embodiment, the dosage form is formulated as a chew for oral administration. In another embodiment, the chew is produced using cold extrusion. In another embodiment, the weight of the chew is about 0.5-10 g. In yet another embodiment, the weight of the chew is about 4 g, about 6 g, about 9 g, or about 10 g. In still another embodiment, the weight of the chew is about 0.5 g. In an embodiment, the weight of the chew is about 1 g. In another embodiment, the weight of the chew is about 1.5 g. In yet another embodiment, the weight of the chew is about 2 g. In still another embodiment, the weight of the chew is about 3 g. In an embodiment, the weight of the chew is about 4 g. In another embodiment, the weight of the chew is about 5 g. In yet another embodiment, the weight of the chew is about 6 g. In still another embodiment, the weight of the chew is about 7 g. In an embodiment, the weight of the chew is about 8 g. In another embodiment, the weight of the chew is about 9 g. In yet another embodiment, the weight of the chew is about 10 g.

In one embodiment, the dosage form comprises:

brewer's yeast;

arabic gum;

guar gum;

a flavoring agent;

Verdilox;

Previon;

hemp extract;

glycerin;

sunflower lecithin; and

water.

In another embodiment, the dosage form comprises:

about 25-35% brewer's yeast;

about 1-10% arabic gum;

about 0.1-4% guar gum;

about 10-20% of a flavoring agent;

about 0.01-1% Verdilox;

about 0.1-2% Previon;

about 1-10% hemp extract;

about 10-20% glycerin;

about 1-10% sunflower lecithin; and

about 1-10% water.

In another embodiment, the dosage form comprises:

about 29-33% brewer's yeast;

about 3-6% arabic gum;

about 0.5-2% guar gum;

about 12-16% of a flavoring agent;

about 0.01-0.1% Verdilox;

about 0.5-1.5% Previon;

about 3-6% hemp extract;

about 13-17% glycerin;

about 3-7% sunflower lecithin; and

about 3-7% water.

In yet another embodiment, the dosage form comprises:

about 30% brewer's yeast;

about 4.7% arabic gum;

about 0.9% guar gum;

about 14.2% of a flavoring agent;

about 0.05% Verdilox;

about 0.9% Previon;

about 4.7% hemp extract;

about 15.1% glycerin;

about 5.7% sunflower lecithin; and

about 5.7% water.

In one embodiment, the dosage form comprises:

glucosamine HCl;

brewer's yeast;

arabic gum;

guar gum;

a flavoring agent;

Verdilox;

Previon;

hemp extract;

glycerin;

sunflower lecithin; and

water.

In another embodiment, the dosage form comprises:

about 10-20% glucosamine HCl;

about 25-35% brewer's yeast;

about 1-10% arabic gum;

about 0.1-4% guar gum;

about 10-20% of a flavoring agent;

about 0.01-1% Verdilox;

about 0.1-2% Previon;

about 1-10% hemp extract;

about 10-20% glycerin;

about 1-10% sunflower lecithin; and

about 1-10% water.

In another embodiment, the dosage form comprises:

about 12-17% glucosamine HCl;

about 29-33% brewer's yeast;

about 3-6% arabic gum;

about 0.5-2% guar gum;

about 12-16% of a flavoring agent;

about 0.01-0.1% Verdilox;

about 0.5-1.5% Previon;

about 3-6% hemp extract;

about 13-17% glycerin;

about 3-7% sunflower lecithin; and

about 3-7% water.

In yet another embodiment, the dosage form comprises:

about 15.6% glucosamine HCl;

about 30% brewer's yeast;

about 4.7% arabic gum;

about 0.9% guar gum;

about 14.2% of a flavoring agent;

about 0.05% Verdilox;

about 0.9% Previon;

about 4.7% hemp extract;

about 15.1% glycerin;

about 5.7% sunflower lecithin; and

about 5.7% water.

In one embodiment, the dosage form comprises:

glucosamine HCl;

chondroitin sulfate (76%);

brewer's yeast;

arabic gum;

guar gum;

a flavoring agent;

Verdilox;

Previon;

hemp extract;

glycerin;

sunflower lecithin; and

water.

In another embodiment, the dosage form comprises:

about 10-20% glucosamine HCl;

about 0.1-7% chondroitin sulfate (76%);

about 25-35% brewer's yeast;

about 1-10% arabic gum;

about 0.1-4% guar gum;

about 10-20% of a flavoring agent;

about 0.01-1% Verdilox;

about 0.1-2% Previon;

about 1-10% hemp extract;

about 10-20% glycerin;

about 1-10% sunflower lecithin; and

about 1-10% water.

In another embodiment, the dosage form comprises:

about 12-17% glucosamine HCl;

about 1-4% chondroitin sulfate (76%);

about 29-33% brewer's yeast;

about 3-6% arabic gum;

about 0.5-2% guar gum;

about 12-16% of a flavoring agent;

about 0.01-0.1% Verdilox;

about 0.5-1.5% Previon;

about 3-6% hemp extract;

about 13-17% glycerin;

about 3-7% sunflower lecithin; and

about 3-7% water.

In yet another embodiment, the dosage form comprises:

about 15.6% glucosamine HCl;

about 2.6% chondroitin sulfate (76%);

about 30% brewer's yeast;

about 4.7% arabic gum;

about 0.9% guar gum;

about 14.2% of a flavoring agent;

about 0.05% Verdilox;

about 0.9% Previon;

about 4.7% hemp extract;

about 15.1% glycerin;

about 5.7% sunflower lecithin; and

about 5.7% water.

In another embodiment, the dosage form comprises:

hyaluronic acid;

brewer's yeast;

arabic gum;

guar gum;

a flavoring agent;

Verdilox;

Previon;

hemp extract;

glycerin;

sunflower lecithin; and

water.

In another embodiment, the dosage form comprises:

about 0.01-3% hyaluronic acid;

about 25-35% brewer's yeast;

about 1-10% arabic gum;

about 0.1-5% guar gum;

about 10-20% of a flavoring agent;

about 0.01-1% Verdilox;

about 0.1-3% Previon;

about 1-10% hemp extract;

about 10-20% glycerin;

about 1-10% sunflower lecithin; and

about 1-10% water.

In another embodiment, the dosage form comprises:

about 0.01-1% hyaluronic acid;

about 29-33% brewer's yeast;

about 3-6% arabic gum;

about 0.5-2% guar gum;

about 12-16% of a flavoring agent;

about 0.01-0.1% Verdilox;

about 0.5-1.5% Previon;

about 3-6% hemp extract;

about 13-17% glycerin;

about 3-7% sunflower lecithin; and

about 3-7% water.

In yet another embodiment, the dosage form comprises:

about 0.1% hyaluronic acid;

about 30.6% brewer's yeast;

about 4.8% arabic gum;

about 0.97% guar gum;

about 14.5% of a flavoring agent;

about 0.05% Verdilox;

about 0.97% Previon;

about 4.8% hemp extract;

about 15.5% glycerin;

about 5.8% sunflower lecithin; and

about 5.8% water.

In another embodiment, the dosage form comprises:

glucosamine HCl;

hyaluronic acid;

brewer's yeast;

arabic gum;

guar gum;

a flavoring agent;

Verdilox;

Previon;

hemp extract;

glycerin;

sunflower lecithin; and

water.

In another embodiment, the dosage form comprises:

about 10-20% glucosamine HCl;

about 0.01-3% hyaluronic acid;

about 25-35% brewer's yeast;

about 1-10% arabic gum;

about 0.1-5% guar gum;

about 10-20% of a flavoring agent;

about 0.01-1% Verdilox;

about 0.1-3% Previon;

about 1-10% hemp extract;

about 10-20% glycerin;

about 1-10% sunflower lecithin; and

about 1-10% water.

In another embodiment, the dosage form comprises:

about 12-17% glucosamine HCl;

about 0.01-1% hyaluronic acid;

about 29-33% brewer's yeast;

about 3-6% arabic gum;

about 0.5-2% guar gum;

about 12-16% of a flavoring agent;

about 0.01-0.1% Verdilox;

about 0.5-1.5% Previon;

about 3-6% hemp extract;

about 13-17% glycerin;

about 3-7% sunflower lecithin; and

about 3-7% water.

In yet another embodiment, the dosage form comprises:

about 16% glucosamine HCl;

about 0.1% hyaluronic acid;

about 30.6% brewer's yeast;

about 4.8% arabic gum;

about 0.97% guar gum;

about 14.5% of a flavoring agent;

about 0.05% Verdilox;

about 0.97% Previon;

about 4.8% hemp extract;

about 15.5% glycerin;

about 5.8% sunflower lecithin; and

about 5.8% water.

In yet another embodiment, the dosage form comprises:

hemp extract;

peanut butter;

rice bran;

glucosamine HCL;

sweet potato;

dry molasses;

sorbic acid

brewer's yeast;

sugar;

water;

glycerin;

potato starch;

dehydrated peanut butter;

rice starch; and

guar gum.

In yet another embodiment, the dosage form comprises:

about 5.0% hemp extract;

about 15.0% peanut butter;

about 12.5% rice bran;

about 5.5% sweet potato;

about 8.0% dry molasses;

about 1% sorbic acid;

about 5.0% brewer's yeast;

about 6.0% sugar;

about 9.25% water;

about 13.0 glycerin;

about 2.0% potato starch;

about 1.0% dehydrated peanut butter;

about 2.0% rice starch; and

about 2.0% guar gum.

In yet another embodiment, the dosage form comprises:

about 5.0% hemp extract;

about 15.0% peanut butter;

about 13.0% rice bran;

about 6.0% sweet potato;

about 9.0% dry molasses;

about 1% sorbic acid;

about 5.0% brewer's yeast;

about 6.0% sugar;

about 9.5% water;

about 13.0 glycerin;

about 4.0% potato starch;

about 1.0% dehydrated peanut butter;

about 2.0% rice starch; and

about 2.0% guar gum.

In yet another embodiment, the dosage form comprises:

about 3.0-10.0% hemp extract;

about 10.0-20.0% peanut butter;

about 10.0-15.0% rice bran;

about 4.0-10.0% sweet potato;

about 6.0-13.0% dry molasses;

about 0.5-5.0% sorbic acid;

about 2.0-8.0% brewer's yeast;

about 3.0-8.0% sugar;

about 5.0-15.0% water;

about 8.0-18.0% glycerin;

about 1.0-8.0% potato starch;

about 0.5-5.0% dehydrated peanut butter;

about 1.0-5.0% rice starch; and

about 1.0-5.0% guar gum.

In yet another embodiment, the dosage form comprises:

about 5.0% hemp extract;

about 15.0% peanut butter;

about 12.5% rice bran;

about 12.75% glucosamine HCL;

about 5.5% sweet potato;

about 8.0% dry molasses;

about 1% sorbic acid;

about 5.0% brewer's yeast;

about 6.0% sugar;

about 9.25% water;

about 13.0 glycerin;

about 2.0% potato starch;

about 1.0% dehydrated peanut butter;

about 2.0% rice starch; and

about 2.0% guar gum.

In yet another embodiment, the dosage form comprises:

about 5.0% hemp extract;

about 15.0% peanut butter;

about 13.0% rice bran;

about 8.5% glucosamine HCL;

about 6.0% sweet potato;

about 9.0% dry molasses;

about 1% sorbic acid;

about 5.0% brewer's yeast;

about 6.0% sugar;

about 9.5% water;

about 13.0 glycerin;

about 4.0% potato starch;

about 1.0% dehydrated peanut butter;

about 2.0% rice starch; and

about 2.0% guar gum.

In yet another embodiment, the dosage form comprises:

about 3.0-10.0% hemp extract;

about 10.0-20.0% peanut butter;

about 10.0-15.0% rice bran;

about 5.0-15.0% glucosamine HCL;

about 4.0-10.0% sweet potato;

about 6.0-13.0% dry molasses;

about 0.5-5.0% sorbic acid;

about 2.0-8.0% brewer's yeast;

about 3.0-8.0% sugar;

about 5.0-15.0% water;

about 8.0-18.0% glycerin;

about 1.0-8.0% potato starch;

about 0.5-5.0% dehydrated peanut butter;

about 1.0-5.0% rice starch; and

about 1.0-5.0% guar gum.

In another embodiment, the dosage form further comprises chondroitin sulfate.

In another embodiment, the dosage form comprises 2.0% hemp extract. In another embodiment, the dosage form comprises 3.0% hemp extract. In another embodiment, the dosage form comprises 4.0% hemp extract. In another embodiment, the dosage form comprises 5.0% hemp extract. In another embodiment, the dosage form comprises 6.0% hemp extract. In another embodiment, the dosage form comprises 7.0% hemp extract. In another embodiment, the dosage form comprises 8.0% hemp extract. In another embodiment, the dosage form comprises 9.0% hemp extract. In another embodiment, the dosage form comprises 10.0% hemp extract.

In an embodiment, the hemp extract comprises:

cannabidiol;

cannabidiolic acid;

cannabigerolic acid;

Δ9-tetrahydrocannabinol; and

cannabichromene.

In an embodiment, the ratio of cannabidiol to cannabidiolic acid is selected from the group consisting of about 1:100, about 1:50, about 1:10, and about 1:1. In an embodiment, the ratio of cannabidiol to cannabidiolic acid is about 0.1:1 to about 1:0.1. In another embodiment, the ratio of cannabidiol to cannabidiolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabidiol to cannabidiolic acid is about 0.6:1 to about 1:0.6. In still another embodiment, the ratio of cannabidiol to cannabidiolic acid is about 1:1.

In an embodiment, the concentration of Δ9-tetrahydrocannabinol is insufficient to produce a psychotropic effect. In another embodiment, the ratio of Δ9-tetrahydrocannabinol to the other cannabinoids is from about 1:50 to about 1:20. In yet another embodiment, the ratio of Δ9-tetrahydrocannabinol to the other cannabinoids is about 1:50. In still another embodiment, the ratio of Δ9-tetrahydrocannabinol to the other cannabinoids is about 1:45. In an embodiment, the ratio of Δ9-tetrahydrocannabinol to the other cannabinoids is about 1:40. In another embodiment, the ratio of Δ9-tetrahydrocannabinol to the other cannabinoids is about 1:35. In yet another embodiment, the ratio of Δ9-tetrahydrocannabinol to the other cannabinoids is about 1:30. In still another embodiment, the ratio of Δ9-tetrahydrocannabinol to the other cannabinoids is about 1:25. In an embodiment, the ratio of Δ9-tetrahydrocannabinol to the other cannabinoids is about 1:20.

In an embodiment, the concentration of Δ9-tetrahydrocannabinol is less than about 2 mg/mL. In another embodiment, the concentration of Δ9-tetrahydrocannabinol is less than about 1.5 mg/mL. In yet another embodiment, the concentration of Δ9-tetrahydrocannabinol is less than about 1 mg/mL. In still another embodiment, the concentration of Δ9-tetrahydrocannabinol is less than about 0.9 mg/mL. In yet another embodiment, the concentration of Δ9-tetrahydrocannabinol is less than about 0.8 mg/mL. In an embodiment, the concentration of Δ9-tetrahydrocannabinol is less than about 0.7 mg/mL. In another embodiment, the concentration of Δ9-tetrahydrocannabinol is less than about 0.6 mg/mL. In yet another embodiment, the concentration of Δ9-tetrahydrocannabinol is less than about 0.5 mg/mL. In still another embodiment, the concentration of Δ9-tetrahydrocannabinol is less than about 0.4 mg/mL. In an embodiment, the concentration of Δ9-tetrahydrocannabinol is less than about 0.3 mg/mL. In another embodiment, the concentration of Δ9-tetrahydrocannabinol is less than about 0.2 mg/mL. In yet another embodiment, the concentration of Δ9-tetrahydrocannabinol is less than about 0.1 mg/mL. In yet another embodiment, the concentration of Δ9-tetrahydrocannabinol is about 0 mg/mL.

In an embodiment, the hemp extract comprises:

about 0.1-20 mg/mL of cannabidiol;

about 0.1-20 mg/mL of cannabidiolic acid;

about 0.01-0.5 mg/mL cannabigerolic acid;

about 0.01-0.5 mg/mL Δ9-tetrahydrocannabinol; and

about 0.01-0.5 mg/mL cannabichromene.

In another embodiment, the hemp extract comprises:

about 1-10 mg/mL of cannabidiol;

about 1-10 mg/mL of cannabidiolic acid;

about 0.05-0.2 mg/mL cannabigerolic acid;

about 0.1-0.3 mg/mL Δ9-tetrahydrocannabinol; and

about 0.1-0.4 mg/mL cannabichromene.

In yet another embodiment, the hemp extract comprises:

about 5 mg/mL of cannabidiol;

about 5 mg/mL of cannabidiolic acid;

about 0.11 mg/mL cannabigerolic acid;

about 0.25 mg/mL Δ9-tetrahydrocannabinol; and

about 0.27 mg/mL cannabichromene.

In an embodiment, the hemp extract comprises:

α-pinene;

β-myrcene;

β-pinene;

δ-limonene;

linalool;

β-caryophyllene;

α-humulene;

nerolidol 2;

guaiol;

caryophyllene oxide; and

α-bisabolol.

In another embodiment, the hemp extract comprises:

about 0.09-0.13% α-pinene;

about 0.23-0.44% β-myrcene;

about 0.04-0.09% β-pinene;

about 0.05-0.09% δ-limonene;

about 0.03-0.06% linalool;

about 0.04-0.07% β-caryophyllene;

about 0.02-0.04% α-humulene;

about 0.04-0.07% nerolidol 2;

about 0.02-0.04% guaiol;

about 0.04-0.08% caryophyllene oxide; and

about 0.01-0.04% α-bisabolol.

In another embodiment, the hemp extract comprises:

about 0.07-0.30% α-pinene;

about 0.10-0.60% β-myrcene;

about 0.02-0.20% β-pinene;

about 0.03-0.20% δ-limonene;

about 0.01-0.08% linalool;

about 0.03-0.09% β-caryophyllene;

about 0.01-0.06% α-humulene;

about 0.02-0.09% nerolidol 2; and

about 0.01-0.06% guaiol;

In another embodiment, the hemp extract comprises:

about 0.01-0.50% α-pinene;

about 0.01-0.90% β-myrcene;

about 0.01-0.50% β-pinene;

about 0.01-0.50% δ-limonene;

about 0.01-0.50% linalool;

about 0.01-0.50% β-caryophyllene;

about 0.01-0.50% α-humulene;

about 0.01-0.50% nerolidol 2;

about 0.01-0.50% guaiol;

about 0.01-0.50% caryophyllene oxide; and

about 0.01-0.50% α-bisabolol.

In another embodiment, the hemp extract further comprises:

camphene;

β-ocimene;

eucalyptol;

isopulegol; and/or

nerolidol 1.

In another embodiment, the hemp extract comprises:

about 0.02% camphene;

about 0.02-0.03% β-ocimene;

about 0.02-0.05% eucalyptol;

about 0.02% isopulegol; and/or

about 0.02-0.04% nerolidol 1.

In another embodiment, the hemp extract comprises:

about 0.01-0.04% camphene;

about 0.01-0.05% β-ocimene;

about 0.01-0.07% eucalyptol;

about 0.01-0.04% isopulegol; and/or

about 0.01-0.05% nerolidol 1.

In another embodiment, the hemp extract comprises:

about 0.01-0.50% camphene;

about 0.01-0.50% β-ocimene;

about 0.01-0.50% eucalyptol;

about 0.01-0.50% isopulegol; and/or

about 0.01-0.50% nerolidol 1.

In an embodiment, the composition is formulated as an oil. In another embodiment, the carrier is selected from the group consisting of linseed oil, olive oil, fish oil, salmon oil, coconut oil, catnip oil, sesame oil, MCT oil, and grapeseed oil. In yet another embodiment, the carrier is grapeseed oil.

In an embodiment, the flavoring agent is selected from the group consisting of catnip oil, chicken liver powder, poultry extract, maltodextrin, butter, and bacon. In another embodiment, the flavoring agent is chicken liver powder.

In an embodiment, the composition is formulated as a chew for oral administration. In another embodiment, the chew is produced using cold extrusion. In another embodiment, the weight of the chew is about 0.5-10 g. In yet another embodiment, the weight of the chew is about 4 g, about 6 g, about 9 g, or about 10 g. In still another embodiment, the weight of the chew is about 0.5 g. In an embodiment, the weight of the chew is about 1 g. In another embodiment, the weight of the chew is about 1.5 g. In yet another embodiment, the weight of the chew is about 2 g. In still another embodiment, the weight of the chew is about 3 g. In an embodiment, the weight of the chew is about 4 g. In another embodiment, the weight of the chew is about 5 g. In yet another embodiment, the weight of the chew is about 6 g. In still another embodiment, the weight of the chew is about 7 g. In an embodiment, the weight of the chew is about 8 g. In another embodiment, the weight of the chew is about 9 g. In yet another embodiment, the weight of the chew is about 10 g.

In an embodiment, the 4 g chew comprises:

about 7 mg of cannabidiol;

about 6 mg of cannabidiolic acid;

about 0.12 mg cannabigerolic acid;

about 0.32 mg Δ9-tetrahydrocannabinol; and

about 0.36 mg cannabichromene.

### Methods of Treatment

In an aspect, provided herein is a method for treating or reducing pain in a veterinary subject in need thereof, comprising administering to the subject a therapeutically effective amount of any of the compositions or dosage forms described above.

In an embodiment, the pain is associated with arthritis, post-operative pain, acute pain, dental pain, pain associated with gingivitis, joint pain, or multi-joint pain.

In an embodiment, the veterinary subject has cancer. In an embodiment, the cancer is a solid tumor, such as lung cancer, prostate cancer, colorectal cancer, thyroid cancer, renal cancer, adrenal cancer, liver cancer, pancreatic cancer, mammary cancer and central and peripheral nervous system cancer. In another embodiment, the cancer is a hematopoietic tumor, such as lymphomas and leukemias

In an embodiment, the veterinary subject is undergoing chemotherapy. In an embodiment, the chemotherapy is L-asparaginase, cyclophosphamide, doxorubicin, vincristine, and prednisolone (L-CHOP) or cyclophosphamide, doxorubicin, vincristine, and prednisolone (CHOP).

In an embodiment, the method results in a reduced tumor burden. In another embodiment, the method results in apoptosis of tumor cells. In another embodiment, the method results in a decrease in the proliferation of tumor cells.

In an embodiment, the pharmaceutical composition or dosage form is administered at a dosage of about 0.1-15.0 mg/kg. In another embodiment, the pharmaceutical composition or dosage form is administered at a dosage of about 0.1-10.0 mg/kg. In yet another embodiment, the pharmaceutical composition or dosage form is administered at a dosage of about 0.1 mg/kg. In still another embodiment, the pharmaceutical composition or dosage form is administered at a dosage of about 0.2 mg/kg. In yet another embodiment, the pharmaceutical composition or dosage form is administered at a dosage of about 0.3 mg/kg. In an embodiment, the pharmaceutical composition or dosage form is administered at a dosage of about 0.4 mg/kg. In another embodiment, the pharmaceutical composition or dosage form is administered at a dosage of about 0.5 mg/kg. In yet another embodiment, the pharmaceutical composition or dosage form is administered at a dosage of about 0.6 mg/kg. In still another embodiment, the pharmaceutical composition or dosage form is administered at a dosage of about 0.7 mg/kg. In yet another embodiment, the pharmaceutical composition or dosage form is administered at a dosage of about 0.8 mg/kg. In an embodiment, the pharmaceutical composition or dosage form is administered at a dosage of about 0.9 mg/kg. In another embodiment, the pharmaceutical composition or dosage form is administered at a dosage of about 1 mg/kg. In yet another embodiment, the pharmaceutical composition or dosage form is administered at a dosage of about 1.5 mg/kg. In still another embodiment, the pharmaceutical composition or dosage form is administered at a dosage of about 2 mg/kg. In an embodiment, the pharmaceutical composition or dosage form is administered at a dosage of about 3 mg/kg. In another embodiment, the pharmaceutical composition or dosage form is administered at a dosage of about 4 mg/kg. In yet another embodiment, the pharmaceutical composition or dosage form is administered at a dosage of about 5 mg/kg. In still another embodiment, the pharmaceutical composition or dosage form is administered at a dosage of about 6 mg/kg. In an embodiment, the pharmaceutical composition or dosage form is administered at a dosage of about 7 mg/kg. In another embodiment, the pharmaceutical composition or dosage form is administered at a dosage of about 8 mg/kg. In yet another embodiment, the pharmaceutical composition or dosage form is administered at a dosage of about 9 mg/kg. In still another embodiment, the pharmaceutical composition or dosage form is administered at a dosage of about 10 mg/kg. In an embodiment, the pharmaceutical composition or dosage form is administered at a dosage of about 11 mg/kg. In another embodiment, the pharmaceutical composition or dosage form is administered at a dosage of about 12 mg/kg. In yet another embodiment, the pharmaceutical composition or dosage form is administered at a dosage of about 13 mg/kg. In still another embodiment, the pharmaceutical composition or dosage form is administered at a dosage of about 14 mg/kg. In an embodiment, the pharmaceutical composition or dosage form is administered at a dosage of about 15 mg/kg.

In another embodiment, the pharmaceutical composition or dosage form is administered at twice the therapeutically effective dosage for one week, and then subsequently administered at a therapeutically effective dosage. In yet another embodiment, the therapeutically effective dosage is about 0.1-0.5 mg/kg. In still another embodiment, the therapeutically effective dosage is about 2 mg/kg. In an embodiment, the therapeutically effective dosage is about 8 mg/kg.

In an embodiment, the pharmaceutical composition or dosage form is administered at a dosage of about 1 mg/kg for one week, and then subsequently administered at a dosage of about 0.1-0.5 mg/kg. In another embodiment, the pharmaceutical composition or dosage form is administered at a dosage of about 4 mg/kg for one week, and then subsequently administered at a dosage of about 2 mg/kg.

In an embodiment, the pharmaceutical composition or dosage form is administered at a dosage of about 2 mg/kg every 12 hours for two weeks, then subsequently administered at a dosage of about 1 mg/kg every 12 hours for two weeks, and then subsequently administered at a dosage of about 2 mg/kg every 12 hours for four weeks.

In an embodiment, the pharmaceutical composition or dosage form is administered at a dosage of about 1.0 mg/kg once daily. In an embodiment, the pharmaceutical composition or dosage form is administered at a dosage of about 1.0 mg/kg twice daily. In an embodiment, the pharmaceutical composition or dosage form is administered at a dosage of about 1.0 mg/kg three times daily. In an embodiment, the pharmaceutical composition or dosage form is administered at a dosage of about 1.0 mg/kg four times daily.

In an embodiment, the pharmaceutical composition or dosage form is administered at a dosage of about 2.0 mg/kg once daily. In an embodiment, the pharmaceutical composition or dosage form is administered at a dosage of about 2.0 mg/kg twice daily. In an embodiment, the pharmaceutical composition or dosage form is administered at a dosage of about 2.0 mg/kg three times daily. In an embodiment, the pharmaceutical composition or dosage form is administered at a dosage of about 2.0 mg/kg four times daily.

In an embodiment, the pharmaceutical composition or dosage form is administered at a dosage of about 3.0 mg/kg once daily. In an embodiment, the pharmaceutical composition or dosage form is administered at a dosage of about 3.0 mg/kg twice daily. In an embodiment, the pharmaceutical composition or dosage form is administered at a dosage of about 3.0 mg/kg three times daily. In an embodiment, the pharmaceutical composition or dosage form is administered at a dosage of about 3.0 mg/kg four times daily.

In an embodiment, the pharmaceutical composition or dosage form is administered at a dosage of about 4.0 mg/kg once daily. In an embodiment, the pharmaceutical composition or dosage form is administered at a dosage of about 4.0 mg/kg twice daily. In an embodiment, the pharmaceutical composition or dosage form is administered at a dosage of about 4.0 mg/kg three times daily. In an embodiment, the pharmaceutical composition or dosage form is administered at a dosage of about 4.0 mg/kg four times daily. In an embodiment, the pharmaceutical composition or dosage form is administered at a dosage of about 5.0 mg/kg once daily. In an embodiment, the pharmaceutical composition or dosage form is administered at a dosage of about 5.0 mg/kg twice daily. In an embodiment, the pharmaceutical composition or dosage form is administered at a dosage of about 5.0 mg/kg three times daily. In an embodiment, the pharmaceutical composition or dosage form is administered at a dosage of about 5.0 mg/kg four times daily.

In an embodiment, the pharmaceutical composition or dosage form is administered at a dosage of about 6.0 mg/kg once daily. In an embodiment, the pharmaceutical composition or dosage form is administered at a dosage of about 6.0 mg/kg twice daily. In an embodiment, the pharmaceutical composition or dosage form is administered at a dosage of about 6.0 mg/kg three times daily. In an embodiment, the pharmaceutical composition or dosage form is administered at a dosage of about 6.0 mg/kg four times daily.

In an embodiment, the pharmaceutical composition or dosage form is administered at a dosage of about 7.0 mg/kg once daily. In an embodiment, the pharmaceutical composition or dosage form is administered at a dosage of about 7.0 mg/kg twice daily. In an embodiment, the pharmaceutical composition or dosage form is administered at a dosage of about 7.0 mg/kg three times daily. In an embodiment, the pharmaceutical composition or dosage form is administered at a dosage of about 7.0 mg/kg four times daily.

In an embodiment, the pharmaceutical composition or dosage form is administered at a dosage of about 8.0 mg/kg once daily. In an embodiment, the pharmaceutical composition or dosage form is administered at a dosage of about 8.0 mg/kg twice daily. In an embodiment, the pharmaceutical composition or dosage form is administered at a dosage of about 8.0 mg/kg three times daily. In an embodiment, the pharmaceutical composition or dosage form is administered at a dosage of about 8.0 mg/kg four times daily.

In an embodiment, the pharmaceutical composition or dosage form is administered at a dosage of about 9.0 mg/kg once daily. In an embodiment, the pharmaceutical composition or dosage form is administered at a dosage of about 9.0 mg/kg twice daily. In an embodiment, the pharmaceutical composition or dosage form is administered at a dosage of about 9.0 mg/kg three times daily. In an embodiment, the pharmaceutical composition or dosage form is administered at a dosage of about 9.0 mg/kg four times daily.

In an embodiment, the pharmaceutical composition or dosage form is administered at a dosage of about 10.0 mg/kg once daily. In an embodiment, the pharmaceutical composition or dosage form is administered at a dosage of about 10.0 mg/kg twice daily. In an embodiment, the pharmaceutical composition or dosage form is administered at a dosage of about 10.0 mg/kg three times daily. In an embodiment, the pharmaceutical composition or dosage form is administered at a dosage of about 10.0 mg/kg four times daily.

In an embodiment, the method results in a therapeutically effective median maximal serum concentration of cannabidiol. In another embodiment, the median maximal serum concentration of cannabidiol is about 90-310 ng/mL. In yet another embodiment, the median maximal serum concentration of cannabidiol is about 90 ng/mL. In still another embodiment, the median maximal serum concentration of cannabidiol is about 100 ng/mL. In still another embodiment, the median maximal serum concentration of cannabidiol is about 102 ng/mL. In an embodiment, the median maximal serum concentration of cannabidiol is about 200 ng/mL. In another embodiment, the median maximal serum concentration of cannabidiol is about 300 ng/mL. In yet another embodiment, the median maximal serum concentration of cannabidiol is about 400 ng/mL. In still another embodiment, the median maximal serum concentration of cannabidiol is about 500 ng/mL. In an embodiment, the median maximal serum concentration of cannabidiol is about 590 ng/mL. In another embodiment, the median maximal serum concentration of cannabidiol is about 600 ng/mL.

In an embodiment, the veterinary subject is canine, feline, bovine, porcine, or equine. In another embodiment, the veterinary subject is canine. In yet another embodiment, the veterinary subject is feline.

In an aspect, provided herein is a method for treating or reducing pain associated with arthritis, post-operative pain, acute pain, dental pain, pain associated with gingivitis, joint pain, or multi-joint pain in a veterinary subject in need thereof, comprising administering to the subject a therapeutically effective amount of hemp extract.

In an embodiment, the hemp extract is administered at a dosage of about 0.1-15.0 mg/kg. In another embodiment, the hemp extract is administered at a dosage of about 0.1-10.0 mg/kg. In yet another embodiment, the hemp extract is administered at a dosage of about 0.1 mg/kg. In still another embodiment, the hemp extract is administered at a dosage of about 0.2 mg/kg. In yet another embodiment, the hemp extract is administered at a dosage of about 0.3 mg/kg. In an embodiment, the hemp extract is administered at a dosage of about 0.4 mg/kg. In another embodiment, the hemp extract is administered at a dosage of about 0.5 mg/kg. In yet another embodiment, the hemp extract is administered at a dosage of about 0.6 mg/kg. In still another embodiment, the hemp extract is administered at a dosage of about 0.7 mg/kg. In yet another embodiment, the hemp extract is administered at a dosage of about 0.8 mg/kg. In an embodiment, the hemp extract is administered at a dosage of about 0.9 mg/kg. In another embodiment, the hemp extract is administered at a dosage of about 1 mg/kg. In yet another embodiment, the hemp extract is administered at a dosage of about 1.5 mg/kg. In still another embodiment, the hemp extract is administered at a dosage of about 2 mg/kg. In an embodiment, the hemp extract is administered at a dosage of about 3 mg/kg. In another embodiment, the hemp extract is administered at a dosage of about 4 mg/kg. In yet another embodiment, the hemp extract is administered at a dosage of about 5 mg/kg. In still another embodiment, the hemp extract is administered at a dosage of about 6 mg/kg. In an embodiment, the hemp extract is administered at a dosage of about 7 mg/kg. In another embodiment, the hemp extract is administered at a dosage of about 8 mg/kg. In yet another embodiment, the hemp extract is administered at a dosage of about 9 mg/kg. In still another embodiment, the hemp extract is administered at a dosage of about 10 mg/kg. In an embodiment, the hemp extract is administered at a dosage of about 11 mg/kg. In another embodiment, the hemp extract is administered at a dosage of about 12 mg/kg. In yet another embodiment, the hemp extract is administered at a dosage of about 13 mg/kg. In still another embodiment, the hemp extract is administered at a dosage of about 14 mg/kg. In an embodiment, the hemp extract is administered at a dosage of about 15 mg/kg.

In another embodiment, the hemp extract is administered at twice the therapeutically effective dosage for one week, and then subsequently administered at a therapeutically effective dosage. In yet another embodiment, the therapeutically effective dosage is about 0.1-0.5 mg/kg. In still another embodiment, the therapeutically effective dosage is about 2 mg/kg. In an embodiment, the therapeutically effective dosage is about 8 mg/kg.

In an embodiment, the hemp extract is administered at a dosage of about 1 mg/kg for one week, and then subsequently administered at a dosage of about 0.1-0.5 mg/kg. In another embodiment, the hemp extract is administered at a dosage of about 4 mg/kg for one week, and then subsequently administered at a dosage of about 2 mg/kg.

In an embodiment, the method results in a therapeutically effective median maximal serum concentration of cannabidiol. In another embodiment, the median maximal serum concentration of cannabidiol is about 90-310 ng/mL. In yet another embodiment, the median maximal serum concentration of cannabidiol is about 90 ng/mL. In still another embodiment, the median maximal serum concentration of cannabidiol is about 100 ng/mL. In still another embodiment, the median maximal serum concentration of cannabidiol is about 102 ng/mL. In an embodiment, the median maximal serum concentration of cannabidiol is about 200 ng/mL. In another embodiment, the median maximal serum concentration of cannabidiol is about 300 ng/mL. In yet another embodiment, the median maximal serum concentration of cannabidiol is about 400 ng/mL. In still another embodiment, the median maximal serum concentration of cannabidiol is about 500 ng/mL. In an embodiment, the median maximal serum concentration of cannabidiol is about 590 ng/mL. In another embodiment, the median maximal serum concentration of cannabidiol is about 600 ng/mL.

In an embodiment, the veterinary subject is administered gabapentin in combination with a dosage of hemp extract provided herein. In other embodiments, the veterinary subject is administered a dosage of about of about 1, 10, 15, 20, 25, 30, 35, 40, 45 or 50 mg/kg of gabapentin with a dosage of hemp extract provided herein. In another embodiment, the veterinary subject is administered a dosage of about 10 mg/kg of gabapentin with a dosage of hemp extract provided herein.

In another embodiment, the veterinary subject is administered a dosage of from 1 mg/kg to 50 mg/kg of gabapentin with a dosage of hemp extract provided herein. In another embodiment, the veterinary subject is administered a dosage from 10 mg/kg to 40 mg/kg of gabapentin with a dosage of hemp extract provided herein. In another embodiment, the veterinary subject is administered a dosage from 1 mg/kg to 20 mg/kg of gabapentin with a dosage of hemp extract provided herein. In another embodiment, the veterinary subject is administered a dosage from 5 mg/kg to 15 mg/kg of gabapentin with a dosage of hemp extract provided herein. In another embodiment, the veterinary subject is administered a dosage from 12 mg/kg to 14 mg/kg of gabapentin with a dosage of hemp extract provided herein.

In some embodiments, the veterinary subject is administered gabapentin about every 1, 2, 3, 4, 5, 6, 7, 8, 9, 10, 11 or 12 hours with a dosage of hemp extract provided herein. In one embodiment, the veterinary subject is administered gabapentin about every 8 hours with a dosage of hemp extract provided herein. The hemp extract can be administered with the gabapentin or at a different time and/or on a different schedule.

In another embodiment, the veterinary subject is administered gabapentin 1, 2, 3, 4, 5 or 6 times daily with a dosage of hemp extract provided herein. The hemp extract can be administered with the gabapentin or at a different time and/or on a different schedule.

In an embodiment, the veterinary subject is administered about 10 mg/kg gabapentin about every 8 hours with a dosage of hemp extract provided herein. The hemp extract can be administered with the gabapentin or at a different time and/or on a different schedule. In another embodiment, the veterinary subject is administered 10 mg/kg gabapentin and 8 mg/kg hemp extract every 8 hours. The hemp extract can be administered with the gabapentin or at a different time.

In an embodiment, the veterinary subject is canine, feline, bovine, porcine, or equine. In another embodiment, the veterinary subject is canine. In yet another embodiment, the veterinary subject is feline.

The pharmaceutical compositions and dosage forms of the present disclosure may be administered by any convenient route, for example, by infusion or bolus injection, by absorption through epithelial or mucocutaneous linings (e.g., oral mucosa, rectal and intestinal mucosa, etc.) and may be administered together with any other therapeutic agent. Administration can be systemic or local. In an embodiment, administration is topical. In another embodiment, topical administration is used to treat local pain. In another embodiment, the local pain is joint pain. In an embodiment, the veterinary subject is an animal >100 kg (e.g., a horse, cow, or pig).

The therapeutic compositions of the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose may vary depending upon the age and the weight of a subject to be administered, target disease, conditions, route of administration, and the like. Various delivery systems are known and can be used to administer the pharmaceutical composition of the invention, e.g., encapsulation in liposomes, microparticles, microcapsules, receptor mediated endocytosis (see, e.g., Wu et al. (1987) J. Biol. Chem. 262:4429-4432). Methods of introduction include, but are not limited to, intradermal, intramuscular, intraperitoneal, intravenous, transdermal, buccal, sublingual, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum, methyl cellulose, hydroxypropylmethyl-cellulose, sodium carbomethylcellulose, and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures.

The injectable preparations may include dosage forms for intravenous, subcutaneous, intracutaneous and intramuscular injections, local injection, drip infusions, etc. These injectable preparations may be prepared by methods publicly known. For example, the injectable preparations may be prepared, e.g., by dissolving, suspending or emulsifying the pharmaceutical composition or dosage form in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active components may be dissolved or suspended in suitable liquids, such as fatty oils, liquid paraffin, or liquid polyethylene glycols.

Alternatively, the composition may be in a powder form for constitution before use with a suitable vehicle, e.g., sterile, pyrogen-free water. The exact formulation, route of administration and dosage may be chosen by the physician familiar with the patient's condition. (See for example Fingl, et al., 1975, in “The Pharmacological Basis of Therapeutics”, Chapter I, p. 1). Depending on the severity and responsiveness of the condition treated, dosing can also be a single administration of a slow release composition, with course of treatment lasting from several days to several weeks or until cure is effected or diminution of the disease state is achieved.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, chews, pet food, etc. In certain embodiments, for the dosages provided above, they are administered in one serving of pet food, e.g. 1 mg/kg of hemp extract provided in one serving of pet food.

In accordance with the methods disclosed herein, pharmaceutical formulations can be administered to the patient using any acceptable device or mechanism. For example, the administration can be accomplished using a syringe and needle or with a reusable pen and/or autoinjector delivery device. The methods of the present invention include the use of numerous reusable pen and/or autoinjector delivery devices to administer a pharmaceutical formulation.

In an embodiment for non-human animal administration, the term “pharmaceutical” as used herein may be replaced by “veterinary.”

### EXAMPLES

### Example 1: CBD Oil and Protocols Approval

The industrial hemp strain used in this study was a proprietary hemp strain utilizing ethanol and heat extraction with the final desiccated product reconstituted into an olive oil base containing approximately 10 mg/ml of CBD as an equal mix of CBD and carboxylic acid of CBD (CBDa), 0.24 mg/ml tetrahydrocannabinol (THC), 0.27 mg/ml cannabichromene (CBC), and 0.11 mg/ml cannabigerol (CBG) which is dehydrated; all other cannabinoids were less than 0.01 mg/ml. Analysis of 5 different production runs using a commercial analytical laboratory (MCR Laboratories, Framingham, MA) show less than a 9% difference across batches for each of the detected cannabinoids listed above. The study was performed after the Cornell University institutional animal care and use committee (IACUC) approved the study which follows the guidelines for animal use according to the IACUC. Client owned dogs were enrolled after informed consent in accordance with the Declaration of Helsinki.

### Example 2: Terpene Profiles

Terpene profiles were determined using gas chromatography with flame ionization detection (GC-FID) analysis of headspace for four separate oil extractions. All oils contained 0.09-0.13% α-pinene, 0.23-0.44% β-myrcene, 0.04-0.09% β-pinene, 0.05-0.09% δ-limonene, 0.03-0.06% linalool, 0.04-0.07% β-caryophyllene, 0.02-0.04% α-humulene, 0.04-0.07% nerolidol 2, 0.02-0.04% guaiol, 0.04-0.08% caryophyllene oxide, and 0.01-0.04% α-bisabolol. In addition, some of the oils tested contained 0.02% camphene, 0.02-0.03% β-ocimene, 0.02-0.05% eucalyptol, 0.02% isopulegol, and/or 0.02-0.04% nerolidol 1. Total terpenes ranged from 0.73-1.10%,

### Example 3: Pharmacokinetics

An initial investigation into single-dose oral pharmacokinetics was performed with 4 beagles (3.5-7 years, male castrated, 10.7-11.9 kg). Each dog received a 2 mg/kg and an 8 mg/kg oral dosage of CBD oil, with a 2-week washout period between each experiment. The dogs were fed two hours after dosing. Physical examination was performed at 0, 4, 8 and 24 hours after dosing. Attitude, behavior, proprioception, and gait were subjectively evaluated at each time point during free running/walking and navigation around standard traffic cones (weaving). Five ml of blood was collected at time 0, 0.5, 1, 2, 4, 8, 12 and 24 hours after oil administration. Blood samples were obtained via jugular venipuncture and transferred to a coagulation tube for 20 minutes. Samples were centrifuged (VWR, Clinical Centrifuge) at 3,600×g for 10 minutes; serum was removed and stored at −80° C. until analysis using liquid chromatography-mass spectrometry (LC-MS) at Colorado State University Core Mass Spectrometry facility.

### Example 4: Extraction of CBD from Canine Serum and Mass Spectrometry Analysis

CBD was extracted from canine serum using a combination of protein precipitation and liquid-liquid extraction using n-hexane, with minor modifications for microflow ultra-high pressure liquid chromatography (UHPLC). Briefly, 0.05 ml of canine serum was subjected to protein precipitation in the presence of ice-cold acetonitrile (80% final concentration), spiked with deuterated CBD as the internal standard (0.06 mg/ml, CDB-d₃ Cerilliant, Round Rock, Tex., USA). 0.2 ml of water was added to each sample prior to the addition of 1.0 ml of hexane to enhance liquid-liquid phase separation. Hexane extract was removed and concentrated to dryness under laboratory nitrogen. Prior to LC-MS analysis, samples were resuspended in 0.06 mL of 100% acetonitrile. A standard curve using the CBD analytical standard was prepared in canine serum non-exposed to CBD and extracted as above. Cannabidiol concentration in serum was quantified using a chromatographically coupled triple-quadropole mass spectrometer (UHPLC-QQQ-MS).

### Example 5: CBD Serum Concentration Data Analysis

From the UHPLC-QQQ-MS data, peak areas were extracted for CBD detected in biological samples and normalized to the peak area of the internal standard CBD-d₃, in each sample using Skyline as well as an in-house R Script (www.r-project.org). CBD concentrations were calculated to nanograms per mL of serum as determined by the line of regression of the standard curve (r2=0.9994, 0-1000 ng/mL). For this assay, the limits of detection (LOD) and limits of quantification (LOQ) represent the lower limits of detection and quantification for each compound in the matrix of this study. Pharmacokinetic variables were estimated by means of non-compartmental analysis, utilizing a pharmacokinetic software package (PK Solution, version 2.0, Montrose, Colo., USA).

### Example 6: Inclusion and Exclusion Criteria for Clinical Trial

The study population consisted of client-owned dogs presenting to Cornell University Hospital for Animals for evaluation and treatment of a lameness due to OA. Dogs were considered for inclusion in the study if they had radiographic evidence of OA, signs of pain according to assessment by their owners, detectable lameness on visual gait assessment and painful joint(s) on palpation. Each dog had an initial complete blood count ([CBC] Bayer Advia 120, Siemens Corp., New York, N.Y., USA) and serum chemistry analysis (Hitachi 911, Roche Diagnostics, Indianapolis, Ind., USA) performed to rule out any underlying disease that might preclude enrolment. Elevations in alkaline phosphatase (ALP), alanine aminotransferase (ALT) and aspartate aminotransferase (AST) were allowed if prior hepatic ultrasound was deemed within normal limits except for potential non-progressive nodules (possible hepatic nodular hyperplasia).

All owners completed a brief questionnaire to define the affected limb(s), duration of lameness, and duration of analgesic or other medications taken.

All dogs underwent radiographic examination of affected joints and a radiologist confirmed the presence or absence of OA, and excluded the presence of concomitant disease that might preclude them from enrolment (i.e. lytic lesions).

During the trial, dogs were only allowed to receive NSAIDs, fish oil, and/or glucosamine/chondroitin sulphate without any change in these medications for 4 weeks prior to or during the 10-week study period as standard of care for the disease process. Other analgesic medications used, such as gabapentin and tramadol, were discontinued at least 2 weeks prior to enrollment. Dogs were excluded if they had evidence of renal, uncontrolled endocrine, neurologic, or neoplastic disease, or if they had a temperament not suited for gaiting on a lead or were undergoing physical therapy. Every dog was fed its regular diet with no change allowed during the trial.

### Example 7: Clinical Trial

The study was a placebo-controlled, double-blind, cross-over clinical trial. Dogs received each of two treatments in random order (Randomizer iPhone Application): CBD, 2 mg/kg every 12 hours, or placebo (an equivalent volume of olive oil with 10 parts per thousands of anise oil and 5 parts per thousands of peppermint oil to provide a similar herbal smell) every 12 hours. Each treatment was administered for 4 weeks with a 2-week washout period in between treatments. Blood was collected to repeat complete blood counts and chemistry analysis at weeks 2 and 4 for each treatment.

At each visit, each dog was evaluated by a veterinarian based on a scoring system, as well as by its owner (canine brief pain inventory [CBPI], Hudson activity scale) before treatment initiation and at weeks 2 and 4 thereafter.

### Example 8: Statistical Analysis

Initial power analysis was performed to assess number of dogs needed for this study as a cross over design with a power set 0.80 and alpha of 0.05 using prior data suggesting a baseline CBPI or Hudson score change of approximately 15 points (two tailed) with a standard deviation of 20. When calculated it was assumed that 14 dogs would be necessary to find significance.

Statistical analysis was performed with a commercially available software package (JMP 12.0, Cary, N.C., USA). All data was assessed utilizing a Shapiro-Wilks test for normality. Considering a majority of our blood, serum and scoring data was normally distributed a mixed model analysis of variance was used. Cross-over study variables included in the model were: fixed effects of treatment, time, sequence of oil, gender, age, NSAID usage, treatment×time; as well as random effects of observation period, period nested within dog, time point nested within period nested within dog. To control for difference and relative change in CBPI pain and activity interference assessments and Hudson scoring across dogs, the fixed effect of initial CPBI or Hudson Score was also included for these analyses. Dunnett's tests were performed post hoc on any significant effects of time x treatment to assess differences with week 0 of CBD oil or placebo oil as the baseline time point for comparison. A p value of less than 0.05 was determined to be significant for all analyses.

### Example 9: Pharmacokinetic Results

Pharmacokinetics demonstrated that CBD half-life of elimination median was 4.2 hours (3.8-6.8 hours) with the 2 mg/kg dose, and 4.2 hours (3.8-4.8 hours) with the 8 mg/kg dose (Table 1). Median maximal concentration of CBD oil (FIG. 2) was 102.3 mg/ml (60.7-132.0 ng/mL; 180 nM) and 590.8 ng/mL (389.5-904.5 ng/mL; 1.2 uM) and was reached after 1.5 hours and 2 hours, respectively, for 2 and 8 mg/kg doses. No obvious psychoactive properties were observed on evaluation at any time point during the 2 and 8 mg/kg doses over 24 hours. These results led to a practical dosing during the clinical trial of 2 mg/kg body weight every 12 hours.

TABLE 1
Serum pharmacokinetic of 2 mg/kg and 8 mg/kg oral dosage of CBD oil
medians and ranges after 2 mg/kg and 8 mg/kg single oral dosing

| Dose (2 mg/kg) | Cmax (ng/ml) | Tmax (h) | T1/2 elim (h) | AUC 0-t (ng-hr/ml) | MRT(h) |
|---|---|---|---|---|---|
| Dog 1 | 60.7 | 1 | 4.4 | 183 | 6 |
| Dog 2 | 132 | 1 | 3.9 | 351 | 4.2 |
| Dog 3 | 102.7 | 2 | 3.8 | 382 | 5.1 |
| Dog 4 | 101.9 | 2 | 6.8 | 437 | 9.1 |
| Median (Range) | 102.3 (60.7, 132.0) | 1.5 (1.0-2.0) | 4.2 (3.8-6.8) | 367.2 (1833437.4) | 5.6 (4.2-9.1) |
| Dose (8 mg/kg) | Cmax (ng/ml) | Tmax (h) | T1/2 elim (h) | AUC 0-t (ng-hr/ml) | MRT(h) |
| Dog 1 | 499 | 2 | 3.8 | 2928 | 5.7 |
| Dog 2 | 389 | 1 | 4.8 | 1753 | 7 |
| Dog 3 | 904 | 2 | 4.2 | 3048 | 5.1 |
| Dog 4 | 682 | 2 | 4.1 | 2389 | 5.2 |
| Median (Range) | 590.8 (389-5, 604.5) | 2.0 (1.0-2.0) | 4.2 (3.84.8) | 2658.6 (1753.6-3048.6) | 5.6 (5.1-7.0) |

Legend:
Cmax = maximum concentration;
Tmax = time of maximum concentration;
T1/2 el = half-life of elimination;
AUC 0-t = area under the curve (time 0 to 24 h);
MRT = median residence time.

### Example 10: Dogs Included in Clinical Trial

Twenty-two client-owned dogs with clinically and radiographically confirmed evidence of osteoarthritis were recruited. Sixteen of these dogs completed the trial and were included in the analyses; their breed, weight, age, sex, worse affected limb, radiographic findings, use of NSAIDs and sequence of treatments are summarized in Table 2. Dogs were removed due to osteosarcoma at the time of enrolment, gastric torsion (placebo), prior aggression issues (CBD oil), pyelonephritis/kidney insufficiency (CBD oil), recurrent pododermatitis (placebo oil), and diarrhea (placebo oil).

TABLE 2
Characteristics (breed, weight, age, sex, affected limbs, radiographic
findings, concomitant utilization of NSAID and sequence of treatment) of the dogs
included in this study.

| | Weight | Age | | Worse | | |
|---|---|---|---|---|---|---|
| Breed | (kg) | (years) | Sex | limb | Radiographic findings and OA localization | NSAID |
| Rottweiler | 35.3 | 10 | FS | | Moderate, intracapsular swelling with moderate | Carprofen |
| | | | | | osteophytosis, left stifle | (2.1 mg/kg BID) |
| Mix | 30.6 | 13 | MC | RF | Moderste-to-severe, right-shoulder osteoarthrosis; | No |
| | | | | | mild, left shoulder osteoarthrosis | |
| | | | | | Moderate-to-sever, bilateral hip osteoarthrosis | |
| Mix | 27.2 | 9 | FS | LF | Moderate medial coronoid remodeling (with | No |
| | | | | | fragmentation on the right) and bilateral elbow | |
| | | | | | osteoarthrosis | |
| Mix | 30.5 | 14 | MC | | Moderate enthesopathies on right carpus; mild, left- | No |
| | | | | | antebrachiocarpal osteoarthrosis | |
| | | | | | Bilateral moderate coxofemoral osteoarthrosis | |
| Mix | 23.5 | 10 | FS | | Moderate bilateral stifle osteoarthrosis and moderate | Carprofen |
| | | | | | intracapsular swelling | (2.2 mg/kg) |
| Mix | 28.1 | 10 | FS | LF | Moderate bilateral elbow osteoarthrosis | Metacam |
| | | | | | Moderate left-stifle osteoarthrosis with intracapsular | (0.1 mg/kg) |
| | | | | | swelling | |
| English | 25.2 | 8 | MC | LF | Severe osteoarthrosis, left elbow | Vetprofen |
| Bulldog | | | | | Moderate intracapsular swelling and mild | (2.0 mg/kg BSD) |
| | | | | | osteoarthrosis, right stille | |
| German | 21.5 | 14 | FS | RH | Moderate bilateral elbow osteoarthrosis | Carprofen |
| Shorthaired | | | | | | (2.4 Mg/kg BID) |
| Pointer | | | | | | |
| Labrador | 26.1 | 13 | FS | | Bilateral sever stifle osteoarthrosis due to cranial | Metacam |
| Retriever | | | | | cruciate ligament disease | (0.1 mg/kg SID) |
| Mix | 18.2 | 13 | FS | RF | Bilateral moderate elbow osteoarthrosis and medial | Metacam |
| | | | | | epicondylitis | (0.1 mg/kg SID) |
| Mix | 22 | 9 | FS | RH | Moderate, stifle osteoarthrosis, medial coronoid | No |
| | | | | | disease, and medial epicondylitis | |
| Bernese | 50 | 3 | M | FS | Bilateral severe elbow osteoarthrosis, medial coronoid | Carprofen |
| Mountain | | | | | disease, and medial epicondylitis | (2.0 mg/kg SID) |
| Dog | | | | | | |
| Belgian | 25.1 | 9 | FS | RF | Severe bilateral elbow osteoarthrosis | Carprofen |
| Malinois | | | | | Bilateral moderate hip osteoarthrosis | (2 mg/kg BID) |
| Mix | 28.5 | 13 | FS | | Severe bilateral elbow osteoarthrosis | No |
| | | | | | Severe bilateral hip osteoarthrosis | |
| Border Collie | 22 | 14 | MC | | Severe thoracolumnbosacral osteoarthrosis | No |
| | | | | | Multifocal carpal enthesiophytes | |
| Beagle | 17.6 | 5 | MC | | Mild left elbow osteoarthrosis, with possible medial | No |
| | | | | | coronoid disease | |
| | | | | | Moderate-to-severe bilateral stifle osteoarthrosis | |

### Example 11: Clinical Trial Results

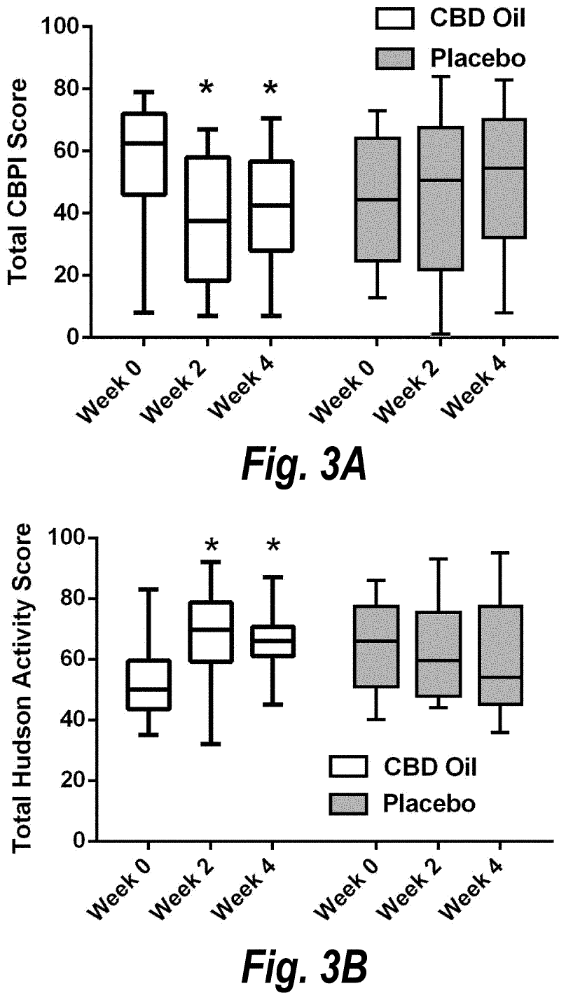

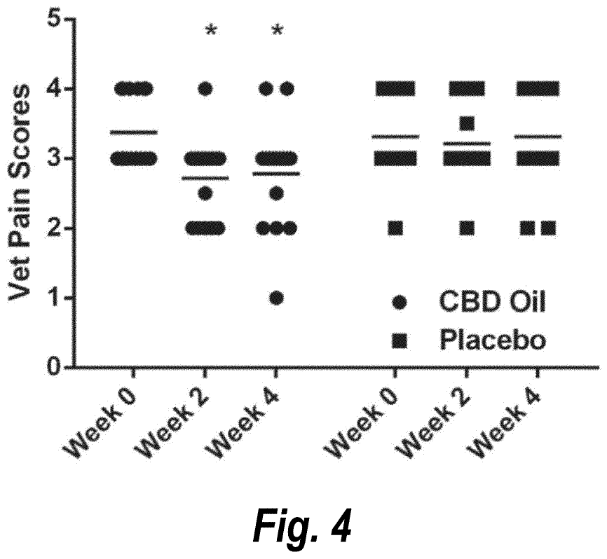

CBPI and Hudson scores (FIG. 3A and FIG. 3B) showed a significant decrease in pain and increase in activity (p<0.01) at week 2 and 4 during CBD treatment when compared to baseline week 0, while placebo treatment showed no difference in CBPI and Hudson scoring from scores prior to initiation of treatments (Table 3). Lameness as assessed by veterinarians (FIG. 4) showed an increase in lameness with age (p<0.01), whereas NSAID use (p=0.03) results in significantly less lameness. Veterinary pain scores showed significantly less pain in dogs on NSAIDs (p<0.01). CBD oil resulted in significantly less pain when compared to baseline on evaluation at both week 2 and week 4 (p<0.03), while 24 placebo treatment showed no significant differences. No changes were observed in weight-bearing capacity when evaluated utilizing the veterinary lameness and pain scoring system (Table 3).

TABLE 3
Average and standard deviation for CBPI and Hudson; median and range for
lameness, weight-bearing and pain scores at each time for treatment and placebo oils

| | Treatment A/CBD oil | | | Treatment B/placebo oil | | |
|---|---|---|---|---|---|---|
| | Week 0 | Week 2 | Week 4 | Week 0 | Week 2 | Week 4 |
| CBPI Pain (0-40) | 21 + 8 | 14 ± 6* | 14 ± 8* | 17 ± 7 | 19 ± 9 | 19 ± 9 |
| CBPI Interference | 35 + 15 | 25 + 15* | 26 + 14* | 27 + 15 | 29 + 15 | 31 ± 16 |
| (0-60) | | | | | | |
| Hudson (0-110) | 54 ± 13 | 67 ± 15* | 67 ± 10* | 65 ± 14 | 64 ± 16 | 60 ± 19 |
| Veterinary | 3 (1-4) | 3 (1-2) | 3 (1-4) | 3 (2-4) | 3 (2-4) | 3 (1-4) |
| lameness § | | | | | | |
| Veterinary pain ∫ | 3 (3-4) | 3 (2-4)* | 3 (1-0* | 3 (2-4) | 3 (2-4) | 3 (2-4) |
| Veterinary | 2 (1-3) | 2 (1-3) | 2 (1-3) | 2 (1-3) | 2 (1-3) | 2 (1-3) |
| weight-bearing [symbol] | | | | | | |

Legend:
Asterisk (*) represents significant difference (p < 0.05) from baseline week 0 of CBD treatment.
§ Lameness was scored as follows: 1 = no lameness observed/walks normally, 2 = slightly lame when walking, 3 = moderately lame when walking, 4 = severely lame when walking, 5 = reluctant to rise and will not walk more than 5 paces.
∫ Pain on palpation was scored as follows: 1= none, 2 = mild signs, dog turns head in recognition, 3 = moderate signs, dog pulls limb away, 4 = severe signs, dog vocalizes or becomes aggressive, 5 = dog will not allow alpation. [symbol]
Weight-bearing was scored as follows: 1 = equal on all limbs standing and walking, 2 = normal standing, favors affected limb when walking, 3 = partial weight-bearing standing and walking, 4 = partial weight-bearing standing, non-weight-bearing walking, 5 = non-weight-bearing standing and walking.

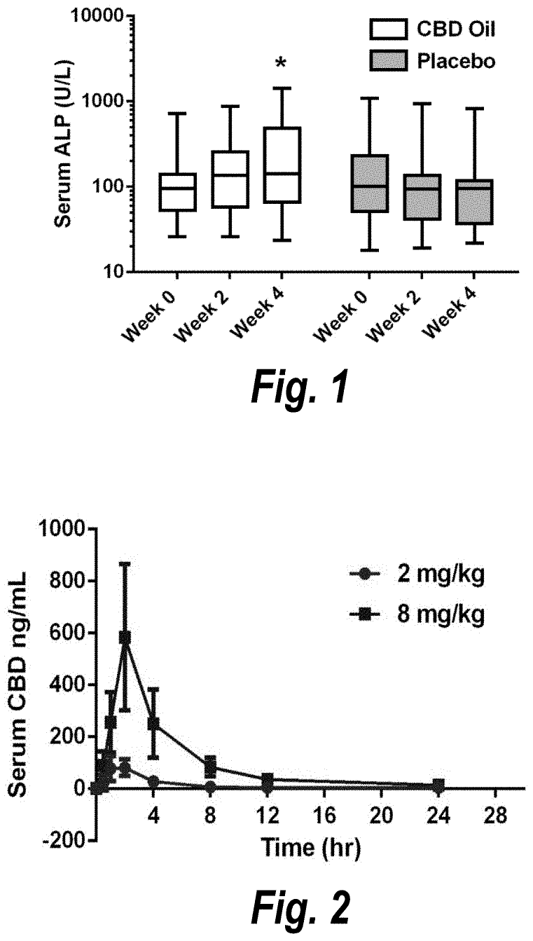

Chemistry analysis and CBC were performed at each visit. No significant change in the measured CBC values was noted in either the CBD oil or placebo treated dogs (data not shown). Serum chemistry values were not different between placebo compared to CBD oil (Table 4), except for alkaline phosphatase (ALP) which significantly increased over time from baseline by week 4 of CBD oil treatment (p=0.005); with nine of the sixteen dogs showing increases over time (FIG. 1). Glucose was increased in dogs receiving the placebo oil at each time point (p=0.04) and creatinine levels increased over time in both dogs receiving CBD oil and those receiving placebo oil (p<0.01); though all values remained within reference ranges. Other notable significances in serum chemistry values were associated with primarily age or NSAID use. An increase in age was associated with significantly higher blood urea nitrogen (BUN; p<0.001), calcium (p=0.014), phosphorus (p=0.001), alanine aminotransferase (ALT; p=0.028), ALP (p=0.012), gamma glutamyltransferase (GGT; p=0.018), globulin (p=0.021) and cholesterol (p=0.002) values. NSAID use was associated with significantly higher BUN (p=0.003), and creatinine (p=0.017), and significant decreases in total protein (p<0.001) and serum globulin (p<0.001).

TABLE 4
Mean ± SD values for serum chemistry data obtained at each time point for
dogs receiving CBD and placebo oils

| | | | Treatment A/CBD oil | | | Treatment B/placebo oil | | |
|---|---|---|---|---|---|---|---|---|
| | Reference | | Week 0 | Week 2 | Week 4 | Week 0 | Week 2 | Week 4 |
| Na | 145-153 | mEq/L | 149 ± 3 | 149 ± 2 | 149 ± 1 | 149 ± 1 | 149 ± 2 | 149 ± 2 |
| K | 4.1-5.6 | mEq/L | 4.9 ± 0.3 | 4.9 ± 0.5 | 4.9 ± 0.3 | 4.8 ± 0.4 | 4.9 ± 0.4 | 4.9 ± 0.3 |
| Cl | 105-116 | mEq/L | 110 ± 3 | 109 ± 3 | 109 ± 2 | 110 ± 2 | 110 ± 2 | 110 ± 2 |
| BUN | 10-32 | mg/dL | 20 ± 9 | 20 ± 7 | 20 ± 6 | 19 ± 6 | 21 ± 7 | 19 ± 6 |
| Creat | 0.6-1.4 | mg/dL | 1.0 ± 0.3 | 1.1 ± 0.3* | 1.0 ± 0.3′ | 0.9 ± 0.3 | 1.0 ± 0.3* | 1.0 ± 0.3* |
| Ca | 9.3-11.4 | mg/dL | 10.4 ± 0.5 | 10.4 ± 0.4 | 10.3 ± 0.4 | 10.4 ± 0.6 | 10.4 ± 0.4 | 10.4 ± 0.4 |
| P | 2.9-5.2 | mg/dL. | 3.8 ± 0.8 | 3.9 ± 0.8 | 3.9 ± 0.6 | 4.0 ± 0.7 | 3.9 ± 0.6 | 4.0 ± 0.5 |
| Mg | 1.4-2.2 | mg/dL | 1.8 ± 0.2 | 1.8 ± 0.2 | 1.8 ± 0.2 | 1.8 ± 0.1 | 1.8 ± 0.1 | 1.8 ± 0.1 |
| GLU | 63-118 | mg/dL | 92 ± 9 | 89 ± 9 | 92 ± 9 | 97 ± 10* | 93 ± 8 | 97 ± 10* |
| ALT | 20-98 | U/L | 93 ± 86 | 93 ± 88 | 114 ± 119 | 90 ± 89 | 222 ± 606 | 166 ± 284 |
| AST | 14-51 | U/L | 31 ± 8 | 33 ± 13 | 34 ± 16 | 30 ± 8 | 56 ± 99 | 45 ± 34 |
| ALP | 17-111 | U/L | 160 ± 212 | 238 ± 268 | 323 ± 407* | 204 ± 287 | 186 ± 287 | 175 ± 248 |
| GGT | 0-6 | U/L | 4 ± 3 | 3 ± 2 | 3 ± 2 | 3 ± 2 | 4 ± 6 | 5 ± 4 |
| TB | 0.0-0.2 | mg/dL | 0.1 ± 0.1 | 0.0 ± 0.1 | 0.1 ± 0.1 | 0.0 ± 0.1 | 0.0 ± 0.1 | 0.0 ± 0.1 |
| TP | 5.3-7.0 | g/dL | 6.3 ± 0.4 | 6.4 ± 0.5 | 6.3 ± 0.4 | 6.3 ± 0.4 | 6.3 ± 0.4 | 6.3 ± 0.4 |
| ALB | 3.1-4.2 | g/dL | 3.7 ± 0.2 | 3.7 + 0.2 | 3.7 ± 0.2 | 3.7 ± 0.2 | 3.7 ± 0.2 | 3.7 ± 0.2 |
| GLOB | 1.9-3.6 | g/dL | 2.6 ± 0.3 | 2.6 = 0.4 | 2.6 ± 0.4 | 2.6 = 0.4 | 2.6 ± O.4 | 2.6 ± 0.4 |
| CHOL | 138-332 | mg/dL | 291 ± 64 | 301 ± 62 | 302 ± 62 | 295 ± 71 | 300 ± 71 | 308 ± 83 |
| CK | 48-260 | U/L | 148 ± 81 | 147 ± 59 | 134 ± 61 | 139 ± 57 | 158 ± 80 | 168 ± 105 |

Legend:
Asterisk (*) indicates significantly different (p < 0.05) serum concentration from baseline week 0 CBD treatment.

### Example 12: Canine Safety Study

A 12-week safety study was performed in canines to evaluate the safety of a soft chew containing CBD.

### Animals and Study Design

Eight purebred beagle dogs, 11 months-5 years old, weighing 7.39-11.95 kg at study start were selected for the study, as shown in Table 5.

TABLE 5
Animal information

| Dog ID | Sex | Date of Birth |
|---|---|---|
| 13536 | F | Dec, 24, 2013 |
| 2753822 | F | Jan. 4, 2015 |
| 2908987 | F | Mar. 8, 2015 |
| 13644 | M | Feb. 7, 2017 |
| 2784123 | M | Feb. 8, 2015 |
| 2963028 | M | Sep. 12, 2015 |
| 13513 | F | Jul. 31, 2013 |
| 13490 | M | Nov. 1, 2012 |

Dogs were single housed in cages of a size in accordance with the Animal Welfare Act, with a 12-hour-light/12-hour-dark cycle and targeted conditions of 50° to 85° F. Cages and food bowls were cleaned daily and sanitized in accordance with the Animal Welfare Act. Fresh tap water, fit for human consumption, was available ad libitum by means of an automatic watering system. There were no known contaminants that were reasonably expected to be present in the dietary material that were known to be capable of interfering with the purpose or conduct of the study.

During the study, the control diet, Purina Dog Chow, was the sole source of food supplied to each animal once daily for approximately 1 hour. Dogs were fed according to ideal body condition and fasted for a minimum of 12 hours prior to blood collections. CBD was administered by a soft chew offered twice daily at the approximate dosage of 2 mg/kg. Dosing is shown in Table 6.

TABLE 6
Quantity of chews offered per week

| | | Week | | | | | |
|---|---|---|---|---|---|---|---|
| Dog ID | Sex | 1 | 2 | 3 | 4 | 5 | 6 |
| 13536 | F | 2 small | 1 large, 1 small | 1 large, 1 small | 1 large, 1 small | 1 large, 1 small | 1 large, 1 small |
| 2753822 | F | 1 large | 1 large | 1 Large | 1 large | 1 large | 1 large |
| 3808987 | F | 2 small | 2 small | 2 small | 2 small | 1 large, 1/2 small | 1 large, 1/2 small |
| 13644 | M | 1 large, 1/2 small | 1 large, 1/2 small | 1 large, 1/2 small | 1 large, 1/2 small | 1 large, 1/2 small | 1 large, 1/2 small |
| 2784123 | M | 1 large, 1/2 small | 1 large, 1/2 small | 1 large, 1/2 small | 1 large, 1/2 small | 1 large, 1/2 small | 1 large, 1/2 small |
| 2963028 | M | 1 large, 1/2 small | 1 large, 1/2 small | 1 large, 1/2 small | 1 large, 1/2 small | 1 large, 1/2 small | 1 large, 1/2 small |
| 13513 | F | 1 large, 1/2 small | 1 large, 1/2 small | 1 large, 1/2 small | 1 large, 1/2 small | 1 large, 1/2 small | 1 large, 1/2 small |
| 13490 | M | 2 small | 2 small | 2 small | 1 large, 1/2 small | 1 large, 1/2 small | 1 large, 1/2 small |
| | | Week | | | | | |
| Dog ID | Sex | 7 | 8 | 9 | 10 | 11 | 12 |
| 13536 | F | 1 large, 1 small | 1 large, 1 small | 1 large, 1 small | 1 large, 1 small | 1 large, 1 small | 1 large, 1 small |
| 2753822 | F | 1 large | 1 large | 1 Large | 1 large | 1 large | 1 large |
| 3808987 | F | 1 large, 1/2 small | 1 large, 1/2 small | 1 large, 1/2 small | 1 large, 1/2 small | 1 large, 1/2 small | 1 large, 1/2 small |
| 13644 | M | 1 large, 1/2 small | 1 large, 1/2 small | 1 large, 1/2 small | 1 large, 1/2 small | 1 large, 1/2 small | 1 large, 1/2 small |
| 2784123 | M | 1 large, 1/2 small | 1 large, 1/2 small | 1 large, 1/2 small | 1 large, 1/2 small | 1 large, 1/2 small | 1 large, 1/2 small |
| 2963028 | M | 1 large, 1/2 small | 1 large, 1/2 small | 1 large, 1/2 small | 1 large, 1/2 small | 1 large, 1/2 small | 1 large, 1/2 small |
| 13513 | F | 1 large, 1/2 small | 1 large, 1/2 small | 1 large, 1/2 small | 1 large, 1/2 small | 1 large, 1/2 small | 1 large, 1/2 small |
| 13490 | M | 1 large, 1/2 small | 1 large, 1/2 small | 1 large, 1/2 small | 1 large, 1/2 small | 1 large, 1/2 small | 2 small |

### CBC and Serum Chemistry

Prior to study initiation, 5 milliliters of blood was collected for each dog and was used to determine eligibility for the study. During the study, 5 milliliters of blood was collected weekly (±2 days). Blood was collected via jugular venipuncture in sterile syringes. Samples were split into two tubes: a red-top serum separator tube and a lavender-top EDTA tube. Red-top tubes were spun in a refrigerated centrifuge for 15 minutes at 3000 RPM after being allowed to clot. Lavender-top tubes were placed on a rocker to allow the blood to adequately mix with the anticoagulant. Blood samples were packaged and sent by priority-overnight to Antech Diagnostics for analysis.

### Pharmacokinetic (PK) Blood Collection

On the first day of dosing, blood was collected for a PK analysis from 6 of the 8 dogs. The most cooperative dogs were selected for the PK analysis. Approximately 6 milliliters of blood was collected via jugular venipuncture in sterile syringes at 0 min, 30 min, 60 min, 2 hrs, 4 hrs, 8 hrs, 12 hrs, and 24 hrs after treatment. Samples were placed into red top clotting tubes with no serum separator. Serum was harvested by centrifuging the tubes at 3000 RPM for 15 minutes. The harvested serum was placed in cyrovials and stored at −70° C. Each tube was labeled with the dog id, date of collection, and collection time point. Samples were shipped overnight on dry ice to the Proteomics & Metabolomics Facility at the Colorado State University.

### Clinical Observations

A veterinarian performed a complete physical examination of all dogs prior to the initiation of the study and at study completion. Each dog was evaluated as to general health, body and hair coat condition. Qualified personnel performed clinical observations twice daily in accordance with Summit Ridge Farms' Program of Veterinary Care and SOP VC-003 (Rounds Observations). All animals were evaluated twice daily with reference to SOP VC-016 (Recognizing Pain, Stress and/or Distress). Clinical laboratory diagnostic procedures were performed as needed. Veterinary care was given as appropriate to each individual animal in accordance with the Program of Veterinary Care.

### Blood Analysis

Blood was analyzed for white blood cell count, red blood cell count, hemoglobin, hematocrit, MCV, MCHC, MCH, and platelet count along with a complete differential. In addition, a 22-test chemistry screen was performed consisting of Glucose, Urea Nitrogen, Creatinine, Total Protein, Albumin, Total Bilirubin, Alkaline Phosphatase, ALT, AST, CPK, Cholesterol, Calcium, Phosphorus, Sodium, Potassium, Chloride, A/G Ratio, BUN/Creatinine Ratio, Globulin, Triglycerides, GGTP and Magnesium. Measurements were taken prior to the start of the study and then weekly during the course of the study.

### PK Analysis

Analysis of the blood level values and pharmacokinetics of the test article were performed as described in Gamble et al. (2018) Front Vet Sci. 165:1-9.

### Results

### Body Weight

The mean average weight change for dogs during the 12 weeks of the study was 0.04 kg (-0.43%).

### Food Consumption

The mean daily food consumption per week for dogs during the study was 204 g.

### Test Article Consumption

Five of the eight dogs had 100% acceptance of the chews. Three dogs had to be dosed on occasion during the study: Dog ID #13644 (dosed 6.5% of the time), Dog ID #13513 (dosed 2.4% of the time) and Dog ID #2784123 (dosed 17.3% of the time).

### Hematology and Serum Chemistry

Beginning in Week 1, there was a slight increase mean alkaline phosphatase (ALP) value for the group. This value remained stable until Week 7 when the group mean ALP value became increasingly elevated. The highest group mean value was observed during the final week of the study, but did not exceed the normal reference range. The cause of the group mean value elevations appeared to be due to three dogs (Dog ID #s 13536, 2753822 and 2808987). By the end of the study Dog ID #s 13536 and 2753822 were above 100 U/L, but did not exceed the normal high of 131 U/L. Thus, their levels remained within the normal reference range. The observed elevations in only a few animals in the group may indicate individual sensitivity to the product. All other blood parameters remained within normal limits and no apparent trends were noted.

### Clinical Observations

During the study, occasional instances of loose stool and emesis were recorded. Dog ID #13536 was observed having five instances of food or bile emesis and six instances of loose stool. Dog ID #13513 was observed having two instances of loose stool. Dog ID #27583822 was observed having two instances of food emesis and eight instances of loose stool. Dog ID #13644 was observed having 12 instances of loose stool. Dog ID #13490 was observed having two instances of loose stool. Dog ID #2808987 was observed having four instances of loose stool. Dog ID #2963028 was observed having six instances of loose stool. Dog ID #2784123 was observed having six instances of loose stool. Occasional episodes of loose stool and bile emesis are not unusual in the dog colony and were not considered to be related to the test article. Clinical observations are listed in Table 7.

TABLE 7
Clinical observations

| Dos ID | Date | Observation | Dag ID | Date | Observation |
|---|---|---|---|---|---|
| 13490 | Jan. 18, 2018 | Loose stool | 2753822 | Jan. 18, 2018 | Loose stool |
| 1349ft | Feb. 4, 2018 | Loose stool | 2753822 | Jan. 2, 2018 | Food vomit |
| 13513 | Jan. 16, 2018 | Small amount loose stool | 2753822 | Feb. 10, 2018 | Loose stool |
| 33513 | Jan. 18, 2018 | Loose stool | 2753822 | Mar. 15, 2018 | Loose stool with blood |
| 13513 | Jan. 19, 2018 | Afraid and shaking head | 2753822 | Mar. 18, 2018 | Loose stool |
| 13513 | Jan. 25, 2018 | Shaking head | 2253822 | Mar. 20, 2018 | Loose stool |
| 13534 | Jan. 14, 2018 | Food and chew vomit | 2253822 | Mar. 24, 2018 | Two instances of loose stool |
| 13536 | Jan. 18, 2018 | Loose stool | 2753822 | Mar. 29, 2018 | Loose stool |
| 13536 | Jan. 22, 2018 | Food vomit prior to dosing | 2753822 | Mar. 31, 2018 | Food vomit |
| 13534 | Jan. 26, 2018 | Food vomit prior to dosing | 2784123 | Jan. 18, 2018 | Loose stool |
| 13536 | Jan. 28, 2018 | Bile vomit | 2784123 | Mar. 18, 2018 | Loose stool |
| 13536 | Feb. 4, 2018 | Bile vomit with blood | 2784123 | Mar. 21, 2018 | Loose stool |
| 13536 | Feb. 12, 2018 | Loose stool with mueus | 2784123 | Mar. 24, 2018 | Two instances of loose stool |
| 13536 | Feb. 15, 2018 | Loose stool | 2784123 | Mar. 29, 2018 | Loose stool |
| 13536 | Feb. 16, 2018 | Loose stool | 2808987 | Jan. 18, 2018 | Loose stool |
| 13536 | Mar. 21, 2018 | Loose stool | 2808987 | Feb. 5, 2018 | Loose stool |
| 13536 | Mar. 24, 2018 | Loose stool | 2808987 | Feb. 10, 2018 | Loose stool |
| 13644 | Jan. 18, 2018 | Loose stool | 2803987 | Mar. 20, 2018 | Loose stool |
| 13644 | Feb. 2, 2018 | Loose stool with mucus | 2963025 | Jan. 18, 2018 | Loose stool |
| 13644 | Feb. 4, 2018 | Loose stool | 2943028 | Feb. 10, 2018 | Loose stool |
| 13644 | Feb. 5, 2018 | Loose stool with mucus | 2963029 | Mar. 20, 2018 | Loose stool |
| 13644 | Feb. 6, 2018 | Loose stool | 2963025 | Mar. 22, 2018 | Loose stool |
| 13644 | Feb. 7, 2018 | Loose stool | 2963028 | Mar. 24, 2018 | Loose stool |
| 13648 | Feb. 10, 2018 | Loose stool | 2963028 | Mar. 25, 2018 | Loose stool |
| 13644 | Feb. 11, 2018 | Loose stool | | | |
| 13644 | Feb. 15, 2018 | Loose stool | | | |
| 13644 | Mar. 15, 2018 | Loose stool | | | |
| 13644 | Mar. 17, 2018 | Loose stool | | | |
| 13644 | Mar. 20, 2018 | Loose stool with mucus | | | |

Conclusions There were no adverse effects on body weights or food consumption. Group mean alkaline phosphatase values exhibited mild elevations during the study without exceeding the normal reference range. The remaining hematology and serum chemistry results remained within normal limits throughout the study and apparent trends were not observed over time. No clinical observations that were considered to be related to the administration of the test article were observed for any of the dogs during the course of the study. Overall acceptance of the treat was 96.7% with 5 out of 8 consuming the treat 100% of the time for the duration of the study.

### Example 13: Canine Pilot Study

A pilot study was conducted to assess the effectiveness of ElleVet Mobility Oil on the treatment of osteoarthritis in canines.

### Methods

Five dogs suffering from end stage osteoarthritis, joint pain, and geriatric pain were selected for the study, as shown in Tables 8 and 9.

Per manufacturer's instructions, dogs were given a loading dose of 2 mg/kg every 12 hours for the first 2 weeks then reduced to 1mg/kg every 12 hours for 2 weeks. Dogs were then returned to doses of 2 mg/kg every 12 hours for the final four weeks of the study.

On days 0, 14, 30, and 60, dogs were evaluated by flexion and extension measurements, muscle musculature measurements, a canine brief pain inventory survey, and a gait analysis using a pressure sensing walkway.

TABLE 8
Animal Information

| | | | | | | OA | Body | |
|---|---|---|---|---|---|---|---|---|
| Patient | | | | | Weight | Score | Condition | |
| Number | Name | Age | Sex | Breed | (#) | (0-3) | (1-9) | Medications |
| 3496 | Gipper | 12 yrs | FS | Golden | 64.9 | R: 3 | 6 | Rimadyl, apoquel, |
| | Hatch | 6 mo | | Retriever | | L: 2 | | dasuquin advanced |
| 21652 | Rocoo | 15 yrs | MN | Mixed | 67.7 | B: 2 | 5 | Keppra, Galliprant |
| | Payne | 4 mo | | Breed | | | | |
| 13750 | Bubba | 14 yrs | MN | Labrador | 65.6 | R: 2 | 4 | Galliprant, |
| | Schlimm | 8 mo | | Retriever | | L: 1 | | Gabapentin, |
| | | | | | | | | Theophyline |
| 24478 | Aiden | 7 yrs | MN | German | 86 | B: 2-3 | 5-5 | Gabapentin, rimadyl |
| | Langhans- | 2 mo | | Shepherd | | | | as needed |
| | Lindstadt | | | | | | | |
| 19821 | Moose | 11 yrs | MN | Mixed | 65 | L: 3/3 | 5 | Tramadol as needed |
| | Baker | 7 mo | | Breed | | | | |

TABLE 9
Animal history

| Patient | | | |
|---|---|---|---|
| Number | Name | Enrollment Date | History Notes |
| 3496 | Gipper | Wed, Jun. 11, 2014 | Bilateral medial shoulder syndrome (Subscapular tendinopathy); |
| | Hatch | | Bilateral chronic supraspinatus insertionopathy—Bilateral shoulder |
| | | | arthroscopy and radio-frequency treatment; Hobbles application, |
| | | | Bilateral elbow arthroscopy (2011); ADPC/PRP injections—bilateral |
| | | | supraspinatus, Intra-articular injections ADPC/ACS—bilateral |
| | | | shoulders (2011,2012); ADPC/PRP injection—Bilateral biceps, Left |
| | | | teres (2014); ADPC/PRP injection—right shoulder, elbow, biceps |
| | | | (2016); PRP injection - right elbow (2016); OsteoBioScaff injection— |
| | | | right elbow (2017). |
| 21652 | Rocoo | Fri, Jun. 13, 2014 | Elbow arthritis, history of seizure activity, history of elevated liver |
| | Payne | | enzymes |
| 13750 | Bubba | Tue, Jun. 17, 2014 | Bilateral elbow OA, hind limb weakness |
| | Schlimm | | |
| 24478 | Aiden | Tue, Jul. 1,2014 | Bilateral Hip Dysplasia |
| | Langhans- | | |
| | Lindstadt | | |
| 19821 | Moose | Fri, Jul. 18, 2014 | Left medial shoulder syndrome, bilateral surpaspinatus |
| | Baker | | tendinopathies (R > L), L FCP -> L elbow scope & L RF tx performed |
| | | | (May 2016); L elbow OA |

### Results

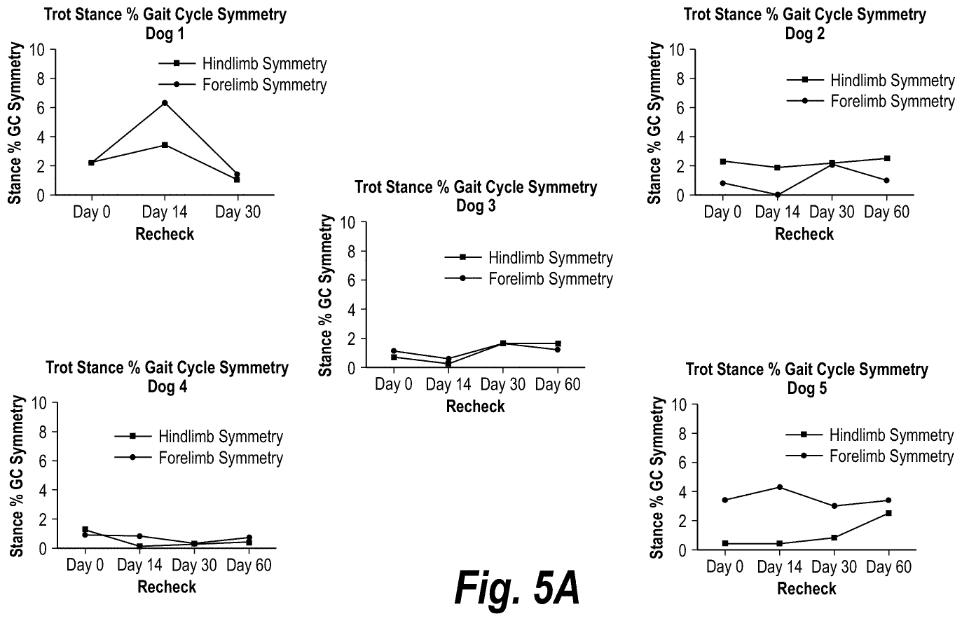

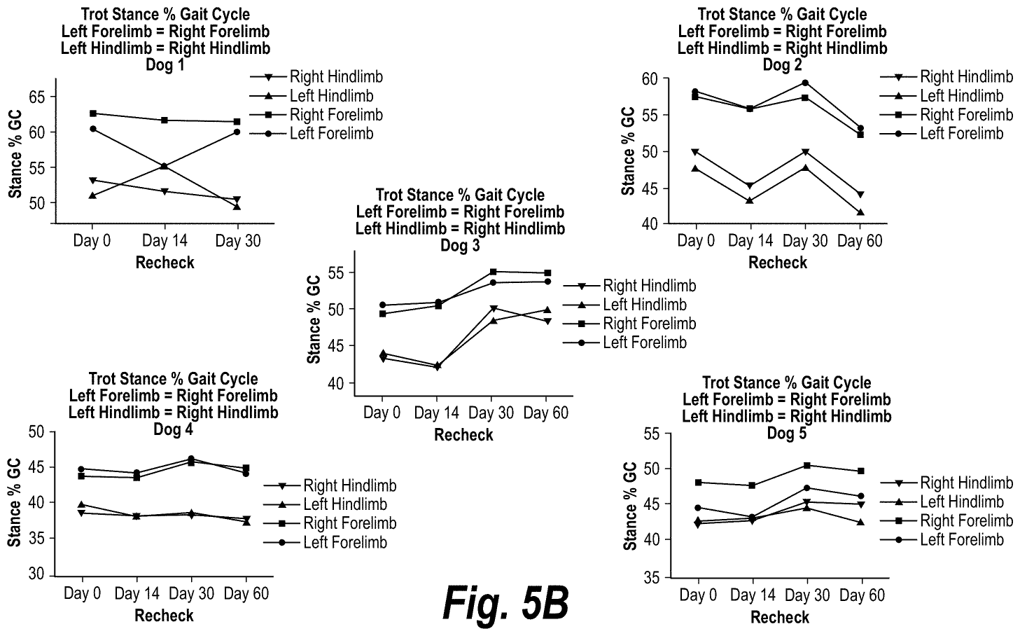

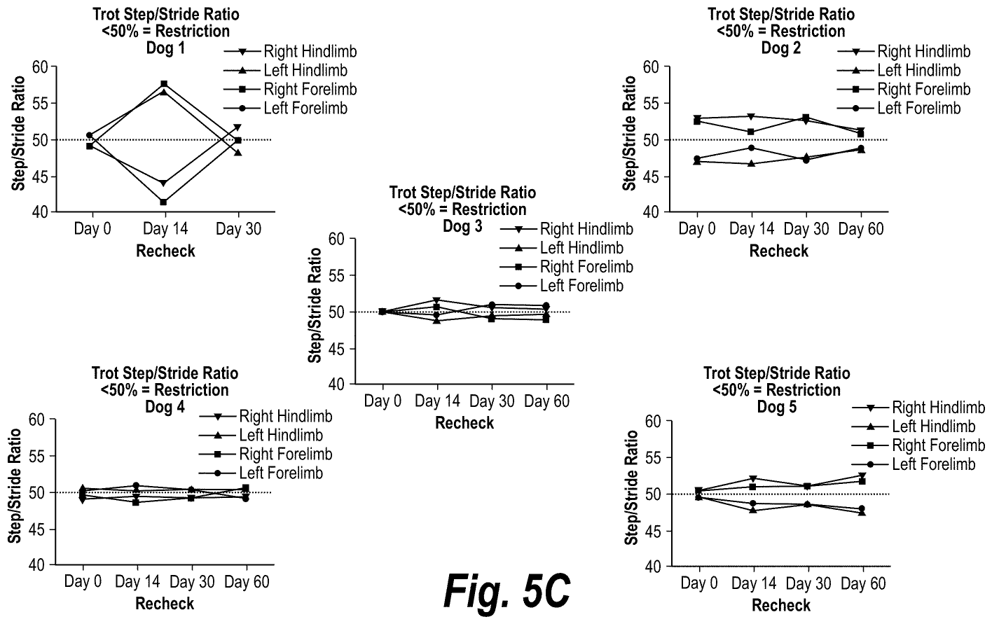

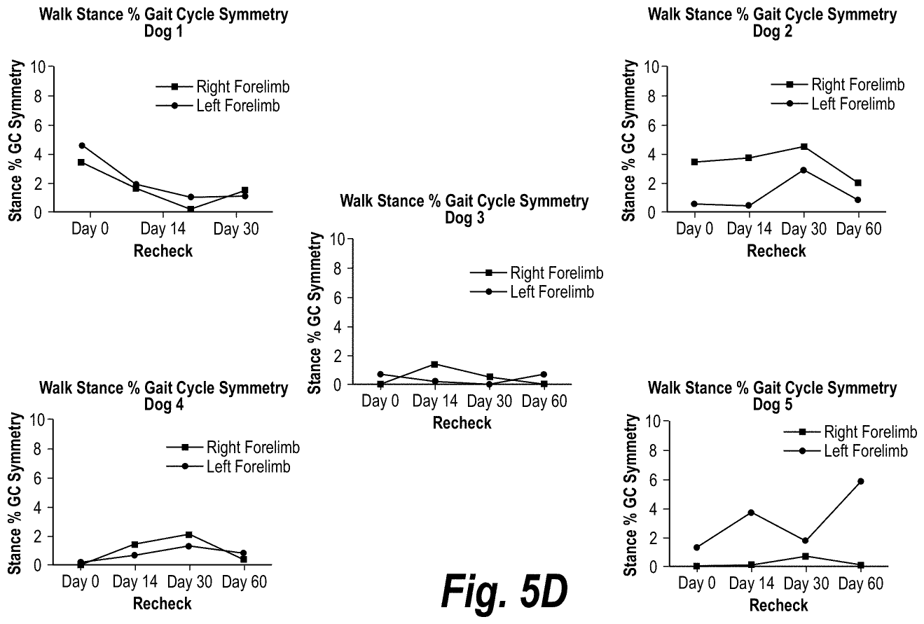

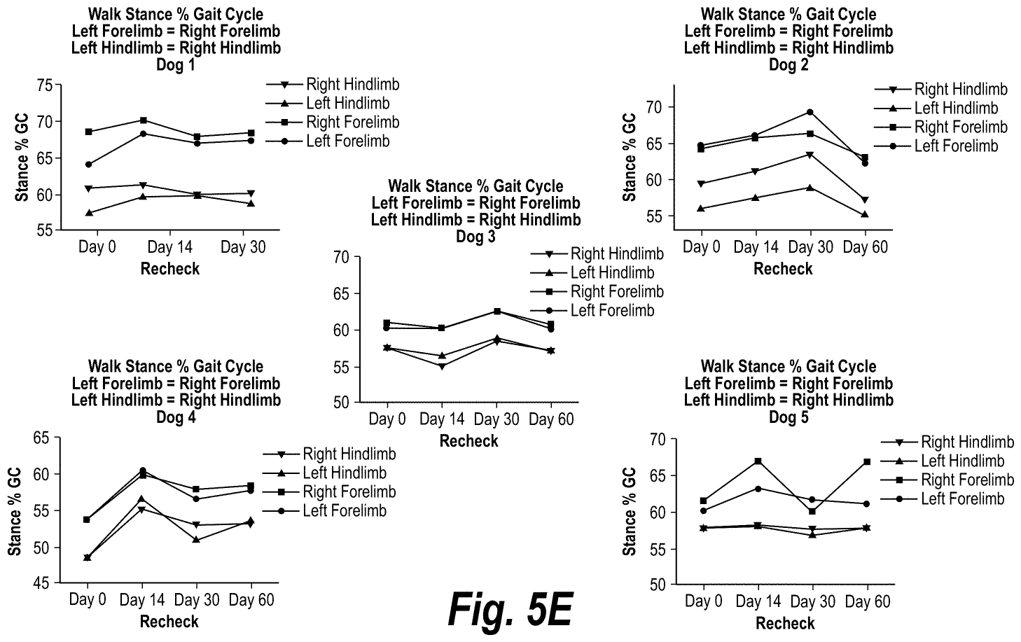

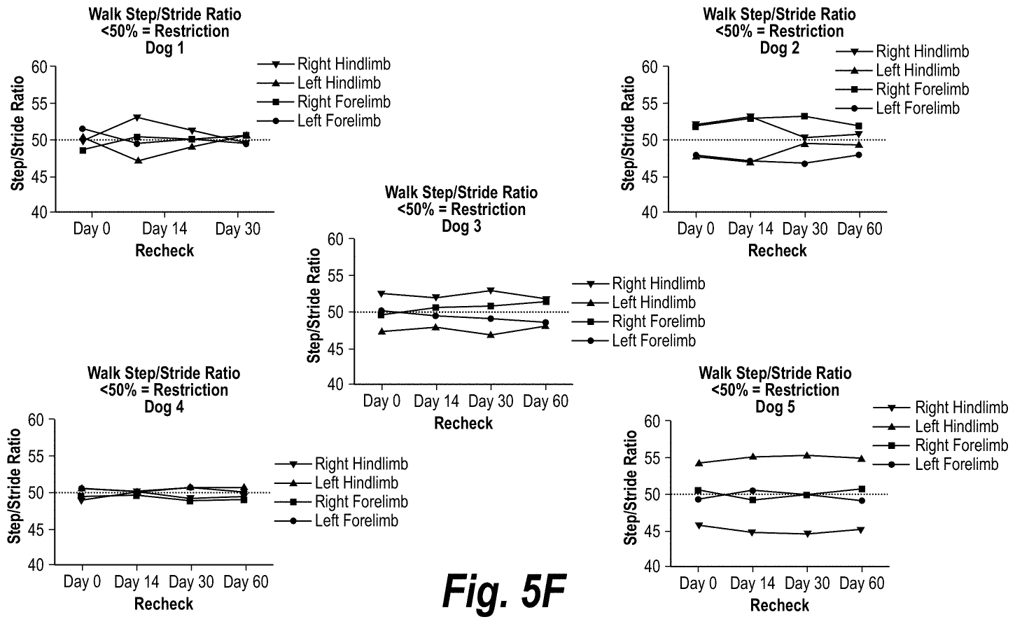

Three out of five owners (60%) reported a significant improvement in pain severity score and pain interference score. Gait analysis revealed that total pressure index (TPI%), step/stride ratio, and stance percentage did not significantly improve or decline throughout the length of the study, as shown in FIGS. 5A-5F. Flexion improved in 3 out of 5 dogs and declined by >5 degrees in 2 out of 5 dogs. Extension improved in 2 out of 5 dogs and declined in 1 out of 5 dogs. Following completion of the study, 3 out of 4 owners that respond to a questionnaire indicated that they would like to continue using the supplements. Improvements observed by owners included improved function and comfort laying down, rising, resting, walking, energy, playing, and overall health.

### Example 14: Feline Safety Study

A 12-week safety study was performed in felines to evaluate the safety of an oil containing CBD.

### Animals and Study Design

Eight cats, 2-6 years old, weighing 3.33-5.17 kg at study start were selected for the study, as shown in Table 10.

TABLE 10
Animal information

| Cat ID | Sex | Date of Birth |
|---|---|---|
| 15EGA5 | FS | May 8, 2015 |
| 13IRD3 | FS | Nov. 5, 2013 |
| 15KGA2 | FS | Apr. 7, 2015 |
| 13CNL3 | MC | Sep. 20, 2013 |
| 13CCL1 | MC | Feb. 11, 2013 |
| GJY3 | MC | Jul. 17, 2011 |
| 15KGC3 | MC | Apr. 8, 2015 |
| 13CP37 | FS | Nov. 25, 2013 |

Cats were single housed in cages of a size in accordance with the Animal Welfare Act, with a 12-hour-light/12-hour-dark cycle and targeted conditions of 50° to 85° F. Cages, food bowls, water bowls and litter boxes were cleaned daily and sanitized in accordance with the Animal Welfare Act. Fresh tap water, fit for human consumption, was available ad libitum by means of stainless steel bowls. There were no known contaminants that were reasonably expected to be present in the dietary material that were known to be capable of interfering with the purpose or conduct of the study

During the study, the control diet, Purina Cat Chow, was the sole source of food supplied to each animal once daily for approximately 4 hours. Cats were fed according to ideal body condition and were fasted for a minimum of 12 hours prior to blood collections. CBD oil was orally administered twice a day using a 1 ml syringe at a dosage of 2 mg/kg. The total dose per 24 hour period was 4 mg/kg. Dosing is shown in Tables 11 and 12.

TABLE 11
Dosage per week (mL) (weeks 1-6)

| | | Week | | | | | |
|---|---|---|---|---|---|---|---|
| Dog ID | Sex | 1 | 2 | 3 | 4 | 5 | 6 |
| I5EGA5 | FS | 0.14 | 0.14 | 0.14 | 0.15 | 0.14 | 0.14 |
| 13IRD3 | FS | 0.13 | 0.14 | 0.14 | 0.14 | 0.14 | 0.14 |
| 15KGA2 | FS | 0.13 | 0.14 | 0.14 | 0.14 | 0.14 | 0.14 |
| 13CNL3 | MC | 0.19 | 0.19 | 0.19 | 0.19 | 0.19 | 0.19 |
| 13CCL1 | MC | 0.20 | 0.20 | 0.20 | 0.20 | 0.21 | 0.20 |
| GJY3 | MC | 0.21 | 0.22 | 0.22 | 0.22 | 0.23 | 0.23 |
| 15KGC3 | MC | 0.19 | 0.20 | 0.21 | 0.21 | 0.21 | 0.21 |
| 13CPJ7 | FS | 0.15 | 0.15 | 0.15 | 0.15 | 0.16 | 0.16 |

TABLE 12
Dosage per week (mL) (weeks 7-12)

| | | Week | | | | | |
|---|---|---|---|---|---|---|---|
| Dog ID | Sex | 7 | 8 | 9 | 10 | 11 | 12 |
| 15EGA5 | FS | 0.14 | 0.14 | 0.14 | 0.14 | 0.14 | 0.13 |
| 13IRD3 | FS | 0.14 | 0.14 | 0.14 | 0.14 | 0.13 | 0.13 |
| 15KGA2 | FS | 0.14 | 0.14 | 0.14 | 0.14 | 0.14 | 0.13 |
| 13CNL3 | MC | 0.19 | 0.19 | 0.19 | 0.19 | 0.19 | 0.19 |
| 13CCL1 | MC | 0.21 | 0.20 | 0.20 | 0.20 | 0.19 | 0.19 |
| GJY3 | MC | 0.23 | 0.23 | 0.23 | 0.23 | 0.23 | 0.22 |
| 15KGC3 | MC | 0.21 | 0.21 | 0.21 | 0.21 | 0.21 | 0.21 |
| 13CP17 | FS | 0.16 | 0.15 | 0.15 | 0.15 | 0.15 | 0.15 |

### CBC and Serum Chemistry

Prior to study initiation, 5 milliliters of blood was collected for each cat and was used to determine eligibility for the study. During the study, 5 milliliters of blood was collected weekly (±2 days). Blood was collected via jugular venipuncture in sterile syringes. Samples were split into two tubes: a red-top serum separator tube and a lavender-top EDTA tube. Redtop tubes were spun in a refrigerated centrifuge for 15 minutes at 3000 RPM after being allowed to clot. Lavender-top tubes were placed on a rocker to allow the blood to adequately mix with the anticoagulant. Blood samples were packaged and sent by priority-overnight to Antech Diagnostics for analysis.

### Pharmacokinetic (PK) Blood Collection

On the first day of dosing, blood was collected for a PK analysis from 6 of the 8 cats. The most cooperative cats were selected for the PK analysis. Approximately 4 milliliters of blood was collected via jugular venipuncture in sterile syringes at one day prior to treatment (timepoint 0) and then 1, 4, 8 and 24 hours after treatment. Samples were placed into a red top clotting tube with no serum separator. Serum was harvested by centrifuging the tubes at 3000 RPM for 15 minutes. The harvested serum was placed in cyrovials stored at −70° C. Each tube was labeled with the cat id, date of collection and collection time point. Samples were shipped overnight on dry ice to the Proteomics & Metabolomics Facility at Colorado State University.

Clinical Observations A veterinarian performed a complete physical examination to all cats prior to the initiation of the study and at study completion. Each cat was evaluated as to general health, body and hair coat condition. Qualified personnel performed clinical observations twice daily in accordance with Summit Ridge Farms' Program of Veterinary Care and SOP VC-003 (Rounds Observations). All animals were evaluated twice daily with reference to SOP VC-016 (Recognizing Pain, Stress and/or Distress). Clinical laboratory diagnostic procedures were performed as needed. Veterinary care was given as appropriate to each individual animal in accordance with the Program of Veterinary Care.

### Blood Analysis

Blood was analyzed for white blood cell count, red blood cell count, hemoglobin, hematocrit, MCV, MCHC, MCH, and platelet count along with a complete differential. In addition, a 22-test chemistry screen was performed consisting of Glucose, Urea Nitrogen, Creatinine, Total Protein, Albumin, Total Bilirubin, Alkaline Phosphatase, ALT, AST, CPK, Cholesterol, Calcium, Phosphorus, Sodium, Potassium, Chloride, A/G Ratio, BUN/Creatinine Ratio, Globulin, Triglycerides, GGTP and Magnesium. Measurements were taken prior to the start of the study and then weekly during the course of the study.

### PK Analysis

Extraction of Cannabidiol from Feline serum for LC-MS

Aliquots of feline serum were delivered to the facility on dry ice and stored at −80° C. upon receipt. For cannabidiol (CBD) extraction, serum was thawed on ice and 50 pL of each sample was placed into a 2.0 ml glass extraction vial (VWR ROBO Unassembled Autosampler Vial) kept on chilled on ice. 200 pL of cold (−20C.) 100% Acetonitrile (spiked with 60 ng/mL of d3-CBD) was added to each sample and vortexed at room temperature for 5 minutes. 200 μL of water was added and vortexed for an additional 5 minutes. 1 ml of 100% hexane was then added to each sample and vortexed for a final 5 minutes. Phase separation was enhanced under centrifugation at 3000 rpm for 15 minutes at 4C. The upper hexane layer was transferred to new-labeled glass vials (˜900 uL per sample), carefully avoiding the middle and lower layers. Samples were concentrated to dryness under N₂ and resuspended in 60 μL of 100% acetonitrile (Zgair et al. (2015) J Pharm Biomed Anal. 114:145-51).

### Standard Curve

An 8 point standard curve of CBD was generated in matrix background using a blank serum. Concentrations ranged from 0 ng/mL 1000 ng/mL (3.2X dilution series). 50 uL of each spiked serum sample was extracted as above.

### LC-MS/MS Analysis

LC-MS/MS was performed on a Waters Acquity M-Class UPLC coupled to a Waters Xevo TQ-S triple quadrupole mass spectrometer. Chromatographic separations were carried out on a Waters BEH C18 iKey Separation Device (150 μm ×50 mm, 1.7 μM). Mobile phases were 99.9% acetonitrile, 0.1% formic acid (B) and water with 0.1% formic acid (A). The analytical gradient was as follows: time=0 min, 70% B; time=1.0 min, 70% B; time=6 min, 100% B; time 7.0 min, 100% B; time 7.5 min, 70% B. Total run time was 10 minutes. Flow rate was 3.0 μL/min and injection volume was 2.0 μL. Samples were held at 6° C. in the autosampler, and the column was operated at 70° C. The MS was operated in selected reaction monitoring (SRM) mode, where a parent ion is selected by the first quadrupole, fragmented in the collision cell, then a fragment ion selected for by the third quadrupole. Product ions, collision energies, and cone voltages were optimized for each analyte by direct injection of individual synthetic standards. Inter-channel delay was set to 3 ms. The MS was operated in positive ionization mode with the capillary voltage set to 3.6 kV. Source temperature was 120° C. and desolvation temperature 992° C. Desolvation gas flow was 1 L/hr, cone gas flow was 150 L/hr, and collision gas flow was 0.2 mL/min. Nebulizer pressure (nitrogen) was set to 7 Bar. Argon was used as the collision gas.

### Data Analysis and Statistics

All Raw data files were imported into the Skyline open source software package (MacLean et al. (2010) Bioinformatics. 26(7):966-8). Each target analyte was visually inspected for retention time and peak area integration. Peak areas were extracted for target compounds detected in biological samples and normalized to the peak area of the appropriate internal standard in each sample using in-house R Script (TQS-tools). CBD concentrations were calculated in nanograms per milliliter of extract (0.06 mL) and then back calculated to nanograms per mL of serum (0.05 mL of serum).

Calculation of Variance using QC Pool

50 uL of all serum samples (feline and canine) were pooled into a single Quality Control sample and 50 uL was extracted as described above. The QC pool was injected every 10 samples and CBD concentrations were used to measure the technical variance over the course of data acquisition.

### Limits of Detection (LOD) and Limits of Quantification (LOQ)

The LOD and LOQ represent the lower limits of detection and quantification for each compound in the matrix of this study. LOD are calculated based on the standard deviation of the response (Sy) of the 0 point calibration standard (i.e. 0 ng/mL CBD as an estimate on noise) and the slope of the calibration curve (S) at levels approximating the LOD according to the formula: LOD=3*(Sy/S). LOQ=10*(Sy/S). The Sy of y is the standard deviation used for LOD and LOQ calculation (Shrivastava (2011) Chronicles of Young Scientists. 2:21-5; Broccardo et al. (2013) Chromatogr B Analyt Technol Biomed Life Sci. 934:16-21).

### Results

### Body Weight

The mean average weight change for cats during the 12 weeks of the study was 0.06 kg (1.04%).

### Food Consumption

The mean daily food consumption per week for cats during the study was 62 g.

### Test Article Acceptance

Overall all cats exhibited behaviors of licking, salivating, pacing, head shaking, chomping, dose resentment (uncooperative behavior), etc. at various intervals throughout the study that were indicative of dislike of the test article.

### Hematology and Serum Chemistry

Beginning in Week 2, there was an increase in the mean alanine aminotransferase (ALT) value for the group. This value remained increased from baseline until the end of the study. Mild increases in individual ALT levels were observed in the majority of the cats throughout the study. The cat with the greatest increase in ALT (above the normal reference range of 100 U/L), with a concurrent increase in aspartate aminotransferase (AST), was Cat ID #13CNL3. Beginning in Week 4, this cat's ALT and AST levels began to decrease, but remained elevated from baseline. ALT levels remained above the normal reference range, shown in Table 70, for the duration of the study. Also during Week 2, the ALT levels of Cat ID #s 131RD3 and 13CPJ7 increased by 23 to 31 U/L, respectively, from baseline values. The ALT levels of Cat ID #13CPJ7 returned to baseline by Week 10. At Week 4, the ALT of Cat ID #13CCL1 was elevated from baseline by 32 U/L. Levels returned to baseline by Week 10. The test article appeared to cause mild ALT changes in the majority of cats with one cat maintaining elevated ALT levels above normal limits throughout the study. The group mean values of all other blood parameters remained within normal limits and no apparent trends were noted.

### Clinical Observations

During the study, occasional instances of loose stool and emesis were recorded, as shown in Table 13. Cat ID #13CCL1 was observed having five instances of food emesis. Cat ID #13CNL3 was observed having one instance of hairball emesis and one instance of hair and bile emesis. Cat ID #131RD3 was observed having one instance of food emesis. Cat ID #15EGA5 was observed having three instances of food vomit and one instance of hair and bile emesis. Cat ID #GJY3 was observed having two instances of hairball emesis and one instance of food emesis. Occasional episodes of hairball and food emesis are not unusual in the cat colony and were not considered to be related to the test article.

TABLE 13
Clinical observations

| Cat Id | Date | Observation |
|---|---|---|
| 13CCLI | Jan. 19, 2018 | Very calm, relaxed prior to dosing in am |
| | | and pm |
| 13CCLI | Jan. 21, 2018 | Very calm, relaxed prior to dosing in pm |
| 13CCLI | Jan. 25, 2018 | Food vomit |
| 13CCLI | Jan. 31, 2018 | Food vomit |
| 13CCLI | Feb. 9, 2018 | Food vomit |
| 13CCLI | Mar. 2, 2018 | Food vomit |
| 13CCLI | Apr. 8, 2018 | Food vomit |
| 13CNL3 | Jan. 19, 2018 | Very calm, relaxed prior to dosing in am |
| | | and pm |
| 13CNL3 | Jan. 21, 2018 | Very calm, relaxed prior to dosing in pm |
| 13CNL3 | Jan. 22, 2018 | Very relaxed |
| 13CNL3 | Feb. 4, 2018 | Hairball vomit |
| 13CNL3 | Apr. 6, 2018 | Bile vomit and hairball vomit |
| 13IRD3 | Feb. 8, 2018 | Serai digested food vomit |
| 15EGAS | Jan. 26, 2018 | Food vomit |
| 15EGA5 | Feb. 8, 2018 | Semi digested food vomit |
| 15EGA5 | Mar. 9, 2018 | Bile vomit and hairball vomit |
| 15EGAS | Mar. 19, 2018 | Digested food vomit |
| 15KGA2 | Jan. 19, 2018 | Very calm, relaxed prior to dosing in am |
| | | and pm |
| 15KGA2 | Jan. 21, 2018 | Very calm, relaxed prior to dosing in am |
| | | and pm |
| 15KGA2 | Jan. 12, 2018 | Very relaxed |
| 15KGC3 | Jan. 19, 2018 | Very calm, relaxed prior to dosing in pm |
| GJY3 | Jan. 31, 2018 | Hairball vomit |
| GJY3 | Feb. 18, 2018 | Digested food vomit |
| GJY3 | Mar. 19, 2018 | Hairball vomit |

### PK Data

Table 14 shows the quantification of cannabidiol in feline serum and Table 15 shows cat cannabadiol pharmacokinetics.

TABLE 14
Cannabidiol quantification in feline serum.

| PMF | | | | Replicate | ppb in |
|---|---|---|---|---|---|
| No. | Animal ID | Species | Time Point | (A or B) | Serum |
| 53 | 13CCL1 | Feline | 1 day prior | A | ND |
| 53 | | | 1 day prior | B | ND |
| 59 | | | 60 min | B | 32.85 |
| 59 | | | 60 min | A | 34.26 |
| 65 | | | 4 hr | B | 1.69** |
| 65 | | | 4 hr | A | 1.82** |
| 71 | | | 8 hr | B | 65.42 |
| 71 | | | 8 hr | A | 79.30 |
| 77 | | | 24 hr | B | 42.76 |
| 77 | | | 24 hr | A | 44.88 |
| 52 | 13CNL3 | Feline | 1 day prior | A | ND |
| 52 | | | 1 day prior | B | ND |
| 58 | | | 60 min | B | 24.44 |
| 58 | | | 60 min | A | 26.32 |
| 64 | | | 4 hr | A | ND |
| 64 | | | 4 hr | B | ND |
| 70 | | | 8 hr | B | 1.82** |
| 70 | | | 8 hr | A | 2.22* |
| 76 | | | 24 hr | A | 141.92 |
| 76 | | | 24 hr | B | 147.74 |
| 50 | 13IRD3 | Feline | 1 day prior | A | ND |
| 50 | | | 1 day prior | B | ND |
| 56 | | | 60 min | B | 44.14 |
| 56 | | | 60 min | A | 45.40 |
| 62 | | | 4 hr | B | 1.53** |
| 62 | | | 4 hr | A | ND |
| 68 | | | 8 hr | A | ND |
| 68 | | | 8 hr | B | ND |
| 74 | | | 24 hr | A | 10.28 |
| 74 | | | 24 hr | B | 10.31 |
| 49 | 15EGA5 | Feline | 1 day prior | A | ND |
| 49 | | | 1 day prior | B | ND |
| 55 | | | 60 min | B | 28.10 |
| 55 | | | 60 min | A | 31.02 |
| 61 | | | 4 hr | A | ND |
| 61 | | | 4 hr | B | ND |
| 67 | | | 8 hr | A | 44.23 |
| 67 | | | 8 hr | B | 46.05 |
| 73 | | | 24 hr | A | 13.95 |
| 73 | | | 24 hr | B | 17.17 |
| 51 | 15KGA2 | Feline | 1 day prior | A | ND |
| 51 | | | 1 day prior | B | ND |
| 57 | | | 60 min | A | ND |
| 57 | | | 60 min | B | ND |
| 63 | | | 4 hr | A | ND |
| 63 | | | 4 hr | B | ND |
| 69 | | | 8 hr | B | 365.18 |
| 69 | | | 8 hr | A | 376.28 |
| 75 | | | 24 hr | B | 0.18** |
| 75 | | | 24 hr | A | 0.36** |
| 54 | GJY3 | Feline | 1 day prior | A | ND |
| 54 | | | 1 day prior | B | ND |
| 60 | | | 60 min | A | 378.59 |
| 60 | | | 60 min | B | 535.08 |
| 66 | | | 4 hr | A | 51.48 |
| 66 | | | 4 hr | B | 68.31 |
| 72 | | | 8 hr | A | 71.64 |
| 72 | | | 8 hr | B | 79.59 |
| 78 | | | 24 hr | B | 33.12 |
| 78 | | | 24 hr | A | 35.88 |

Cannabidiol quantification in Feline Serum is report ed as ng/mL (ppb).
ND = Not Detected (no quantifiable value).
* = values below calculated Limit of Quantification (6.2 ppb).
** = values below calculated Limit of Detection (1.9 ppb).

TABLE 15
Cat cannabadiol pharmacokinetics

| Cat # | Cmax | Tmax | T1/2 el | AUC 0 -> t | MRT |
|---|---|---|---|---|---|
| 15EGA5 | 75.3 | 1 | 1.2 | 212.2 | 2.1 |
| 13IRD3 | 40.5 | 1 | 1.3 | 125.0 | 2.4 |
| 15KGA2 | 53.3 | 1 | 1.7 | 154.1 | 2.9 |
| 13CNL3 | 21.2 | 4 | 1.7 | 134.2 | 5.4 |
| 13CCL1 | 20.4 | 1 | 1.7 | 60.2 | 2.7 |
| GIY3 | 47.6 | 4 | 1.2 | 265.0 | 5.7 |
| 15KGC3 | 8.8 | 1 | 2.3 | 54.2 | 3.8 |
| 13CP17 | 12.1 | 1. | 2.3 | 42.4 | 2.4 |

Oral administration of 2/mg/kg cannabidiol in capsule form
Cmax = Maximum concentration (ng/mi)
Tmax = Time of maximum concentrstion (hr)
T1/2 el = Half-life of elimination (hr)
AUC 0-t = Area under the curve (0 time to time of last collection [24 hr]) (ng-hr/ml)
MRT = Mean residence time (hr)

The LOD for CBD in feline serum was calculated to be 1.9 ng/mL (ppb in serum). The LOQ for CBD in feline serum was calculated to be 6.2 ng/mL (ppb in serum).

### Conclusions

There were no adverse effects on body weights or food consumption. Group mean alanine aminotransferase values exhibited elevations during the study that peaked at Week 2. Levels decreased during the following weeks, but did not return to baseline levels. ALT levels of one cat (Cat ID #13CNL3) remained significantly elevated throughout the study, exceeding normal reference ranges for the duration of the treatment period. The remaining group mean hematology and serum chemistry values remained within normal reference limits throughout the study and apparent trends were not observed over time. No adverse clinical observations that were considered to be related to the administration of the test article were observed for any of the cats during the course of the study. However, acceptance of the test article was considered to be poor.

### Example 15: Canine Oncology Safety Study

A study is conducted to 1) assess the safety and tolerability of oral CBD administration to dogs undergoing L-CHOP chemotherapy for the treatment of high-grade lymphoma, 2) describe health related quality of life (HRQL) assessment differences between patients receiving an oral CBD oil versus placebo in addition to L-CHOP/CHOP chemotherapy, and 3) assess doxorubicin peak concentrations and elimination kinetics after chemotherapy (week 9/10).

### Animals and Study Design

All canine patients have a cytologic or histologic diagnosis of intermediate to high-grade multicentric lymphoma and undergo treatment with L-CHOP (week 5-11) chemotherapy or CHOP (week 4-10) chemotherapy. The patients are of a body weight >15 kg, (to allow for use of standard doses of chemotherapy) and are entering the end of the first cycle of L-CHOP chemotherapy (first doxorubicin treatment).

Other exclusion criteria are defined as: patient is not expected to survive at least 10 weeks from the day of initiation of L-CHOP treatment, has significant co-morbidities that would interfere with the ability to assess their response to treatment, has documented liver dysfunction (defined as total bilirubin greater than 1.5× the high end of the reference interval). Breeds at high-risk for the Multi-Drug Resistant (MDR1) mutation are not included unless they prove that they do not carry a mutation in this gene.

This study is approved by the UF Institutional Animal Care and Use Committee (IACUC) and an informed signed owner consent is obtained for all patients on the day of enrollment.

Patients receiving chemotherapy for lymphoma are randomized to receive either placebo or CBD oil starting at week 4 or 5 of a doxorubicin based chemotherapy protocol and continue for 5 weeks. Patients are assessed with a physical examination, complete blood cell count (CBC), chemistry panel, and urinalysis (UA) prior to starting the study and are monitored as described Table 16.

TABLE 16
Patient assessment schedule

| Bolded x's supported by CT | Week 4/5 | Week 6/7 | Week 7/8 | Week 8/9 | Week 10/11 |
|---|---|---|---|---|---|
| Physical exam | x | x | x | x | x |
| CBC | x | x | x | x | x |
| Chemistry | x | | x | | x |
| UA | x | | x | | x |
| Chemotherapy | x | x | x | x | |
| Doxorubicin Elimination Testing | | | | x | |
| QOL survey | x | x | x | x | x |

Clinical information is collected at diagnosis, including signalment and clinical signs. Lymph nodes are measured at each visit by two experienced clinicians independently, including an oncology specialty intern, oncology resident, or a faculty member diplomate of the ACVIM in Oncology. Lymph node measurements are performed in accordance with the response evaluation published criteria for peripheral nodal lymphoma in dogs. Additional staging tests including thoracic radiographs, abdominal ultrasound, urinalysis, immunophenotyping and bone marrow aspirate are recommended for all dogs but are not required for enrollment. Dogs are staged as described by the World Health Organization's Clinical Staging System for Lymphoma in Domestic Animals. Clients are asked to fill out the QoL questionnaire, as described by Giuffrida et al., 2018, in addition to the typical information collected at each chemotherapy appointment (Giuffrida et al. (2018) J Amer Vet Med Assoc. 252:1073-1083.).

All dogs enrolled are randomized into groups receiving control (placebo) and treatment oils using the Randomizer Application. The pharmacy team provides the supplement (control or treatment) to allow for blinding of the clinicians. The dogs are supplied CBD to equate to 5 mg/kg body weight every 12 hours for the duration of the study. The placebo constitutes an equivalent volume of olive oil with 10 parts per thousands of anise oil and 5 parts per thousands of peppermint oil (to provide a similar herbal smell). The supplements are given with food every 12 hrs.

Supportive medications, including gastrointestinal protectants, antibiotics, anti-nausea medications, musculoskeletal supplements (i.e. glucosamine) and preventative flea/tick and heartworm medications, are permitted.

Owners are given enough treatment for each week of the clinical study and are asked to return their prior bottle to ensure it was given properly. Missed doses are recorded. All dogs have CBC, chemistry and UA performed on week 5, 8 and 10 to more completely assess safety during treatment. In addition, inflammation is evaluated at weeks 5, 7 and 10 with serum collection for C-reactive protein assessment.

Response to treatment at each visit is determined as outlined by the published response evaluation criteria for peripheral nodal lymphoma in dogs. A complete response (CR) is defined as a complete absence of identifiable abnormalities associated with lymphoma including normal lymph node size and consistency; a partial response (PR) is characterized by a decrease in the mean sum of the largest measured lymph node diameters of 30% or greater; stable disease (SD) is defined as a decrease in lymph node size of less than 30% or an increase of less than 20%; and progressive disease (PD) is an increase of greater than 20% of the mean lymph node diameter sum or unequivocal progression of lymphoma elsewhere in the body. Objective response rate is defined as the percentage of patients who achieved a complete or partial response to treatment. Toxicity is graded in accordance with the VCOG—common terminology criteria for adverse events one week after vinblastine administration.

### Doxorubicin Elimination Kinetics

Eight dogs from the treatment and the placebo groups undergo doxorubicin pharmacokinetic assessment. The assessment occurs at week 9/10 of doxorubicin treatment, after being on the CBD oil for 4 weeks, assuming that steady state concentrations are reached in the bloodstream. Dogs have blood drawn at the conclusion of their IV doxorubicin treatment to assess peak serum concentrations of doxorubicin. Then dogs have 2 cc of blood drawn at 5, 45 and 60 minutes post infusion to assess both elimination half time and doxorubicin exposure. These analyses are used to better understand if chronic CBD exposure alters chemotherapeutic pharmacokinetics (Wittenberg et al. (2014) Vet Comp Oncol. 12: 2: 114-119.).

### Statistical Analysis

The clinical data generated from this study provides ordinal data that allows for assessment of changes from baseline in each group over time. A power analysis reveals that for an average Likert score for a parameter of 2 to 1.5 (less severe disease), with a standard deviation of 0.7 within any specific score, 18 dogs are needed per group at a p value of 0.05 and desired power of 0.8. Therefore 20 dogs are enrolled in each arm. Data analysis occurs in the three categories of malaise, anxiety and gastrointestinal status using a generalized linear mixed model approach using fixed effects of time, treatment, time x treatment, gender, age, stage of disease and the random effects of dog.

Considering the numerical data generated from the doxorubicin elimination kinetics all of the data is handled using a two way analysis of variance examining time versus treatment with Tukey's post hoc analysis with an alpha set at 0.05.

### Example 16: Canine Epilepsy Study

A study is conducted to 1) determine the safety of hemp based nutraceuticals (HBNs) in dogs on anti-epileptic drugs (AED's), 2) assess the efficacy of adjunctive therapy with HBNs in reducing seizure frequency in dogs with epilepsy, and 3) assess AED serum levels and serum chemistry for organ function.

### Animals and Study Design

A recent study by Law and colleagues suggests that refractory seizures with medium chain triglycerides can ameliorate refractory seizures. Based on their methods, 24 dogs are enrolled in this study. All of these dogs have been diagnosed with refractory epilepsy and are assessed over a minimum of 3 months at an initial dose of 2 mg/kg of HBN (ELLEVET) every 12 hours. In addition, dogs receive their current AED regimen (phenobarbital, zonisamide, potassium Bromide, and/or leviteracetam) or an equal amount of placebo oil for a three month duration. After the initial 3 months of treatment, the dogs are crossed over to the second oil for another 3 months. Diagnosis of epilepsy is based on clinical findings, magnetic resonance imaging, cerebrospinal fluid analysis, and a prior three month seizure log (numbers and duration). Before enrollment, all dogs exhibit either generalized motor seizures or focal seizure episodes and are on optimized doses of maintenance AED at acceptable therapeutic serum levels.

For each dog, the number of seizures per week, the average duration of the seizures and the number of days that seizures occurred are compared for the 3 months before and the 3 months after starting treatment with HBNs or placebo oil. Comparisons are also made for another 3 months when crossed over to the second oil. A minimum 50 percent reduction in the number of seizures per week is interpreted as a positive clinical response to HBNs and is assessed statistically via Chi Square as responders and non-responders. Side effects are monitored closely (i.e., ataxia and sedation). At weeks 0, 2, 6, 12, 14, 18 and 24, all owners complete a brief seizure survey. In addition, owners keep a seizure diary/log throughout the entire 3 months regarding the frequency and duration of seizures during each arm of the trial. Blood is drawn for complete blood count, serum chemistry profile, AED and HBN levels prior to the initiation of HBNs and at 0, 2, 6, 12, 14, 18 and 24 weeks.

### Statistical Analysis

All CBC, chemistry, episodes per month, duration per seizure, AED change from baseline (numerical and quantitative) are evaluated using a mixed model analysis of variance. Analyses take fixed effects into account including weight, age, gender, neuter status, treatment, time and treatment crossed with time, and period. Time nested in period and dog within time and period are treated as random effects with differences over time and treatment evaluated using Tukey's post hoc analysis.

### Example 17: Canine Post-Operative Pain Relief Study

A study is conducted to 1) assess client specific outcomes regarding pain relief for 8 weeks post-tibial plateu leveling osteotomy surgery 2) use objective quantitative forcemat analysis at weeks 0, 2 and 8, and 3) assess serum chemistry for organ function and markers of chronic inflammation.

### Animals and Study Design

A population of 48 dogs is studied in an owner and veterinary placebo blinded study (24 placebo, 24 treatment). Prior to surgery, all dogs have a complete blood count and serum chemistry performed. All dogs receive routine pain control treatment with fentanyl immediately following surgery. Many dogs have undergone nerve blocks as well, which may be assessed in the statistical model. The treatment or placebo oil is administered in the evening on the day prior to surgery. The day following surgery, the dog is treated in the AM and PM. Prior to leaving on day 2 post surgery, the dog undergoes forcemat gaiting. Dogs are discharged to their owners on a standard course of rimadyl, a non-steroidal anti-inflammatory, for 5 days. For the next 12 days, dogs are given placebo or treatment oil every 12 hours. All dogs return two weeks after surgery for suture removal and have concurrent serum chemistry, serum collection for inflammatory biomarkers, and forcemat analysis performed. Eight weeks post operation, all dogs have radiographs taken to determine if healing is adequate and have blood collected for CBC, chemistry and markers of inflammation, as well as forcemat analysis. At weeks 0, 2 and 8 all owners complete a canine brief inventory of pain and Hudson scale scores for their dogs. Dogs are allocated to a treatment and a placebo group randomly in a double blinded fashion. All dogs undergo complete veterinary assessment for lameness, pain, and gait at the time of enrollment, week 2, and week 8 during routine visits.

### Statistical Analysis

The power of this study is assessed based on relative peak vertical force changes of approximately 5% difference across the groups at either 2 and/or 8 weeks post operation with a standard deviation of 8%, a standard alpha value of 0.05, and a beta value of 0.8. This reveals a need for at least 21 dogs in each group. All numerical and quantitative values are evaluated using a mixed model analysis of variance. Analyses take fixed effects into account, including weight, age, gender, neuter status and dog as a random effect with differences over time and treatment, as evaluated using Tukey's post hoc analysis. Significance is set at p<0.05 for all parameters.

### Example 18: Canine Pharmacokinetics

The oral pharmacokinetics of cannabidiol using industrial hemp have recently been established based on dosing at 2 mg/kg. The average 20 mg total dose resulted in a peak concentration of approximately 100 ug/kg. This peak concentration, a relative absorption rate of approximately 367 ug/L, and an average retention time of 6 hrs, suggest that total absorption is less than 20%. The absorption could potentially be improved using another matrix. However, lipid microspheres have not performed better than oil emulsions.

Prior work has suggested that lecithin may enhance the absorption of lipid emulsions in vivo and in situ. Therefore, the addition of sunflower lecithin to the base oil may provide a more effective medium for oral delivery.

In addition, biopolymer NF-971P, which is utilized in tablets and suspensions to lengthen delivery time of compounds, provides another option for oral delivery. NF-971F may require that the product be formulated into an aqueous suspension rather than an oil base.

All dogs have completed blood counts, serum chemistry and urinalysis performed at the end of the two week phases to ensure safety of the products.

### Study Design

### Phase 1

Dogs are dosed at 2 mg/kg at feeding using hemp extract in grapeseed oil. Following dosing, a 7 point pharmacokinetic analysis is performed. The dogs are dosed twice daily for two weeks. Blood samples are taken at the end of weeks 1 and 2 to examine the steady state levels in the bloodstream. Dogs are then washed out for 4 weeks.

### Phase 2

Dogs are dosed at 2 mg/kg with cannabidiol using a blend of the current oil with up to 40% sunflower lecithin. Following dosing, a 7 point, 24 hr pharmacokinetic study is performed. The dogs are then dosed twice daily for two weeks. Serum is collected at the end of weeks 1 and 2 to examine steady state levels. The dogs undergo a 4 week washout before the next phase of testing.

### Phase 3

The same suspension is used as in phase two with the addition of the biopolymer NF-971P at up to 2% weight/volume ratio. This creates a homogeneous suspension that is a liquid and is delivered in an oil based delivery system. This mixture allows for a longer digestion time and release of cannabinoids in the GI tract. This provides a more gradual peak and trough during the 24 hr pharmacokinetic testing. The emulsion is tested over a two week period with dosing twice a day (every 12 hrs). Serum is collected at the end of weeks 1 and 2. The dogs are then washed out for 4 weeks before the next phase of testing.

### Phase 4

Dogs undergo a 50 mg/ml transdermal application of 0.5 cc oil. Oil is administered intra-aurally every 12 hrs for 2 weeks. The dogs are housed separately during the study to prevent any oral exposure from grooming each other. A 7 point, 24 hr pharmacokinetic analysis is performed following the initial treatment. Blood is drawn at the end of weeks 1 and 2 to examine the steady state concentration in the bloodstream.

### Example 19: In vitro Cancer Study

In vitro cancer studies were conducted by treating cancer cell lines with CBD oil to examine the effects on cancer cell viability and apoptosis. Combination treatment with CBD oil and doxorubicin was also tested.

### Viability

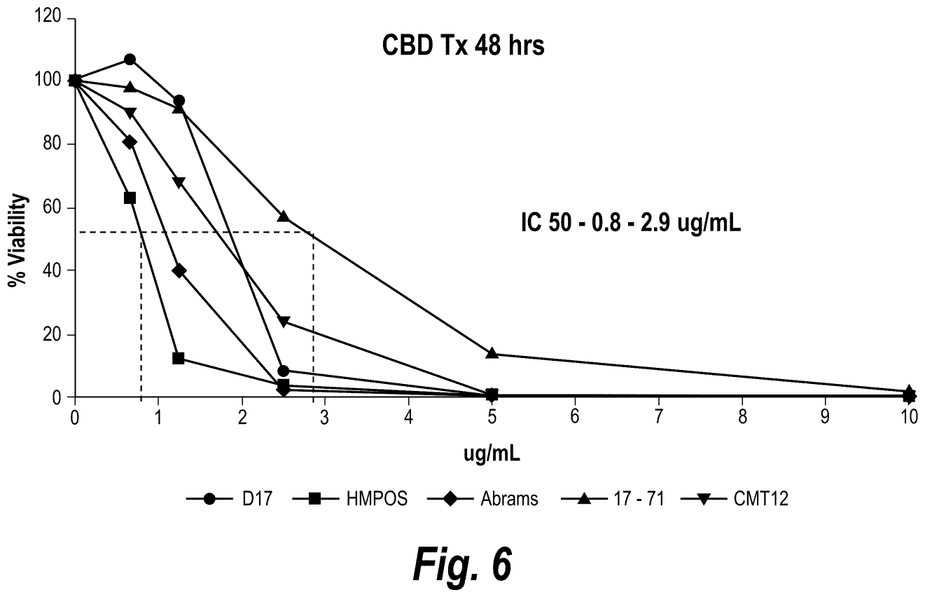

D17, HMPOS, Abrams, 17-71, and CMT12 cancer cell lines were treated with CBD oil. Cells were treated with a range of 0-10 μg/mL CBD oil over a 48 hour period. For all cells type, CBD oil caused a dose-dependent decrease in cell viability, with an IC₅₀ of 0.8-2.9 μg/mL, as shown in FIG. 6.

### Apoptosis

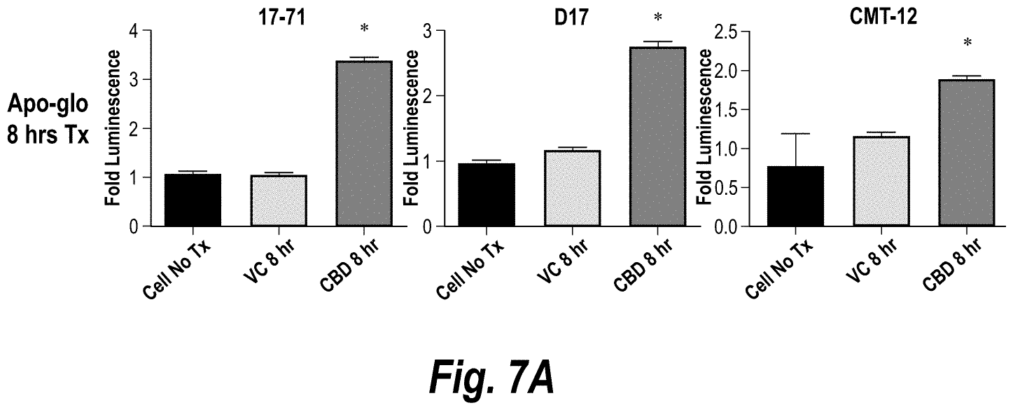

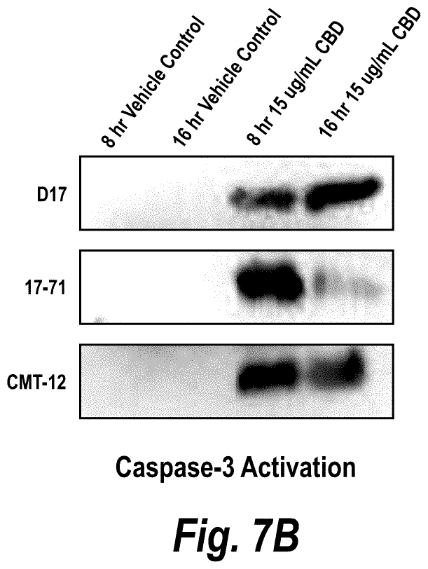

To measure apoptosis of cancer cells, D17, 17-71, and CMT12 cancer cell lines were treated with a vehicle control or CBD oil and incubated for 8 or 16 hours. At 8 hours, an Apo-glo assay was used to measure caspase activity as a marker of apoptosis. As shown in FIG. 7A, for all cell types, cells treated with CBD oil had a statistically significant increase in caspase activity compared to cells with no treatment or that were treated with a vehicle control. At 8 hours and 16 hours, western blotting was used to visualize caspase activation in the cancer cell lines, as shown in FIG. 7B. At both times points, caspase activity can be seen in cells treated with CBD oil, but not cells treated with vehicle control.

### Combination Index

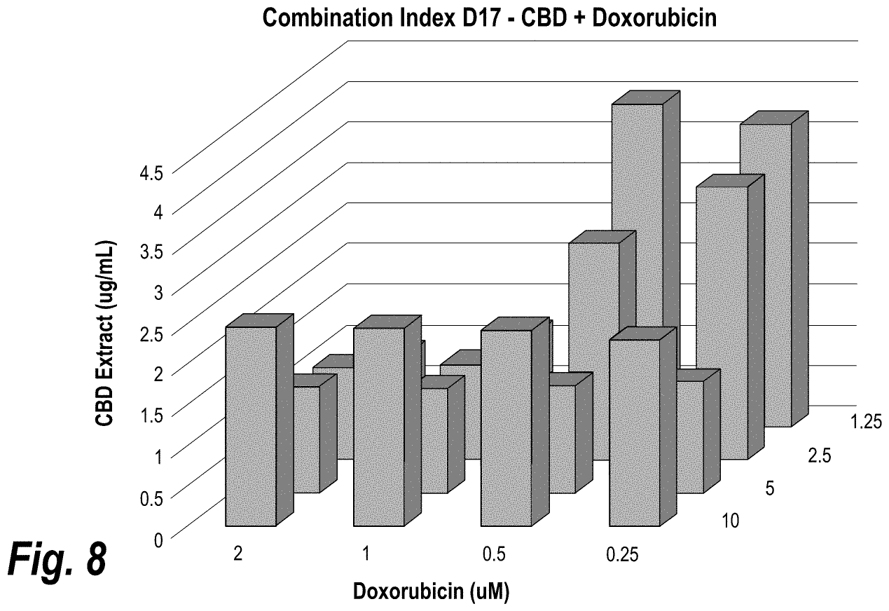

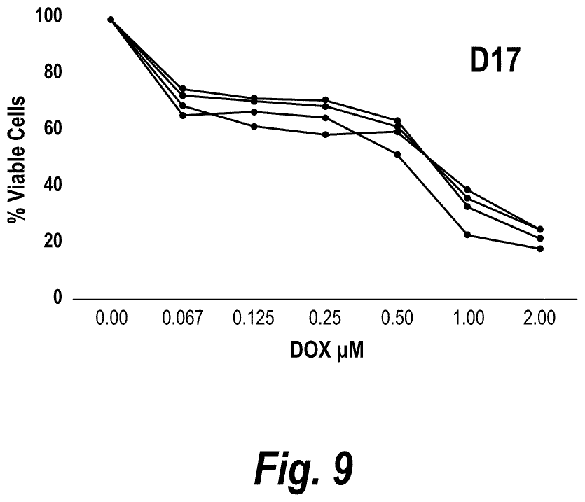

D17 cells were treated with a combination of CBD oil and doxorubicin at varying concentrations of each. FIG. 8 shows a synergistic relationship between CBD oil and doxorubicin when administered together.

### Example 20: CBD oil and Gabapentin Combination Treatment for Post-Operative Pain Relief in Canines

A study is being conducted to 1) evaluate safety and analgesic effect of CBD with gabapentin in the post-operative period following hemilaminectomy surgery by utilizing a validated pain scoring system and 2) directly compare the effectiveness of analgesia of CBD plus gabapentin (group 1) and gabapentin alone (group 2) in post-operative hemilaminectomy patients by comparing the time to first rescue and the number of rescue opioid injections needed in a 48 hour period.

### Study Design and Animals

Patients are selected from cases presenting for acute onset (less than 2-week duration of clinical signs), spinal pain, paraparesis or paraplegia, with a grade 1-4 Modified Frankel Spinal Cord Injury Score. Patients will have undergone hemilaminectomy between T10-L3 vertebral bodies and will have confirmed extruded disc material at time of surgery. Patients are excluded from the study if they are deep pain negative (Modified Frankel Spinal Cord Injury Score of 5) prior to or following surgery, have known food allergies, if routine preoperative blood work shows abnormalities, if patient temperament prevents evaluation of pain in the clinical setting, and if patients with corticosteroid use of greater than 1 week duration or at immunosuppressive dosages.

Sample size is calculated using a prior study which had a pooled standard deviation of 2.85 hours for time to opioid rescue. Using this standard deviation, power of 90%, α=0.05, and assuming an effect resulting in at least 2 hours of difference between groups, a priori sample size calculation is performed. With this calculation and consultation with Center for Interdisciplinary Statistical Education and Research (Pullman, Wash.), it was concluded that 19 dogs should be included in each group. To account for possible study drop-out, 25 dogs are assigned to both groups.

Patients are randomized into the following treatment groups utilizing a temporal randomized block design:
Treatment group 1: 10 mg/kg gabapentin and 8 mg/kg CBD oil given orally once every 8 hours
Treatment group 2: 10 mg/kg gabapentin and placebo oil given orally every 8 hours.

Once a treatment group is assigned, appropriate medications/placebos for patient weight is dispensed by the neurology specialty intern, who is unblinded.

### Materials

Liquid formulation gabapentin (50 mg/ml) (Amneal pharmaceuticals; Bridgewater, N.J.) is used for the study and dosed based on patient weight. Gabapentin is dosed orally at 10 mg/kg every 8 hours.

CBD oil is dosed orally at 8 mg/kg once every 8 hours. This dose was selected as the higher of the previously evaluated doses of 2 and 8 mg/kg that were demonstrated to be safe in dogs. Placebo CBD oil consists of olive oil with 10 parts per thousand of anise oil and 5 parts per thousand of peppermint oil.

When a patient is assigned to a treatment group, CBD oil or placebo oil is placed into a glass amber medicine vial and labeled based on patient information and weight. A commercial analytical lab (Integrity Labs, WA) analyzes cannabinoid concentration of CBD oils to evaluate the potential variation in concentration.

### Methods

All dogs undergo a standardized anesthesia protocol consisting of premedication with methadone (0.2 mg/kg) and dexmedetomidine (5 mcg/kg) IM or IV, followed by induction with propofol to effect and maintenance with isoflurane gas and fentanyl constant rate infusion (CRI). Fentanyl CRI (2-5 mcg/kg/min) is started at induction and discontinued when muscle layers are being closed. Intraoperative breakthrough pain is addressed with additional fentanyl boluses (2 mcg/kg). At time of fentanyl discontinuation, all patients receive an intramuscular dose of methadone (0.2 mg/kg). This dose of methadone is given to cover the immediate post-operative pain prior to when the patient can be assessed for pain or receive oral medications. This protocol of intraoperative fentanyl discontinuation, and methadone administration is similar to other studies evaluating post-operative hemilaminectomy pain in dogs.

All patients recover in the intensive care unit with routine monitoring and treatments. Patients begin oral treatments (as assigned by treatment group) after extubation when clinician observes intact gag reflex, alert and responsive mentation, the ability to maintain sternal recumbency without assistance.

A trained blinded observer records pain scores and any adverse side effects every 2 hours for the first 24 hours following surgery and every 4 hours for the following 24 hours. Pain scores are recorded using the previously validated Modified Glasgow Composite Scale, and scores out of 20 are recorded. Patients with a pain score greater than 5/20 at any point are rescued with methadone (0.2 mg/kg IV). The intervention of 5/20 has been previously validated and used in similar post-operative studies. The time to first rescue dose and the number of rescue doses over the 48 hour period is recorded.

Other observations that are recorded include: patient appetite and time at which patient begins eating regularly, patient neurologic grade assessed daily, any additional post-operative medications (such as maropitant or trazodone) and any adverse side-effects.

After study completion at 48 hours post-operative, a serum chemistry panel is repeated to re-evaluate liver values. Patients are continued on analgesic medications as dictated by their primary clinician. Patients are discharged from the hospital based on the discretion of the primary care clinician.

### Statistical Analysis

Commercially available statistical software is used to analyze the data. Differences between groups in age, sex, and neurologic grade is assessed using a Pearson Chi-Squared test. Time to first rescue opioid analgesia is compared via a Kaplan-Meier survival curve with a Breslow pairwise comparison. Differences in the total number of rescue opioids doses and pain score at presentation is assessed using a standard one-way ANOVA, provided the data is normally distributed. If the data is not normally distributed a Kruskal-Wallis one-way ANOVA is used. If a significant difference of means is observed between groups, a post-hoc Least Significant Difference is performed.

The safety of CBD alone and in combination with gabapentin is assessed. This is done by comparing the incidence of adverse effects between groups with a one-way ANOVA. If there is significant difference of means observed in the ANOVA, a post-hoc Tukey's multiple comparison test is performed. Statistical significance is considered at p<0.05.

### Example 21: Bioavailability and Metabolism of Cannabinoids

This study compares the absorption rate of three CBD formulations to the absorption rate of a chew based formulation by examining 24 hour pharmacokinetics and 1 and 2 week steady state levels.

### Study Design

### Phase 1

Six dogs were dosed at 2 mg/kg of CBD using sesame oil with 25% replacement of purified medium chain triglycerides (Oil A). Dogs were fed a half can of Purina Proplan at the time of dosing and were allowed to consume their daily meals after the 1 hr time point. A 7 point pharmacokinetics analysis was performed on the dogs. The dogs were then dosed twice daily (approximately 11 hrs apart) for two weeks. Blood samples were taken at the end of week 1 and week 2 to examine the steady state levels in the bloodstream. Dogs were then washed out for 3 weeks before phase 2.

### Phase 2

Six dogs were dosed at 2 mg/kg of CBD using sesame oil with 25% replacement of purified sunflower lecithin (Oil B). Dogs were fed a half can of Purina Proplan at the time of dosing and were allowed to consume their daily meals after the 1 hr time point. A 7 point pharmacokinetics analysis was performed on the dogs. The dogs were then dosed twice daily (approximately 11 hrs apart) for two weeks. Blood samples were taken at the end of week 1 and week 2 to examine the steady state levels in the bloodstream. Dogs were then washed out for 3 weeks before phase 3.

### Phase 3

Six dogs were dosed at 2 mg/kg of CBD using 10 mg chewable dosage forms. Dogs were fed a half can of Purina Proplan at the time of dosing and were allowed to consume their daily meals after the 1 hr time point. A 7 point pharmacokinetics analysis was performed on the dogs. The dogs were then dosed twice daily (approximately 11 hrs apart) for two weeks. Blood samples were taken at the end of week 1 and week 2 to examine the steady state levels in the bloodstream.

### Results

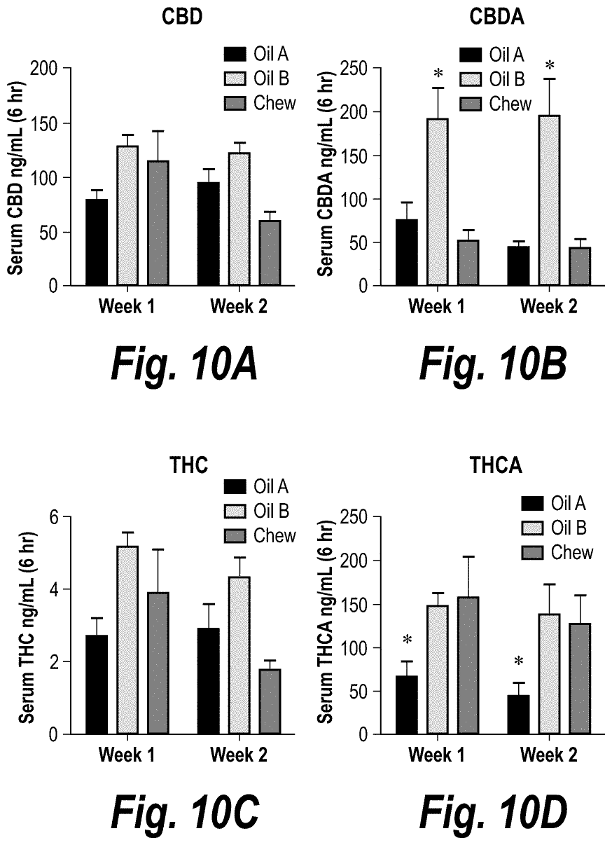

Table 17 shows data from the 24 hour pharmacokinetic analyses of Oil A, Oil B, and the chewables. Data is presented for the maximal concentrations observed in the blood (CMax), the time to reach maximal concentrations (TMax), the half life of the cannabinoid in the serum (T ½), the area under the curve over 24 hours (AUC-t), total area under the curve for the dose (AUC . . . ), and median retention time (MRT). In addition, a mean predicted serum concentration was calculated based on 5 half lives of each cannabinoid. A Kruskal-Wallis non-parametric test showed that there were only a few statistically significant differences (p value less than 0.05 with post hoc testing) across the three treatments for any of the parameters examined. This suggests that the 24 hour pharmacokinetics were similar for the three formulations. However, as shown in FIGS. 10A-10D, there were differences in the serum concentrations of CBD, CBDA, THC, and THCA concentrations across the week 1 and week 2 time points. A two-way analysis of variance over both time and treatment shows a treatment effect at both the week 1 and week 2 times points. The serum CBDA concentrations at 6 hours post dosing were higher for the Oil B group than the Oil A and chewables groups at both week 1 and 2. The THCA concentrations for the Oil B and chewables groups were higher than the Oil A group.

### Conclusion

These results suggest that all three of the CBD formulations are absorbed and can achieve physiological concentrations of CBD and CBDA based on current information (assuming 100 ng cannabinoid in serum is sufficient). Further, when using lecithin as 25% of the base, there is an apparent improvement in CBDA and THCA absorption and/or retention in the serum, suggesting that this formulation may require lower dosing than oils that do not contain lecithin to achieve similar cannabinoid concentrations.

TABLE 17
Pharmacokinetic Data for CBD formulations
Pharmacokinetic Data:

| | CMax | TMax | T 1/2 | AUC [symbol] | AUC [symbol] | MRT | Predict Ave | Week 1 Ave | Week 2 Ave |
|---|---|---|---|---|---|---|---|---|---|
| CBD (ng) | | | | | | | | | |
| Oil A | 145 ± 69 | 1.5 + 0.5 | 4.1 + 0.7 | 635 + 399 | 656 + 414 | 5.2 + 1.4 | 53 + 33 | 79 + 22 | 94 + 32 |
| Oil B | 124 + 62 | 2.0 + 1.1 | 4.4 + 1.4 | 683 + 146 | 707 + 144 | 6.5 + 2.1 | 63 + 17 | 228 + 24 | 122 + 25 |
| Chewable | 226 + 89 | 2.5 + 2.2 | 3.8 + 0.3 | 826 + 74 | 845 + 74 | 5.3 + 1.4 | 70 + 15 | 225 + 70 | 59 + 21 |
| CBDA (ng) | | | | | | | | | |
| Oil A | 383 + 167 | 1 + 0 | 4.4 + 2.7 | 1018 + 308 | 1152 + 451 | 5.2 + 3.3 | 88 + 41 | 75 + 52 | 44 + 18 |
| Oil B | 386 + 213 | 1.2 + 0.4 | 4.2 + 1.0 | 1619 + 898 | 1748 + 855 | 6.8 + 2.3 | 136 + 66 | 192 + 89 | 195 + 105 |
| Chewable | 510 + 350 | 2.3 + 0.6 | 2.4 + 1.1 | 1407 + 585 | 1419 + 591 | 4.3 + 1.5 | 191 + 158 | 52 + 29 | 44 + 10 |
| THC (ng) | | | | | | | | | |
| Oil A | | | | Insuffic. | Data | | | 3 + 1 | 3 + 1 |
| Oil B | 5 + 3 | 1.7 + 0.5 | 4.0 + 3.9 | 22 + 9 | 27 + 9 | 6.3 + 5.7 | 3.0 + 0.5 | 5 + 1 | 4 + 1 |
| Chewable | | | | Insuffic. | Data | | | 4 + 3 | 2 + 1 |
| THCA (ng) | | | | | | | | | |
| Oil A | 35 + 14 | 1.7 + 1.2 | 6.5 + 5.1 | 171 + 57 | 209 + 89 | 9.8 + 7.6 | 18 + 6 | 10 + 3 | 7 + 2 |
| Oil B | 27 + 21 | 2.2 + 1.0 | 5.9 + 2.5 | 256 + 114 | 291 + 119 | 8.7 + 3.7 | 25 + 10 | 24 + 3 | 22 + 5 |
| Chewable | 45 + 25 | 3.3 + 1.0 | 3.9 + 0.6 | 212 + 60 | 223 + 71 | 6.6 + 1.7 | 25 + 5 | 24 + 7 | 20 + 5 |

[symbol]

indicates data missing or illegible when filed

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A pharmaceutical composition comprising hemp extract and a carrier, wherein the hemp extract comprises:
cannabidiol; and
cannabidiolic acid;
wherein the ratio of cannabidiol to cannabidiolic acid is about 0.6:1 to about 1:0.6, and
wherein the carrier comprises biopolymer NF-971P and/or lecithin.

2. The pharmaceutical composition of claim 1, wherein the hemp extract further comprises:
cannabigerolic acid;
Δ9-tetrahydrocannabinol; and
cannabichromene.

3. The pharmaceutical composition of claim 1, wherein the hemp extract further comprises four or more of:
α-pinene;
β-myrcene;
β-pinene;
δ-limonene;
linalool;
β-caryophyllene;
α-humulene;
nerolidol 2;
guaiol;
caryophyllene oxide; and
α-bisabolol.

4. The pharmaceutical composition of claim 2, wherein the concentration of Δ9-tetrahydrocannabinol is insufficient to produce a psychotropic effect.

5.-17. (canceled)

18. The pharmaceutical composition of claim 1, wherein the carrier is selected from the group consisting of linseed oil, olive oil, fish oil, salmon oil, coconut oil, catnip oil, sesame oil, MCT oil, and grapeseed oil.

19. (canceled)

20. (canceled)

21. The pharmaceutical composition of claim 1, wherein the composition comprises lecithin.

22.-55. (canceled)

56. A dosage form comprising the pharmaceutical composition of claim 1 and one or more pharmaceutically acceptable additives, flavoring agents, surfactants, and adjuvants.

57.-68. (canceled)

69. The dosage form of claim 56, wherein the composition is formulated as a chew for oral administration.

70. The dosage form of claim 69, where the chew is produced using cold extrusion.

71. The dosage form of claim 70, wherein the weight of the chew is about 0.5-10 g.

72.-129. (canceled)

130. A method of treating epilepsy in a veterinary subject in need thereof, comprising administering a therapeutically effective amount of a pharmaceutical composition comprising hemp extract and a carrier, wherein the hemp extract comprises:
cannabidiol; and
cannabidiolic acid;
wherein the ratio of cannabidiol to cannabidiolic acid is about 0.6:1 to about 1:0.6.

131. A method of treating cancer in a veterinary subject in need thereof, comprising administering a therapeutically effective amount of a pharmaceutical composition comprising hemp extract and a carrier, wherein the hemp extract comprises:
cannabidiol; and
cannabidiolic acid;
wherein the ratio of cannabidiol to cannabidiolic acid is about 0.6:1 to about 1:0.6.

132. A method of improving quality of life in a veterinary subject with cancer, comprising administering a therapeutically effective amount of a pharmaceutical composition comprising hemp extract and a carrier, wherein the hemp extract comprises:
cannabidiol; and
cannabidiolic acid;
wherein the ratio of cannabidiol to cannabidiolic acid is about 0.6:1 to about 1:0.6.

133. The method of claim 130, wherein the hemp extract further comprises:
cannabigerolic acid;
Δ9-tetrahydrocannabinol; and
cannabichromene.

134. (canceled)

135. The method of claim 133, wherein the concentration of Δ9-tetrahydrocannabinol is insufficient to produce a psychotropic effect.

136.-149. (canceled)

150. The method of claim 130, wherein the carrier is selected from the group consisting of linseed oil, olive oil, fish oil, salmon oil, coconut oil, catnip oil, sesame oil, MCT oil, and grapeseed oil.

151.-185. (canceled)

186. The method of claim 130, comprising administering a dosage form comprising:
(a) the pharmaceutical composition, wherein the pharmaceutical composition further comprises:
(i) cannabigerolic acid,
(ii) Δ9-tetrahydrocannabinol,
(iii) cannabichromene; and
(b) one or more pharmaceutically acceptable additives, flavoring agents, surfactants, and adjuvants.

187.-221. (canceled)

222. The method of claim 186, wherein the carrier is selected from the group consisting of linseed oil, olive oil, fish oil, salmon oil, coconut oil, catnip oil, sesame oil, MCT oil, and grapeseed oil.

223.-261. (canceled)

262. The method of claim 130, wherein the composition comprises lecithin.

263. The method of claim 222, wherein the composition comprises lecithin.
